SEQUENCE LISTING (10 sequences)

SEQ ID NO: 1 - DNA, 21 bp, synthetic construct
  misc_feature 1..21: note = Synthetic DNA
  source 1..21: mol_type = other DNA
cctcatgaagatcctcaccga

SEQ ID NO: 2 - DNA, 21 bp, synthetic construct
  misc_feature 1..21: note = Synthetic DNA
  source 1..21: mol_type = other DNA
ttgccaatggtgatgacctgg

SEQ ID NO: 3 - DNA, 21 bp, synthetic construct
  misc_feature 1..21: note = Synthetic DNA
  source 1..21: mol_type = other DNA
aggaatggacttgaaacaagg

SEQ ID NO: 4 - DNA, 20 bp, synthetic construct
  misc_feature 1..20: note = Synthetic DNA
  source 1..20: mol_type = other DNA
gcaaagcttgttgatcatgg

SEQ ID NO: 5 - DNA, 23 bp, synthetic construct
  misc_feature 1..23: note = Synthetic DNA
  source 1..23: mol_type = other DNA
tcacaaagagatgatggaggaac

SEQ ID NO: 6 - DNA, 20 bp, synthetic construct
  misc_feature 1..20: note = Synthetic DNA
  source 1..20: mol_type = other DNA
acatgcaggtgagttgtcag

SEQ ID NO: 7 - DNA, 20 bp, synthetic construct
  misc_feature 1..20: note = Synthetic DNA
  source 1..20: mol_type = other DNA
atctgcacttcgtgtgcaag

SEQ ID NO: 8 - DNA, 23 bp, synthetic construct
  misc_feature 1..23: note = Synthetic DNA
  source 1..23: mol_type = other DNA
gagtctgaggatttccttagctc

SEQ ID NO: 9 - DNA, 20 bp, synthetic construct
  misc_feature 1..20: note = Synthetic DNA
  source 1..20: mol_type = other DNA
gctgagcctaatcctctgcc

SEQ ID NO: 10 - DNA, 20 bp, synthetic construct
  misc_feature 1..20: note = Synthetic DNA
  source 1..20: mol_type = other DNA
actgctcacttccaagaccg

(19) United States
(12) Patent Application Publication
(10) Pub. No.: US 2024/0352427 A1
(43) Pub. Date: Oct. 24, 2024

(54) MASS PRODUCTION METHOD OF PLURIPOTENT STEM CELL STOCK

(71) Applicant: KANEKA CORPORATION, Osaka (JP)

(72) Inventors: Sho KAMBAYASHI, Kobe-shi (JP); Yoshikazu KAWAI, Kobe-shi (JP)

(73) Assignee: KANEKA CORPORATION, Osaka (JP)

(21) Appl. No.: 18/747,005

(22) Filed: Jun. 18, 2024

Related U.S. Application Data
(63) Continuation of application No. PCT/JP2022/046407, filed on Dec. 16, 2022.

(30) Foreign Application Priority Data
Dec. 20, 2021 (JP) 2021-206065
Oct. 7, 2022 (JP) 2022-162712

(51) Int. Cl. C12N 5/074

(52) U.S. Cl. CPC C12N 5/0696; C12N 2501/603; C12N 2501/727

(57) ABSTRACT

The purpose of the present invention is to produce a large amount of a high-quality pluripotent stem cell stock. A mass production method of a pluripotent stem cell stock that comprises thawing starting cells followed by adhesion culture to thereby stabilize the cell conditions, and then growing the cells to a cell count that enables suspension culture. Subsequently, the cells are suspension cultured while precisely controlling the culture environment to thereby grow a large number of high-quality cells. Then, a stock is prepared at a low temperature from the cells having been grown by the suspension culture.

### CROSS-REFERENCE TO RELATED APPLICATIONS

This application is a Continuation of PCT International Application No. PCT/JP2022/046407, filed on Dec. 16, 2022, which claims priority under 35 U.S.C. 119 (a) to Patent Application No. 2021-206065, filed in Japan on Dec. 20, 2021 and Patent Application No. 2022-162712, filed in Japan on Oct. 7, 2022, all of which are hereby expressly incorporated by reference into the present application.

### REFERENCE TO SEQUENCE LISTING SUBMITTED VIA EFS-WEB

The application contains a Sequence Listing which has been submitted electronically in .XML format and is hereby incorporated by reference in its entirety. Said. XML copy, created on Sep. 29, 2022, is named “B210745 Sequence.xml” and is 13,515 bytes in size. The sequence listing contained in this .XML file is part of the specification and is hereby incorporated by reference herein in its entirety.

### TECHNICAL FIELD

The present invention relates to a large-scale method for producing a high-quality pluripotent stem cell stock by suspension culture, and to a method for improving the quality of a pluripotent stem cell.

### BACKGROUND ART

Pluripotent stem cells such as ES cells (embryonic stem cells) and iPS cells (induced pluripotent stem cells) have the ability to grow indefinitely and the ability to differentiate into various somatic cells. The practical application of a therapy in which somatic cells induced to differentiate from pluripotent stem cells is transplanted has the potential to fundamentally revolutionize therapy for intractable diseases and lifestyle-related diseases. For example, technologies have already been developed for inducing the differentiation of pluripotent stem cells into various somatic cells such as nerve cells, myocardial cells, blood cells, and retinal cells in vitro. In addition, an attempt is being made to culture HLA-homo iPS cells and universal iPS cells in large numbers, and produce a clinical iPS cell stock as a raw material in common, in which the HLA-homo iPS cells do not cause immunologic rejection to many people, and the universal iPS cells are gene-edited so as to cause immunologic rejection to nobody, when the people undergo transplantation of the somatic cell induced to differentiate.

However, there remain some problems with production of such a clinical iPS cell stock. Among them, one of the large problems is the efficient mass culture of iPS cells. In one example, it is said that approximately 1×10⁸ to 1×10⁹ cells per patient are necessary for treatment of heart disease. However, from a handleability viewpoint, the current common technological level has its limits in the number of cells that can be handled at one time, and hence, cells only for approximately 5 to 10 patients can be produced from one vial of an iPS cell stock in one cycle of production. On this premise, producing therapeutic cells for 10000 patients requires using 1000 to 2000 vials of iPS cell stocks (performing 1000 to 2000 cycles of production is required). Methods for culturing pluripotent stem cells are roughly classified, incidentally, into adherent culture, in which cells are adhered to a flat base material and cultured, and suspension culture, in which cells are cultured by suspending them in a liquid medium. In order to culture, for example, 1×10⁹ cells by common adherent culture, a base material having a total adhesion area of 10⁴ cm² or more is required, which, if converted into the number of ordinary 10 cm dishes, corresponds to approximately 100 to 200 dishes. Handling such a number of dishes in a production environment of clinical cells requires a great deal of handwork, and thus, is unrealistic. Thus, as a method for culturing a large number of pluripotent stem cells such as iPS cells as a raw material in common for cells for use for regenerative medicine, it is extremely difficult to utilize adherent culture on the surface of a base material having a culture area on which the number of cells to be obtained depends.

Meanwhile, in suspension culture, since cells are cultured while suspended or floating in a liquid medium, the number of cells to be obtained depends on the volume of the medium. Accordingly, the scale-up of suspension culture does not require such a vast culture area, compared with adherent culture, and allows culture in a narrow area (cell-preparing environment). Because of this, suspension culture is realistic, and suitable for the mass production of cells. For example, Non-Patent Literature 1 discloses a method of suspension culture for pluripotent stem cells while stirring a liquid medium using a spinner flask as a cell culture vessel for suspension culture. Non-Patent Literature 2 discloses a method for improving cell growth by a suspension culture method using a medium perfusion mode. However, any of these methods is premised on performing culture using good-quality cells as a raw material.

In addition, culturing pluripotent stem cells involves apprehension about a decrease in quality due to the progression of repeated passaging. Hence, it is considered that cells are preferably passaged and amplified the smallest possible number of times (Non-Patent Literature 3).

Incidentally, in the production of a cell stock of clinical iPS cells, it is important to produce a large amount of stock, and further, it is also very important that the stock is composed of iPS cells having good quality as well as a stable quality. Patent Literature 1 discloses a method in which cells in a suitable state are verified by monitoring the form of the cell colony, and then, a stock of high-quality pluripotent stem cells is produced. As above-mentioned, however, this method utilizes an adherent culture method, which is limitative in the scale-up of a cell stock. In addition, the monitoring method described cannot be applied to a suspension culture method, thus making it practically difficult to achieve mass culture.

In this regard, among existing various iPS cell lines, including research cell lines, clinical cell lines in particular are often insufficient in qualities such as a survival rate or an adhesion rate under the current situation owing to a factor such as being established under limited production environments and conditions in order to satisfy clinical criteria. Hence, it is not easy to produce a sufficient amount of suitable clinical iPS cell stock. Thus, it is further difficult to produce, using these iPS cell lines as raw materials, a large amount of iPS cell stock for use for production of a therapeutic somatic cell.

### CITATION LIST

### Non-Patent Literature

Non-Patent Literature 1: Olmer R. et al., Tissue Engineering: Part C, Volume 18 (10): 772-784 (2012)
Non-Patent Literature 2: Kropp C. et al., Stem Cells Translational Medicine, 5:1289-1301 (2016)
Non-Patent Literature 3: Bai Q. et al., Stem Cells And Development, 24 (5): 653-662 (2015)

### Patent Literature

Patent Literature 1: JP 2020-120613 A

### SUMMARY OF INVENTION

### Technical Problem

As above-described, although a method for mass-culturing pluripotent stem cells using a suspension culture method has been disclosed, the method disclosed as above has been revealed, according to the study of the present inventors, to be unsuitable for a method for efficiently culturing and amplifying clinical pluripotent stem cells (raw material cells) poor in qualities such as a survival rate or an adhesion rate. Accordingly, a technology, in which a high-quality pluripotent stem cell stock can be produced from poor-quality clinical pluripotent stem cells (raw material cells) with a suspension culture method suitable for scale-up, has not been established yet. Early development of such a technology and a production process is anticipated.

### Solution to Problem

The present inventors have vigorously studied to solve the above-described problems, and consequently discovered that, surprisingly, a high-quality cell stock can be produced extremely efficiently by thawing a freeze-preserved raw material cell, then first performing an adherent culture step for one or more passage periods, preferably two or more passage periods, and then performing a suspension culture step. In addition, the present inventors have completed the present invention through the discovery of suitable conditions under which each of the above-described steps of adherent culture and suspension culture is performed, as well as that preparing, before freezing, a cell stock after production under a specific condition enables the cell stock to be scaled up and mass-produced while the high quality is appropriately maintained.

The present invention encompasses the following:
(1) A method for producing a pluripotent stem cell stock, comprising:
(a) a step of thawing a frozen cell (typically a plurality of cells) as a raw material for a cell stock to be produced;
(b) a step of adherent culture of the raw material cell thawed;
(c) a step of suspension culture of the cell subjected to the adherent culture;
(d) a step of aliquoting the cell subjected to the suspension culture into a vessel for storing the stock; and
(e) a step of freezing the cell aliquoted into the vessel.
(2) The method for producing according to (1), wherein the number of the raw material cells used in the step (b) is 1×10⁶ cells or less, and the number of the cells upon termination of the culture in the step (c) is 1×10⁸ cells or more.
(3) The method for producing according to (1) or (2), wherein the adhesion rate of the raw material cells is 70% or less.

More preferably, the method for producing according to (1) or (2), wherein the adhesion rate of the raw material cells upon seeding in the step (b) is 70% or less.
(4) The method for producing according to any one of (1) to (3), wherein, in the adherent culture in the step (b), the cells are seeded at a density of 3×10³ cells/cm² or more.
(5) The method for producing according to any one of (1) to (3), wherein the step (b) comprises a passage.
(6) The method for producing according to (4), wherein the passage is a passage into a vessel having a larger area.

More preferably, the method for producing according to any one of (1) to (3), wherein the adherent culture in the step (b) is a method in which the cells thawed are seeded and cultured at a density of 3×10³ cells/cm² or more, and then passaged in a vessel having a larger area to be further subjected to adherent culture.
(7) The method for producing according to any one of (1) to (6), wherein the suspension culture in the step (c) is performed by a mode of perfusing a medium.
(8) The method for producing according to (7), wherein the mode of perfusing a culture medium comprises increasing the amount of medium perfused from an arbitrary time point in accordance with the growth of the cells.
(9) The method for producing according to (7) or (8), comprising controlling, by the mode of perfusing a medium, the amount of medium perfused so as to maintain the pH of a culture solution between 6.5 and 9.0.

More preferably, the method for producing according to (7) or (8), comprising maintaining the lactic acid concentration of the culture solution at 12 mM or less by the mode of perfusing a medium.
(10) The method for producing according to any one of (1) to (9), wherein the suspension culture in the step (c) comprises altering the concentration of carbon dioxide gas supplied in the range of from 10 to 0%, in accordance with the progress of the culture.
(11) The method for producing according to any one of (1) to (10), wherein the suspension culture in the step (c) is suspension stirring culture.
(12) The method for producing according to (11), wherein the suspension stirring culture comprises decreasing a stirring rate in a culture period.

More preferably, the method for producing according to (11), wherein the stirring rate for the suspension stirring culture is decreased in accordance with an increase in the size of a cell aggregate.
(13) The method for producing according to any one of (1) to (12), wherein the amount of the culture solution in the suspension culture in the step (c) is 100 mL or more.
(14) The method for producing according to any one of (1) to (13), wherein a specific growth rate of the cell upon termination of the step (c) is 0.70 day⁻¹ or more.

More preferably, the method for producing according to any one of (1) to (13), wherein a specific growth rate of the cell for 24 hours immediately before a shift from the step (c) to the step (d) is 0.7 day⁻¹ or more.
(15) The method for producing according to any one of (1) to (14), wherein the step (c) comprises a step of unicellularizing the cell aggregate.
(16) The method for producing according to (15), wherein the unicellularization comprises an enzymatic treatment in the presence of a ROCK inhibitor.
(17) The method for producing according to any one of (1) to (16), wherein, in the step (d), at least one of the vessels and a cell suspension is maintained at 10° C. or less.

More preferably, the method for producing according to any one of (1) to (16), wherein the step (d) is performed in a state where the vessel of the cell suspension is retained on a low-temperature base material of 10° C. or less, or performed under a low-temperature environment of 10° C. or less.
(18) The method for producing according to any one of (1) to (17), wherein 100 or more of the pluripotent stem cell stock are produced.

More preferably, the method for producing according to any one of (1) to (17), wherein the number of vessels, into which vessels the cells are aliquoted in the step (d), for storing the stock is 100 or more.
(19) The method for producing according to any one of (1) to (18), wherein the aliquoting in the step (d) is performed using a multi-channel pipette.

More preferably, the method for producing according to any one of (1) to (18), wherein the aliquoting in the step (d) is performed simultaneously for multiple vessels using a multi-channel pipette.
(20) The method for producing according to any one of (1) to (19), wherein the cells adhering undergo an enzymatic treatment in the presence of a ROCK inhibitor upon a shift from the step (b) to the step (c).
(21) The method for producing according to any one of (1) to (20), wherein the medium used for the culture in the step (c) comprises a ROCK inhibitor.

More preferably, the method for producing according to any one of (1) to (20), wherein the liquid medium in the step (c) comprises a ROCK inhibitor.
(22) The method for producing according to (21), wherein the ROCK inhibitor is Y-27632.
(23) The method for producing according to any one of (1) to (22), wherein the medium used for the culture in the step (b) and the step (c) comprises at least one selected from the group consisting of L-ascorbic acid, insulin, transferrin, selenium, and sodium hydrogen carbonate.

More preferably, the method for producing according to any one of (1) to (22), wherein the liquid medium in the step (b) and the step (c) contains at least one selected from the group consisting of L-ascorbic acid, insulin, transferrin, selenium, and sodium hydrogen carbonate.
(24) The method for producing according to any one of (1) to (23), wherein the medium used for the culture in the step (b) and the step (c) comprises FGF2 and/or TGF-β1.

More preferably, the method for producing according to any one of (1) to (23), wherein the liquid medium in the step (b) and the step (c) comprises FGF2 and/or TGF-β1.
(25) The method for producing according to any one of (1) to (24), wherein a proportion of the cells positive for OCT4 is 90% or more, and a proportion of the cells positive for TRA-1-60 is 90% or more, in the pluripotent stem cells constituting the stock.
(26) A method for improving the quality of a pluripotent stem cell, comprising (f) a step of adherent culture of a freeze-preserved pluripotent stem cell after being thawed, and (g) a step of suspension culture of the cell subjected to the adherent culture.
(27) The method according to (26), wherein the suspension culture in the step (g) is performed using a mode of perfusing a medium, and comprises increasing the amount of medium perfused in accordance with the growth of the cells.

More preferably, the method according to (26), wherein the suspension culture in the step (g) is performed using a mode of perfusing a medium, and comprises increasing the amount of medium perfused from an arbitrary time point in accordance with the growth of the cells.
(28) The method for producing according to (27), comprising controlling the amount of medium perfused so as to maintain the pH of a culture solution between 6.5 and 9.0 by the mode of perfusing the medium.

More preferably, the method according to (27), comprising maintaining the lactic acid concentration of the culture solution at 12 mM or less by the mode of perfusing the medium.
(29) The method according to any one of (26) to (28), wherein the suspension culture in the step (g) comprises altering the concentration of carbon dioxide gas supplied in the range of from 10 to 0%, in accordance with the progress of the culture.
(30) The method according to any one of (26) to (29), wherein the quality is the survival rate of a cell population.
(31) The method according to any one of (26) to (30), wherein the quality is the adhesion rate to a culture base material.
(32) The method according to (31), wherein the adhesion rate of the pluripotent stem cells used for the adherent culture in the step (f) is 70% or less.
(33) A pluripotent stem cell stock comprising a cell, wherein the survival rate of the cells is 90% or more after thawing, and wherein, when the cells are used for adherent culture after the thawing, the number of the cells adhering at hour 24 of culture is 0.8 times or more to the number of the cells seeded.
(34) A pluripotent stem cell stock comprising a cell, wherein the survival rate of the cells (number of viable cells divided by total number of cells) is 90% or more after thawing, and wherein, when the cells are used for suspension culture after the thawing, an aggregate-forming rate at hour 24 of culture is 0.8 times or more to the number of the cells seeded. In this section, “survival rate” means the proportion of number of viable cells with respect to the whole number of cells collected.
(35) A pluripotent stem cell stock, wherein, in respect of the cell cycle of the cell comprised, a proportion of the cells in the G2/M phase is 1.5 times or more with respect to a proportion of the cells in the G0/G1 phase.

The present specification encompasses the contents described in the specifications and/or drawings of Japanese Patent Application Nos. 2021-206065 and 2022-162712, on which the priority of the present application is based.

### Advantageous Effects of Invention

The present invention makes it possible that poor-quality clinical pluripotent stem cells are grown efficiently, that a pluripotent stem cell stock having high quality and a stable quality is mass-produced, and that the quality of the poor-quality clinical pluripotent stem cells is improved.

### BRIEF DESCRIPTION OF DRAWINGS

FIG. 1 is a characteristic diagram showing the transition of the carbon dioxide gas concentration when suspension culture was performed in Production Example 3.

FIG. 2 is a diagram showing a medium used when pluripotent stem cells are differentiated into three germ layers by a suspension rotation culture in Evaluation Example 4.

FIG. 3 illustrates characteristic graphs showing the three-germ-layer differentiation potency of a stock produced in each of Examples 1, 2, and 3, in which the potency was measured in Evaluation Example 4. In the graphs, the black bar denotes the result of the undifferentiated cells yet to be induced to differentiate. The “Detection Limit or Less” means that the expression level of the marker in the undifferentiated cell was equal to or lower than the detection limit. The obliquely shaded bars denote the results of the cells induced to differentiate.

FIG. 4 is a characteristic graph showing a difference in the cell survival rate between the stock produced in Comparative Example 1 and the stocks produced in Examples 1, 2, and 3, in which the cell survival rate was measured in Evaluation Example 6. In the graph, the error bars denote standard errors, and the symbols * mean that the p values are less than 0.05.

FIG. 5 is a characteristic graph showing a difference in the adhesion rate between the stock produced in Comparative Example 1 and the stocks produced in Examples 1, 2, and 3, in which the adhesion rate was measured in Evaluation Example 7 when the cells were thawed and then seeded in the adherent culture.

FIG. 6 is a characteristic graph showing a difference in the survival rate between the stock preparing methods in Example 4 and Examples 5 respectively, in which the survival rate was measured in Evaluation Example 10. In the graph, the standby time until freezing is indicated in units of minutes.

FIG. 7 is a characteristic graph showing a difference in the aggregate-forming rate in the suspension culture between the stock produced in Comparative Example 1 and the stocks produced in Examples 1, 2, and 3, in which the aggregate-forming rate was measured in Evaluation Example 11.

FIG. 8 is characteristic graphs showing the results of the cell cycle analysis of the stock produced in Comparative Example 1 and the stock produced in Example 1, in which the cell cycle analysis was performed in Evaluation Example 12.

### DESCRIPTION OF EMBODIMENTS

### 1. Method for Producing Pluripotent Stem Cell Stock

### 1-1. Outline

In a preferable method for producing a pluripotent stem cell stock according to the present invention, a high-quality pluripotent stem cell stock is produced as follows: cells as a raw material are thawed, then seeded at a high density, and subjected to adherent culture; then, the cells are efficiently amplified in large numbers by suspension culture in which the carbon dioxide gas concentration and the lactic acid concentration are controlled; and then, the resulting cells are prepared in a storable state and form at low temperature. A method for producing a pluripotent stem cell stock according to the present invention can mass-produce high-quality pluripotent stem cell stocks having a high survival rate and adhesion rate after thawing.

### 1-2. Definitions of Terms

The following terms used herein are defined.

### <<Cell>>

The “pluripotent stem cell” that is subjected to the invention herein has multipotency (pluripotency) capable of differentiating into all types of cells that constitute the living body, and refers to a cell that can continue to grow indefinitely while maintaining pluripotency in in-vitro culture under appropriate conditions. More specifically, pluripotency means the ability to differentiate into cells of all kinds of germ layers that constitute an individual (three germ layers of ectoderm, mesoderm, and endoderm in vertebrates). Examples of such cells include embryonic stem cells (ES cells), embryonic germ cells (EG cells), germline stem cells (GS cells), and induced pluripotent stem cells (iPS cells). An “ES cell” refers to a pluripotent stem cell prepared from an early embryo. An “EG cells” refer to pluripotent stem cells prepared from a fetal primordial germ cell (Shamblott M. J. et al., 1998, Proc. Natl. Acad. Sci. USA., 95:13726-13731). A “GS cell” refers to a pluripotent stem cell prepared from a testicular cell (particularly a sperm stem cell) (Conrad S., 2008, Nature, 456:344-349). In addition, an “iPS cell” refers to a pluripotent stem cell obtained by reprogramming that renders a somatic cell undifferentiated by introducing genes encoding a small number of reprogramming factors into a differentiated somatic cell.

A pluripotent stem cell as used herein may be a cell derived from a multicellular organism. A pluripotent stem cell is preferably an animal-derived cell or a mammal-derived cell. Examples of the mammal include, for example, rodents such as mice, rats, hamsters, and guinea pigs, livestock or pet animals such as dogs, cats, rabbits, bovines, horses, sheep, and goats, and primates such as humans, rhesus monkeys, gorillas, and chimpanzees. For example, human-derived cells may be appropriately used.

The pluripotent stem cell used herein includes a naïve pluripotent stem cell and a primed pluripotent stem cell. A naïve pluripotent stem cell is defined as a cell in a near-pluripotent state found in the pre-implantation inner cell mass. A primed pluripotent stem cell is defined as a cell in a near-pluripotent found in the post-implantation epiblast. A primed pluripotent stem cell is characterized by, compared to naïve pluripotent stem cells, less contribution to ontogenesis, only one transcriptionally active X chromosome, and higher levels of transcriptionally repressive histone modifications. In addition, a primed pluripotent stem cell marker is the OTX2 gene, and naïve pluripotent stem cell marker genes are the REX1 gene and KLF family gene. Further, the shape of colonies formed by primed pluripotent stem cells is the flat shape, and the shape of colonies formed by naïve pluripotent stem cells is the dome shape. In particular, a primed pluripotent stem cell may be appropriately used as pluripotent stem cells herein.

A pluripotent stem cell as used herein is preferably a cell that can be freeze-preserved, and be further grown as well as be kept pluripotent after thawing. The culture conditions used for the growth after thawing are not particularly limited. In addition, existing culture conditions that can grow the cell as well as keep the cell pluripotent may be used in the invention described herein, if any.

The pluripotent stem cell used herein may be a commercially available cell, a distributed cell, or a newly prepared cell. Although not limited, a pluripotent stem cell is preferably an iPS cell or an ES cell when used in each invention described herein.

When an iPS cell used herein is a commercially available iPS cell or a research cell line, examples of the cell line that may be used include, but are not limited to, 253G1 strain, 253G4 strain, 201B6 strain, 201B7 strain, 409B2 strain, 454E2 strain, 606A1 strain, 610B1 strain, 648A1 strain, HiPS-RIKEN-1A strain, HiPS-RIKEN-2A strain, HiPS-RIKEN-12A strain, Nips-B2 strain, TkDN4-M strain, TkDA3-1 strain, TkDA3-2 strain, TkDA3-4 strain, TkDA3-5 strain, TkDA3-9 strain, TkDA3-20 strain, hiPSC 38-2 strain, MSC-iPSC1 strain, BJ-iPSC1 strain, RPChiPS771-2 strain, WTC-11 strain, 1231A3 strain, 1383D2 strain, 1383D6 strain, 1210B2 strain, 1201C1 strain, and 1205B2 strain, HiPS-NB1RGB strain.

In addition, when the iPS cell line used herein is a clinical cell line, examples of the clinical cell line that may be used include, but are not limited to, QHJI01s01 strain, QHJI01s04 strain, QHJI14s03 strain, QHJI14s04 strain, Ff-I14s03 strain, Ff-I14s04 strain, and YZWI strain.

In addition, when the iPS cell used herein is prepared, genes of reprogramming factors to be introduced into a cell are not limited to any combination. For example, a combination of the OCT3/4 gene, the KLF4 gene, the SOX2 gene, and the c-Myc gene (Yu J, et al. 2007, Science, 318:1917-20) and a combination of the OCT3/4 gene, the SOX2 gene, the LIN28 gene, and the Nanog gene (Takahashi K, et al. 2007, Cell, 131:861-72) can be used. A method for introducing these genes into cells is not particularly limited, and may be, for example, the introduction of a gene as a nucleic acid, such as gene introduction using a plasmid such as an episomal vector or introduction of synthetic RNA, or the introduction as a protein. In addition, an iPS cell produced by a method using a Sendai virus vector, an untranslated RNA such as microRNA, a low-molecular-weight compound, or the like may also be used. Further, to suppress immunologic rejection, a universal iPS cell obtained by editing or removing the HLA gene may be used.

When an ES cell used herein is a commercially available ES cell, examples of the cell line that may be used include, but are not limited to, KhES-1 strain, KhES-2 strain, KhES-3 strain, KhES-4 strain, KhES-5 strain, SEES1 strain, SEES2 strain, SEES3 strain, SEES-4 strain, SEES-5 strain, SEES-6 strain, SEES-7 strain, HUES8 strain, CyT49 strain, H1 strain, H9 strain, and HS-181 strain.

### <<Pluripotent Stem Cell Population>>

As used herein, the “pluripotent stem cell population” refers to an assembly of cells composed of one or more cells comprising at least one pluripotent stem cell. A pluripotent stem cell population may consist solely of pluripotent stem cells or comprise other cells. The form of a pluripotent stem cell population is not particularly limited, and includes, for example, a tissue, a tissue fragment, a cell pellet, a cell aggregate, a cell sheet, a solution of suspended cells, a cell suspension, and a frozen product thereof. A pluripotent stem cell population used herein may comprise a plurality of pluripotent stem cell populations of a smaller size. All the small pluripotent stem cell populations contained in the pluripotent stem cell population may not be of the same morphology. In addition, a pluripotent stem cell population used herein may comprise cells in a single cell state. Preferably, the pluripotent stem cell population comprises a cell aggregate.

### <<Cell Aggregate>>

As used herein, the “cell aggregate” refers to an aggregated cell population formed by cell aggregation in suspension culture and is also called a spheroid. A cell aggregate is usually roughly spherical. A cell constituting a cell aggregate is not particularly limited as long as they comprise one or more types of pluripotent stem cells described above. For example, a cell aggregate composed of pluripotent stem cells such as human pluripotent stem cells or human embryonic stem cells comprises cells expressing pluripotent stem cell markers and/or positive for pluripotent stem cell markers.

A pluripotent stem cell marker is a gene that is specifically or excessively expressed in pluripotent stem cells. Examples thereof include Alkaline Phosphatase, Nanog, OCT4, SOX2, TRA-1-60, c-Myc, KLF4, LIN28, SSEA-4, SSEA-1, and combinations thereof.

A pluripotent stem cell marker can be detected by any detection method known in the art. For example, a method for detecting a cell marker includes, but are not limited to, flow cytometry and the various measurement methods below-described in relation to a three germ layer marker. For example, when flow cytometry is used as the detection method and a fluorescence-labeled antibody is used as the detection reagent, cells detected to have stronger fluorescence than the negative control (isotype control) may be regarded as a cell “positive” for the marker. The proportion of cells positive for the detection reagent (e.g., a fluorescence-labeled antibody analyzed by flow cytometry) is often referred to as the “positive rate” herein. In addition, when a labeled antibody is used as the detection reagent, any antibody known in the art may be used. Examples of the fluorescence-labeled antibody include, but are not limited to, an antibody labeled with fluorescein isothiocyanate (FITC), phycoerythrin (PE), allophycocyanin (APC), or the like.

The proportion of pluripotent stem cells that constitute a cell aggregate can be determined, for example, by the positive rate for the pluripotent stem cell marker. The positive rate for the pluripotent stem cell marker in cells constituting a cell aggregate may be preferably 80% or more, more preferably 90% or more, for example, 91% or more, for example, 92% or more, for example, 93% or more, for example, 94% or more, for example, 95% or more, for example, 96% or more, for example, 97% or more, for example, 98% or more, for example, 99% or more, for example, 100%. A cell aggregate in which the proportion of cells expressing a pluripotent stem cell marker and/or the proportion of cells positive for the pluripotent stem cell marker is/are within the above-described range is a highly undifferentiated and more homogeneous cell population.

The proportion of cells expressing a pluripotent stem cell marker in cells constituting a cell aggregate may be preferably 80% or more, more preferably 90% or more, for example, 91% or more, for example, 92% or more, for example, 93% or more, for example, 94% or more, for example, 95% or more, for example, 96% or more, for example, 97% or more, for example, 98% or more, for example, 99% or more, for example, 100%.

The proportion of pluripotent stem cells may be determined by detecting the expression of one or more, two or more, or three or more pluripotent stem cell markers. In this case, the number of pluripotent stem cell marker within the above-described numerical range is not particularly limited. It may be, for example, one or more, two or more, three or more, or all of the pluripotent stem cell markers detected.

### <<Adherent Culture>>

“Adherent culture” is a cell culture method, and refers to culturing cells by attaching the cells to an external matrix or the like present on the surface of a culture vessel or the like. In a typical adherent culture, cells are grown in a single layer. As an external matrix, for example, laminin, vitronectin, gelatin, collagen, E-cadherin chimeric antibody, and any combination thereof may be used but is not particularly limited thereto. Cells subjected to adherent culture are grown to form a cell colony dense with the cells. In general, the pluripotent stem cells described above can be cultured not only by adherent culture but also by suspension culture.

### <<Suspension Culture>>

“Suspension culture” is one of the cell culture methods and refers to culturing cells in a liquid medium in a suspension state. As used herein, the “suspension state” refers to a state in which cells are not fixed by attachment or the like to an external matrix on the surface of a culture vessel (e.g., the inner surface such as the wall surface, the bottom surface, the lower surface of the cover, or the surface of the structure inside the culture vessel (e.g., a stirring blade)). The “suspension culture method” is a method for culturing cells by suspension culture. The cell in this method exists as aggregated cell masses in the culture solution. A method for suspending cells is not particularly limited, and includes stirring, rotation, and shaking. In addition, for example, a culture method in which cells are attached to a microcarrier, and cultured in a suspended state in a culture solution is herein regarded as suspension culture because the whole cell mass containing a microcarrier is suspended without being fixed to a culture vessel, although the cells are adhering to a microcarrier. In general, the above-described cells can be cultured not only by suspension culture but also by adherent culture.

### <<Medium and Mode of Medium Change>>

As used herein, the “medium” refers to a liquid or solid substance prepared for culturing cells. In principle, a medium contains at least the minimum necessary amount of components essential for growth and/or maintenance of a cell. Unless otherwise specified, the medium herein corresponds to a liquid medium for animal cells used for culturing animal-derived cells. As used herein, a liquid medium is often abbreviated simply as “medium.”

As used herein, the “basal medium” refers to a medium that is the basis for the media for various animal cell culture. Culture may be possible with a basal medium alone, but by adding various culture additives, it is also possible to prepare a medium according to the purpose, for example, a medium specific to various cells. A basal medium used herein includes, but is not particularly limited to, BME medium, BGJb medium, CMRL1066 medium, Glasgow MEM, Improved MEM Zinc Option medium, Iscove's Modified Dulbecco's Medium (IMDM), Medium 199, Eagle MEM, αMEM, Dulbecco's Modified Eagle's Medium (DMEM), Ham's F10 medium, Ham's F12 medium, RPMI 1640 medium, Fischer's medium, and mixed medium thereof (e.g., DMEM/F12 medium (Dulbecco's Modified Eagle's Medium/Nutrient Mixture F-12 Ham)). As DMEM/F12 medium, it is particularly preferable to use a medium obtained by mixing DMEM medium and Ham's F12 medium at a weight ratio in a range of 60/40 or more and 40/60 or less, for example, 58/42, 55/45, 52/48, 50/50, 48/52, 45/55, or 42/58. Other media such as those used for culturing human iPS cells and human ES cells may also be appropriately used.

Examples of preferable media that can be used in the present invention include media that do not contain serum, that is, serum-free media.

As used herein, a “culture additive” is a substance other than serum and gaseous components added for culturing to a medium. Specific examples of a culture additive include, but are not limited to, L-ascorbic acid, insulin, transferrin, selenium, sodium hydrogen carbonate, growth factors, fatty acids or lipids, amino acids (e.g., non-essential amino acids), vitamins, cytokines, antioxidants, 2-mercaptoethanol, pyruvic acid, buffers, inorganic salts, antibiotics, and any combination thereof. Insulin, transferrin, and cytokines may be naturally occurring proteins isolated from tissues or serum of animals (e.g., humans, mice, rats, bovines, horses, and goats) or may be genetically engineered recombinant proteins. Further, as a growth factor, for example, basic fibroblast growth factor-2 (FGF2), transforming growth factor-β1 (TGF-β1), Activin A, IGF-1, MCP-1, IL-6, PAI, PEDF, IGFBP-2, LIF, IGFBP-7, and any combination thereof may be used, but is not limited thereto. An antibiotic, for example, penicillin, streptomycin, amphotericin B, or any combination thereof may be used, but is not limited thereto. A growth factor such as FGF2 and/or TGF-β1 may be preferably used as a culture additive for the medium used in the present invention.

In addition, preferably, the medium contains a ROCK inhibitor. Examples of ROCK inhibitor include Y-27632. By containing a ROCK inhibitor in the medium, cell death under not-adhered state of pluripotent stem cells to a substrate or other cells and/or high shear stress can be remarkably suppressed. However, in adherent culture, continuous addition of Y-27632 induces cellular atypia, and thus, it is preferable that, after the cell has formed a colony, the culture medium not containing Y-27632 is used.

In addition, the culture medium preferably contains a protein kinase Cβ (PKCβ) inhibitor and/or a WNT inhibitor to maintain or enhance the undifferentiated property of a pluripotent stem cell. Examples of the PKCβ inhibitor include LY333531, Go6983, and GF109203X. Examples of the WNT inhibitor include IWR-1-endo, XAV939, WNT-C59, IWP-2, IWP-3, and the like. Adding such an inhibitor can suppress the spontaneous differentiation of a pluripotent stem cell and the deterioration of the quality, and stabilize the cells in the culture.

Further, the medium preferably has a composition that does not contain LIF when a primed pluripotent stem cell is to be cultured. Further, the medium composition preferably does not contain either one or both of a GSK3 inhibitor and/or an MEK/ERK inhibitor when a primed pluripotent stem cell is to be cultured. It is possible to culture a primed pluripotent stem cell while maintaining their undifferentiated state without making the primed pluripotent stem cells naïve in a medium that does not contain any of LIF, a GSK3 inhibitor, and an MEK/ERK inhibitor.

The medium used in the present invention may contain one or more culture additives described above. In general, examples of a medium to be supplemented with the culture additives include, but are not limited to, the basal medium.

A culture additive can be added to the medium directly or in the form of a solution, a derivative, a salt, a mixed reagent, or the like. For example, L-ascorbic acid may be added to the medium in the form of a derivative such as magnesium ascorbate 2-phosphate. Selenium may be added to the medium in the form of a selenite salt (e.g., sodium selenite). In addition, insulin, transferrin, and selenium may also be added to the medium in the form of an insulin-transferrin-selenium (ITS) reagent. A commercially available medium supplemented with these culture additives, for example, a commercially available medium supplemented with at least one selected from L-ascorbic acid, insulin, transferrin, selenium, or sodium hydrogen carbonate may also be used. Examples of commercially available media supplemented with insulin and transferrin include CHO-S-SFM II (Life Technologies Japan Ltd.), Hybridoma-SFM (Life Technologies Japan Ltd.), eRDF Dry Powdered Media (Life Technologies Japan Ltd.), UltraCULTURE™ (BioWhittaker), UltraDOMA™ (BioWhittaker), UltraCHO™ (BioWhittaker), UltraMDCK™ (BioWhittaker), STEMPRO (registered trademark) hESC SFM (Life Technologies Japan Ltd.), Essential 8™ (Life Technologies Japan Ltd.), StemFit (registered trademark) AK02N (AJINOMOTO CO., INC.), StemFit (registered trademark) AK03N (AJINOMOTO CO., INC.), mTeSR1 (Veritas), and TeSR2 (Veritas).

As used herein, the “mode of the medium change” refers to a method for supplying a medium, as a nutrient source, to cells for survival and growth of a cell and a method for removing a medium in which nutrients have been consumed by cells and metabolites have accumulated. The mode of the medium change includes, for example, and is not particularly limited to, the batch mode and the perfusion mode. The batch mode refers to replacing an arbitrary amount (e.g., whole amount, half amount) of the medium in the culture system (herein often referred to as “culture solution”) with a new medium at arbitrary culture intervals. The perfusion mode refers to continuous medium change by removing and separately supplying the medium in the culture system continuously. The amount of medium removed and supplied per unit time is referred to as the amount of medium perfused. Medium perfusion may be performed continuously or intermittently at multiple times. In suspension culture, a medium is preferably changed by a perfusion mode.

### <<Gas Supply>>

As used herein, “gas supply” refers to supplying oxygen and carbon dioxide necessary for survival and/or growth or the like of a cell to the culture solution by gas aeration through the culture solution in which a cell is cultured. A component of gas used for gas supply include oxygen, nitrogen, carbon dioxide, and other gas components present in the atmosphere. Regarding the proportions of components in the gas supplied, the lower limit of oxygen is preferably 1%, 2%, 3%, 4%, 5%, 10%, or 20%, and the upper limit thereof is preferably 100%, 90%, 80%, 70%, 60%, 50%, 40%, 30%, or 20%. The lower limit of a proportion of carbon dioxide is preferably 5%, 4%, 3%, 2%, 1%, or 0%, and the upper limit thereof is preferably 20%, 10%, 9%, 8%, 7%, 6%, or 5%. As the proportion of each of oxygen and carbon dioxide, any proportion may be selected, independent of each other. A method for adjusting the proportion of each of oxygen and carbon dioxide is not particularly limited. For example, by adding nitrogen as a component other than oxygen and carbon dioxide, the oxygen concentration and the carbon dioxide concentration in the gas may be adjusted. In addition, a method for preparing the supply gas is not particularly limited. For example, the supply gas may be prepared by mixing oxygen, carbon dioxide and nitrogen, each purified, or by mixing air with oxygen, carbon dioxide, or nitrogen. For example, the proportion of oxygen:carbon dioxide:nitrogen in the gas supplied includes, but are not limited to, 20:5:75, 20:4:76, 20:3:77, 20:2:78, 20:1:79, 20:0:80, 5:5:90, 5:0:95, 40:5:55, and 50:0:50. The proportion may not be constant during culturing, and may be changed at any time. Examples of the method for gas supply include: a method in which a tube is connected to a bioreactor or the like, and gas is actively sent out for gas aeration; and a method in which an incubator is filled with gas having any composition, and the gas is supplied to a culture vessel in a naturally diffusive manner. The gas supplied to the cell culture solution is preferably sterile and is, but is not limited to, preferably supplied to the culture solution through a filter. As used herein, the “carbon dioxide” may be referred to as “carbon dioxide gas,” and the “carbon dioxide concentration in the gas supplied” may be referred to as “carbon dioxide gas concentration.” In addition, the “carbon dioxide concentration in the liquid medium” may be referred to as “dissolved carbon dioxide gas concentration.”

### <<Cell Stock>>

As used herein, a “cell stock” is any number of cells from a pluripotent stem cell population of the same cell line and of the same origin, the cells being subdivided and stored. A vessel for the subdivision and storage is not particularly limited. For example, a vial, bag, or the like can be used. In addition, the cells in the cell stock are suspended in a storage solution. The composition of the storage solution is not particularly limited. For example, any culture medium supplemented with DMSO at a final concentration of 10% may be used. A commercially available storage solution may be used, or another storage solution may be used. Examples of a commercially available storage solution include STEM-CELLBANKER (registered trademark) GMP grade (Zenogen Pharma Co., Ltd.), CryoStor (registered trademark) CS10 (HemaCare Corporation), CP-5E (Kyokuto Pharmaceutical Industrial Co., Ltd.), a Ringer's solution, and a lactate Ringer's solution. The form of a storage solution having cells suspended therein is not particularly limited. The solution may be, for example, in the form of a liquid, viscous liquid, or gel. In a case where the cell stock is freeze-preserved, the stock may be frozen as a solid. The cells may be in a unicellular state, may be in a clumped state in which a plurality of cells adhere to each other, may be in a cell-aggregate state, and is preferably in mixture of a unicellular state and a clumped state, or in a unicellular state. In addition, the storage state of the cell stock is not particularly specified, and may be cold storage, or may be freezing storage. The freezing storage method is not particularly limited, and, for example, may be storage in a freezer at −80° C., may be storage in a gas phase on liquid nitrogen, or may be storage in the liquid phase of liquid nitrogen.

As used herein, a “cell stock” is referred to as a “pluripotent stem cell stock”, “stock”, or “frozen stock” in some cases. In addition, subdividing cells into a plurality of vessels is referred to as “filling” or “aliquoting” in some cases. Filling a vessel with cells suspended in a storage solution is referred to as “preparing a cell stock” in some cases.

### 1-3. Method for Producing Cell Stock

A method in the present aspect essentially comprises an adherent culture step of a raw material cell (a starting material cell or a source cell), a subsequent suspension culture step, and a further subsequently, cell stock preparing step. In addition, the method in this aspect may comprise a step of freezing. Each of the steps will be described below.

### 1-3-1. Adherent Culture Step

The “adherent culture step” is a step in which cells (e.g., rare cells) as a raw material are recovered from damage due to storage, and simultaneously grown to such a number of cells as can be seeded in suspension culture on a scale sufficient to produce a cell stock efficiently. Rare cells are raw material cells that are rare. For example, iPS cells, which are one of the raw material cells in the present invention, are “rare” cells because they are expensive and are usually provided in small amounts. However, “rare” cells are only an example of the raw material cells, and the raw material cells in the present invention are not necessarily “rare”. For adherent culture, an animal cell culture method known in the art may be used. For example, the adherent culture may allow cells to adhere to a culture base material such as a vessel or a carrier, and be simultaneously cultured.

### (Raw Material Cell)

A cell (raw material cell) used as a raw material (starting material) in this step is a cell that can be subjected to adherent culture, and also enables cell aggregation in the below-described suspension culture. As described in “1-2. Definitions of Terms” above for “pluripotent stem cell,” for example, an animal cell or a human cell is preferable. In addition, the kind of a cell that can be appropriately used is a pluripotent stem cell such as an iPS cell and an ES cell. Specifically, for example, the QHJI14 strain, which is an iPS cell line of an HLA-homo type the most frequent in the Japanese, can be used. The pluripotent stem cell used in this step may be a cell population (pluripotent stem cell population) consisting of a plurality of cells. When the pluripotent stem cell is a pluripotent stem cell population, the proportion (percentage) of cells expressing pluripotent stem cell markers (e.g., OCT4, SOX2, NANOG) and/or cells positive for pluripotent stem cell markers in the cell population is, for example, 90% or more, 91% or more, 92% or more, 93% or more, 94% or more, 95% or more, 96% or more, 97% or more, 98% or more, 99% or more, or 100%. The cells used in this step may be of one kind or a plurality of kinds. In addition, the cells may be a cell line of one specific kind, or may be a mixture of a plurality of kinds of cell lines.

In addition, raw material cells stored in a frozen state or the like can be used. In addition, it is possible in the present invention to use, in adherent culture as the first culture step, a raw material cell that is a viable cell the adhesion rate of which on a culture base material after seeding is originally low or decreased by some factor, owing to the nature of the cell line. The adhesion rate at the start of the culture may be, for example, 70% or less, 60% or less, 50% or less, 40% or less, or 30% or less. Further, the pluripotent stem cells used as a raw material is often rare by nature, the number of such cells usable as a raw material is often small. In the present invention, however, cells even in very small numbers can be handled as a raw material. The upper limit of the number of raw material cells is not particularly limited. Specifically, the number of raw material cells may be 1.5×10⁶ cells or less, 1.2×10⁶ cells or less, 1.0×10⁶ cells or less, 0.8×10⁶ cells or less, 0.6×10⁶ cells or less, 0.5×10⁶ cells or less, 0.4×10⁶ cells or less, 0.3×10⁶ cells or less, or 0.2×10⁶ cells or less. In addition, the lower limit of the number of raw material cells is not particularly limited. Raw material cells, even if in considerably small numbers, can be used in the method according to the present invention. The number may be, for example, 0.01×10⁶ cells or more, 0.05×10⁶ cells or more, 0.1×10⁶ cells or more, 0.125×10⁶ cells or more, 0.14×10⁶ cells or more, 0.15×10⁶ cells or more, 0.16×10⁶ cells or more, 0.175×10⁶ cells or more, or 0.2×10⁶ cells or more. In addition, in the present invention, a clinical cell line, whose cells are not only small in number but also of unstable quality, can be used as a raw material cell.

The above-described raw material cells are usually sold commercially, distributed, or stored in a frozen state. Accordingly, in the present invention, in a case where frozen cells are used as a raw material cell, the frozen cells as a raw material need to be thawed before use for an adherent culture step. The thawing condition in this case is not particularly limited. The cells are thawed preferably by heating rapidly. Thawing cells by heating rapidly refers to bringing the temperature of the cells to more than 0° C. within a predetermined period of time. Specifically, the temperature of the cells is brought to more than 0° C., for example, within 5 minutes, within 3 minutes, within 2 minutes, within 1.5 minutes, or within 1 minute. A method for heating rapidly is not particularly limited. Examples include a method in which a vessel filled with frozen cells is set, for example, in a water bath, ethanol bath, dry bath, or the like kept at a temperature of approximately 37° C. Alternatively, a cell-thawing device such as ThawSTAR (BioLife Solutions Inc.) may be used.

### (Culture Vessel)

The culture vessel used for adherent culture is not particularly limited. A culture vessel on the inner surface of which no treatment was performed for suppressing protein adsorption is preferable. In addition, a culture vessel that can be coated with an external matrix is preferable. Examples of a vessel that may be used include a vessel coated with an external matrix, a vessel treated for cell adhesion by a method other than the external matrix coating, and a vessel composed of a material adherent to cells, such as plastic. For example, the shape of the culture vessel includes, but is not particularly limited to, dish-shaped, flask-shaped, well-shaped, and bag-shaped culture vessels. For example, a cell culture flask (TPP Techno Plastic Products AG) can be used as a culture vessel.

The capacity of a culture vessel to be used can be appropriately selected without particular limitation. It is preferable that the lower limit of the area of the bottom face of the portion for accommodating a culture medium in a planar view (that is, the area of the bottom), is 0.32 cm², 0.65 cm², 1.9 cm², 3.0 cm², 3.5 cm², 9.0 cm², or 9.6 cm², or 10.0 cm², 15.0 cm², 20.0 cm², 21.0 cm², 22.5 cm², 24.0 cm², or 25.0 cm², and that the upper limit is 1000 cm², 500 cm², 400 cm², 300 cm², 200 cm², 150 cm², 100 cm², 75 cm², 50 cm², or 25 cm².

The capacity of the culture vessel to be used may be appropriately selected and is not particularly limited. The lower limit of the volume capable of accommodating a medium and of culturing is preferably 0.5 mL, 1 mL, 2 mL, 4 mL, 10 mL, 20 mL, 30 mL, 50 mL, or 100 mL, and the upper limit thereof is preferably 1 L, 500 mL, 200 mL, or 150 mL.

### (External Matrix)

An external matrix to be used for adherent culture is that to which an iPS cell such as Laminin or Vitronectin can adhere. Examples of the external matrix as a commercially available product include iMatrix-511 (Matrixome, Inc.) and Vitronectin-N (Thermo Fisher Scientific Inc.). When raw material cells are seeded, an external matrix to be used is preferably Laminin such as iMatrix-511 (Matrixome, Inc.). Laminin has a strong adhesive force to an iPS cell. When a small number of unstable cells are seeded, Laminin enables more of the cells to adhere in a viable state.

Meanwhile, in a case where adherent culture is performed for two or more passage periods (passage is performed once or more), Vitronectin is preferably used as an external matrix in adherent culture immediately before shifting to a suspension culture. Vitronectin has a weaker adhesive force to iPS cells than Laminin, and thus, enables iPS cells to be detached from a culture base material with less stimulus, and shift to suspension culture with less damage. In addition, it is considered that, in a case where Vitronectin is used, the weaker adhesion to a culture base material provides stronger adhesion between the cells, and generates a state relatively similar to cell aggregation in suspension culture, thus allowing a smooth shift to suspension culture.

### (Medium)

A medium to be used in adherent culture is a medium described in “1-2. Definitions of Terms” above, and is not limited as long as the medium can grow and/or maintain pluripotent stem cells. It is particularly preferable to use a medium that does not contain leukemia inhibitory factor. In addition, a medium to be used in the present invention is preferably a liquid medium containing L-ascorbic acid, insulin, transferrin, selenium, and/or sodium hydrogen carbonate. In addition, the liquid medium preferably contains at least one growth factor, and the liquid medium more preferably contains FGF2 and/or TGF-β1 as the growth factor. For example, a serum-free DMEM/F12 medium containing L-ascorbic acid, insulin, transferrin, selenium, sodium hydrogen carbonate, FGF2, and TGF-β1 may also be appropriately used. In addition, the culture medium preferably contains a ROCK inhibitor from the time when cells are seeded to the time when the cells seeded adhere to a vessel, or to the time when the cells seeded form a colony. The concentration of the ROCK inhibitor is not particularly limited. The upper limit of the concentration may be, for example, 40 μM, 30 μM, or 20 μM, and the lower limit may be, for example, 2 μM, 2.5 μM, 3 μM, 5 μM, 7 μM, 8 μM, 9 μM, or 10 μM. The concentration of the ROCK inhibitor may be constant in a culture, or may change. In addition, the period of time when a medium containing a ROCK inhibitor is used is not particularly limited. For example, the lower limit of the period of time when a culture medium containing a ROCK inhibitor is used may be 12 hours, 16 hours, 20 hours, or 24 hours, and the upper limit may be 28 hours, 32 hours, 36 hours, 40 hours, 44 hours, 48 hours, 52 hours, or 56 hours after cells are seeded. Adherent culture in the medium containing a ROCK inhibitor can suppress cell death during the shift to the subsequent suspension culture step. In addition, the ROCK inhibitor may be added for only a part of the period of time of the adherent culture. For example, the medium containing a ROCK inhibitor may be used only for 1 day, 2 days, or 3 days or more immediately before the shift to suspension culture.

In a case where a PKCβ inhibitor is used, the lower limit of the concentration of the PKCβ inhibitor is not particularly limited, and can be selected in a range that can suppress deviation from the undifferentiated state.

For example, the final concentration of the PKCβ inhibitor in a liquid medium may be 25 nM or more, 30 nM or more, 50 nM or more, 80 nM or more, 100 nM or more, 150 nM or more, 200 nM or more, 500 nM or more, or 700 nM or more. In addition, the final concentration may be, for example, 900 nM or more, 1 μM or more, or 1.1 μM or more. The upper limit of the concentration of the PKCβ inhibitor is not particularly limited, and may be determined depending on conditions, including a range that does not cause cell death, a range that does not cause toxicity to a pluripotent stem cell, and the solubility of the PKCβ inhibitor.

For example, the final concentration of the PKCβ inhibitor in a liquid medium may be 15 μM or less, 10 μM or less, 5 UM or less, 3 μM or less, or 1 μM or less.

Meanwhile, the WNT inhibitor may be of one kind or a combination of two or more different kinds thereof. The lower limit of the concentration of the TNKS inhibitor is not particularly limited, and may be determined depending on a range that does not cause cell death.

For example, the final concentration of the WNT inhibitor in a liquid medium may be 90 nM or more, 100 nM or more, 150 nM or more, 200 nM or more, 300 nM or more, 400 nM or more, 500 nM or more, 600 nM or more, 700 nM or more, 800 nM or more, or 900 nM or more. In addition, the final concentration may be, for example, 10 μM or more, 15 μM or more, 18 μM or more, 20 μM or more, or 25 μM or more.

The upper limit of the concentration of the WNT inhibitor is not particularly limited, and may be determined depending on conditions, including a range that does not cause cell death, a range that does not cause toxicity to a pluripotent stem cell, and the solubility of the TNKS inhibitor.

For example, the final concentration of the WNT inhibitor in a liquid medium may be 40 μM or less, 35 μM or less, 30 μM or less, 25 μM or less, 20 μM or less, 15 μM or less, 10 μM or less, 5 μM or less, 3 μM or less, 1.5 μM or less, or 1 μM or less.

A method for adding a PKCβ inhibitor and a TNKS inhibitor is not particularly limited as long as the concentration of each of the PKCβ inhibitor and the TNKS inhibitor in the medium is within the above-described ranges at the start of this step. For example, the concentration may be adjusted by directly adding, to a medium, one or more kinds of PKCβ inhibitor and TNKS inhibitor in a total amount that falls within the above-described concentration ranges.

In adherent culture, the amount of each of the medium and the culture solution is appropriately adjusted depending on the culture vessel to be used. Preferably, the height from the bottom face of the vessel to the liquid surface is desirably 2 mm. For example, in a case where a 300 cm² culture flask is used, the amount of the medium or the culture solution can be, for example, 60 mL. The amount of the medium or the culture solution may be, for example, 1 mL or more, 2 mL or more, 3 mL or more, 4 mL or more, 5 mL or more, 10 mL or more, 20 mL or more, 30 mL or more, 40 mL or more, 60 mL or more, 80 mL or more, or 90 mL or more. In addition, when cells have grown to form a large colony, the amount of the culture solution may be increased to increase the supply of nutrients, and decrease the concentration of the waste product accumulated. For example, the height from the bottom face of the vessel to the liquid surface may be 2.5 mm, 3.0 mm, 3.5 mm, or 4.0 mm. The amount of the medium or the culture solution may be constant or changed during culture.

### (Seeding Density)

In adherent culture, the density (seeding density) of cells to be seeded in a new base material such as in a culture vessel may be appropriately adjusted, considering the state of cells used for seeding, the culture time in this step, the state of cells after culture, and the number of cells required after culture. Without limitation, the range is usually from the lower limit of, for example, 0.5×10³ cells/cm² or 1×10³ cells/cm² to the upper limit of, for example, 5×10⁴ cells/cm² or 10×10⁴ cells/cm². In particular, the seeding density at which cells as a raw material are seeded to start culture is preferably larger to increase the stability of the cells, and is preferably 2×10³ cells/cm² or more, 3×10³ cells/cm² or more, 4×10³ cells/cm² or more, 5×10³ cells/cm² or more, or 6×10³ cells/cm² or more. Further, the seeding density at which cells as a raw material are seeded to start culture is preferably 3×10⁴ cells/cm² or less, 2×10⁴ cells/cm² or less, 1×10⁴ cells/cm² or less, or 0.8×10⁴ cells/cm² or less, because the raw material cells are rare and small in number, and thus, the density, if rendered too high, decreases the culture area usable for seeding, resulting in requiring an unnecessary passage in order to grow the cells.

### (Culture Period)

The culture period in adherent culture can be appropriately adjusted, considering the number of raw material cells, the quality and characteristics such as the adhesion rate and proliferativeness of a raw material cell, the seeding density, and the number of cells necessary to start suspension culture. In addition, the number of passages of adherent culture can be appropriately adjusted in the same manner. Here, a “passage” means that cells subjected to adherent culture are detached, collected, and newly seeded in adherent culture or suspension culture. In addition, “one passage period” refers to a period from the time when cells are seeded to the time when the cells are cultured and collected. The lower limit of one passage period is not particularly limited as long as the period enables the cells seeded to form a colony and grow, and is 2 days, 2.5 days, or 3 days. The upper limit of one passage period may be a period of time in which the cell colony does not become so dense and/or does not expand so widely in a culture vessel as to cause a decrease in the proliferativeness, survival rate, and further, quality such as an undifferentiated property. The upper limit may be for example, 4 days, 4.5 days, 5 days, 5.5 days, 6 days, 6.5 days, 7 days, 7.5 days, 8 days, 8.5 days, 9 days, 9.5 days, or 10 days.

The number of passages in this step is not particularly limited. For example, the number of passages may be 0, 1 or more, 2 or more, or 3 or more. The upper limit is not particularly limited, and is, for example, 5 or less or 4 or less. Before and after a passage, a culture condition (e.g., a vessel, the composition of a medium, the amount of the medium, or the like) may be changed. For example, a passage may be performed in a larger vessel than before the passage, the amount of a medium may be increased, or the seeding density may be lower than before the passage. From the viewpoint of improving productivity, it is preferable that one or more passages are performed in adherent culture, and that a passage is performed in a larger vessel than before the passage, or a passage is performed in a plurality of separate vessels.

### (Culture Conditions)

Culture conditions such as culture temperature, culture time, and CO₂ concentration are not particularly limited. Culture may be performed within the range in conventional methods in the art. For example, the lower limit of the culture temperature may be 20° C. or 35° C., and the upper limit thereof may be 45° C. or 40° C., and preferably, the culture temperature is 37° C. For example, the lower limit of the CO₂ concentration in the gas phase during culture may be 0.5% or more, 1% or more, 2% or more, 3% or more, 4% or more, or 4.5% or more, and the upper limit thereof may be 10% or less or 5.5% or less, and more preferably, the CO₂ concentration is 5%. In this regard, the CO₂ concentration in the gas phase during culture does not need to be constant, and may change and/or be altered during the culture. For example, the lower limit of the O₂ concentration in the gas phase during culture may be 3% or more, or 5% or more, and the upper limit thereof may be 21% or less, or 20% or less, and more preferably, the O₂ concentration is 21%.

In addition, in adherent culture, the medium can be changed with appropriate frequency. The frequency of medium change differs depending on the cell line of cells to be cultured and the cell density, and may be, for example, once or more per 4 days, once or more per 3 days, once or more per 2 days, once or more per 1 day, or 2 times or more per 1 day. The frequency of medium change may be inconstant. For example, the frequency of medium change may be lower during the first half of one passage period, namely, during the period when the cell density is lower, and the frequency of medium change is rendered higher during the second half of the one passage period, namely, during the period when the cell density is higher. Thereby, the survival rate and quality of the cells can be kept. In addition, the medium may be supplied and discharged always continuously by a perfusion mode, not by a batch mode. A method for changing a medium is not particularly limited, and, for example, all or part of the medium may be changed. Because cells adhere to a base material in adherent culture, a specific example may be as follows: a culture supernatant (culture solution) is directly removed from a culture vessel without a particular cell-isolating operation, supplemented with a fresh medium, and allowed to spread over the whole culture surface; and the cells are cultured again. In this regard, a method for changing a medium, and the like are not limited to the above-described frequency or method. An optimal method may be appropriately adopted. The frequency of medium change is not particularly limited. The frequency may be, for example, 0 times, 1 time or more, 2 times or more, or 3 times or more, and may be, for example, 5 times or less, 4 times or less, or 3 times or less.

### (Culture Method)

In adherent culture, the flowing state of the medium during culture is arbitrary. In other words, the adherent culture may be static culture or flow culture.

“Static culture” refers to culturing in a state in which a medium is left statically in a culture vessel. In adherent culture, this static culture is usually adopted. The “flow culture” refers to culturing in a state in which the medium is allowed to flow.

In this adherent culture step, raw material cells after thawing are seeded at a high density, for example, a density of 3×10³ cells/cm² or more, and cultured for one passage period. Then, the cells are once detached, collected, passaged in a vessel having a larger area than in the adherent culture, and preferably further subjected to adherent culture (adherent culture for the second passage). In addition, adherent culture may be further repeated to perform three or more passage cultures. It is usually said that the smaller the number of passages, the higher the quality. Surprisingly, however, the present invention has revealed that culturing for a plurality of passage periods in such a manner makes it possible that higher-quality cells are grown to the number sufficient to perform the subsequent suspension culture step, without imposing excessive stress on the cells.

In this adherent culture step, the cell number obtained through growth can be set arbitrarily. The cell number and state of interest may be appropriately determined according to the cell line to be cultured, the seeding density in the suspension culture, the scale of the suspension culture, the type of the medium, and the culture conditions. For example, the degree of cell growth and the state of cells are not particularly limited. In terms of the occupancy of the culture area in a culture vessel, the lower limit may be 10%, 20%, 30%, 40%, or 50%. Meanwhile, the upper limit can be 100%, 90%, 80%, 70%, or 60%. In particular, in terms of the occupancy of the culture area in a culture vessel, cells are grown preferably so as to be in a state in which the lower limit is 50%, and the upper limit is 80%. In addition, the cell number at the endpoint of the adherent culture step is preferably, but not particularly limited to, 1.5×10⁶ cells or more, 3.0×10⁶ cells or more, 6.0×10⁶ cells or more, 10×10⁶ cells or more, 16×10⁶ cells or more, 20×10⁶ cells or more, 21×10⁶ cells or more, 22×10⁶ cells or more, 25×10⁶ cells or more, 30×10⁶ cells or more, 32×10⁶ cells or more, 35×10⁶ cells or more, 36×10⁶ cells or more, 38×10⁶ cells or more, 40×10⁶ cells or more, 41×10⁶ cells or more, 45×10⁶ cells or more, 48×10⁶ cells or more, or 64×10⁶ cells or more.

This adherent culture preferably increases the number of raw material cells to a certain fold or more. Specifically, it is preferable to be capable of collecting, upon termination of adherent culture, for example, 142 times, 143 times, 145 times, 150 times, 160 times, 170 times, 175 times, 180 times, 200 times, 210 times, 220 times, 230 times, 240 times, 250 times, 260 times, 270 times, or 274 times or more cells compared to the raw material cells seeded.

In this regard, in this adherent culture step, it is possible to isolate a part of the pluripotent stem cells during culture and to confirm the cell number or whether the cells maintain the undifferentiated state. For example, it is possible to confirm whether the undifferentiated state is maintained by measuring the degree of expression of pluripotent stem cell markers expressed in pluripotent stem cells isolated during culture, such as upon passaging. For example, a pluripotent stem cell marker may include Alkaline Phosphatase, NANOG, OCT4, SOX2, TRA-1-60, c-Myc, KLF4, LIN28, SSEA-4, and SSEA-1. Examples of the method for detecting these pluripotent stem cell markers include flow cytometry, as described above.

It can be determined that the undifferentiated state is maintained when the positive rate for the pluripotent stem cell marker in the pluripotent stem cells taken out during culture is preferably 80% or more, more preferably 90% or more, still more preferably 91% or more, still more preferably 92% or more, still more preferably 93% or more, still more preferably 94% or more, still more preferably 95% or more, still more preferably 96% or more, still more preferably 97% or more, still more preferably 98% or more, still more preferably 99% or more, or still more preferably 100%.

In addition, in this step, it is possible to confirm whether the undifferentiated state is maintained by measuring the degree of expression of three germ layer markers (endodermal cell marker, mesodermal cell marker, and ectodermal cell marker) in the pluripotent stem cells isolated during culture. In other words, when the positive rate for all of these endodermal cell marker, mesodermal cell marker, and ectodermal cell marker are preferably 20% or less, more preferably 10% or less, still more preferably 9% or less, still more preferably 8% or less, still more preferably 7% or less, still more preferably 6% or less, still more preferably 5% or less, still more preferably 4% or less, still more preferably 3% or less, still more preferably 2% or less, still more preferably 1% or less, or still more preferably the detection limit or less, it can be determined that the undifferentiated state is maintained. Alternatively, when the degree of expression is a given degree or lower, compared with the expression level of each marker in a cell population after induction of differentiation, the undifferentiated state can be judged to be maintained. Specifically, for example, when the degree of expression is a 10th or less, 50th or less, 100th or less, 200th or less, 300th or less, 400th or less, 500th or less, or 600th or less of the expression level in a cell population after induction of differentiation, the undifferentiated state can be judged to be maintained.

An “endodermal cell marker” refers to a gene specific to endodermal cells. Examples thereof may include SOX17, FOXA2, CXCR4, AFP, GATA4, and EOMES. An endodermal cell forms tissues of organs such as the digestive tract, lung, thyroid, pancreas, and liver, cells of secretory glands opening to the digestive tract, peritoneum, pleura, larynx, auditory tube, trachea, bronchi, urinary tracts (bladder most of the urethra, part of urinary duct), and the like.

A “mesodermal cell marker” refers to a gene specific to mesodermal cells. Examples thereof may include TBXT (BRACHYURY), MESP1, MESP2, FOXF1, HAND1, EVX1, IRX3, CDX2, TBX6, MIXL1, ISLI, SNAI2, FOXC1, and PDGFRα. A mesodermal cell forms body cavities and lining mesothelium, muscle, skeleton, skin dermis, and connective tissue, heart, blood vessels (including vascular endothelium), blood (including blood cells), lymph vessels, spleen, kidneys, ureters, gonads (testis, uterus, gonadal epithelium), and the like.

An “ectodermal cell marker” refers to a gene specific to ectodermal cells. Examples thereof may include FGF5, NESTIN, SOX1, and PAX6. An ectodermal cell forms the epidermis of the skin and epithelium of the terminal urethra in males, hair, nails, skin glands (including mammary and sweat glands), sensory organs (including the terminal epithelium of the oral cavity, pharynx, nose, and rectum, salivary glands), lens, peripheral nervous system, and the like. In addition, part of the ectoderm is invaginated into grooves during development to form the neural tube and also serves as the origin of melanocytes and neurons in the central nervous system, such as the brain and spinal cord.

The degree of expression of these three germ layer markers (endodermal cell marker, mesodermal cell marker, and ectodermal cell marker) can be measured by any detection method in the art. Examples of the method for measuring the expression of three germ layer markers (endodermal cell marker, mesodermal cell marker, and ectodermal cell marker) include, but are not limited to, quantitative real-time PCR analysis, the RNA-Seq method, northern hybridization, or hybridization methods using DNA array. In quantitative real-time PCR analysis, the expression level of the marker gene to be measured is calculated into the relative expression level with respect to the expression level of an internal standard gene, and the expression level of the marker can be evaluated based on the relative expression level. Examples of an internal standard gene include the glyceraldehyde-3-phosphate dehydrogenase (GAPDH) gene and the β-actin (ACTB or bAct) gene.

### (Collection of Cell of Adherent Culture)

A cell after the adherent culture step is collected to be used for the subsequent suspension culture step. In this regard, this collection operation is the same as the operation upon a passage from adherent culture to adherent culture in the adherent culture step. In the step of collecting the pluripotent stem cell subjected to adherent culture, the pluripotent stem cell is separated from the culture solution by a conventional method, and the separated pluripotent stem cell is collected. In this connection, the pluripotent stem cell is preferably collected as a single-state cell by detachment or dispersion treatment from an external matrix or an adjacent pluripotent stem cell. As for the single-state cell, all the cells are not necessarily in a single free state, but a plurality of cells in an adherent state may exist, as long as they are in a state in which a single cell (a cell in a unicellular state) free from an adherent cell colony exists.

An enzymatic detachment agent and/or a chelating agent may be used for unicellularization. The enzymatic detachment agent is not particularly limited. It is possible to use not only a commercially available detachment agent but also an enzyme that enables a cell adhering to a culture vessel to be detached from the culture vessel, and be unicellularized. Examples of the enzymatic detachment agent include trypsin, collagenase, pronase, hyaluronidase, elastase, and commercially available products such as Accutase (registered trademark), Accumax (registered trademark), TrypLE™ Express Enzyme (Life Technologies Japan Ltd.), TrypLE™ Select Enzyme (Life Technologies Japan Ltd.), and Dispase (registered trademark). Examples of a chelating agent that may be used include, but are not particularly limited to, EDTA and EGTA. For example, in the case of using trypsin for unicellularization, the lower limit of the concentration in the solution is not particularly limited as long as the pluripotent stem cell population can be dispersed, and may be, for example, 0.15% by volume, 0.18% by volume, 0.20% by volume, or 0.24% by volume. Meanwhile, the upper limit of the concentration in the solution is not particularly limited as long as the concentration does not cause an effect such as cells per se to be dissolved, and may be, for example, 0.30% by volume, 0.28% by volume, or 0.25% by volume. The treatment time depends on the concentration of trypsin, but the lower limit thereof is not particularly limited as long as the pluripotent stem cell population is well dispersed by the action of trypsin within the time. It may be, for example, 2 minutes, 3 minutes, 5 minutes, 8 minutes, 10 minutes, 12 minutes, or 15 minutes. Meanwhile, the upper limit of treatment time is not particularly limited as long as it does not cause an effect such as cells per se to be dissolved by the action of trypsin, and may be, for example, 30 minutes, 28 minutes, 25 minutes, 22 minutes, 20 minutes, 18 minutes, 15 minutes, 14 minutes, 13 minutes, 12 minutes, 11 minutes, 10 minutes, 8 minutes, 7 minutes, 6 minutes, or 5 minutes. When a commercially available enzymatic detachment agent is used, it may be used at a concentration that allows the cells to be dispersed in a single-cell state, as described in the attached protocol.

For example, when EDTA is used for unicellularization, the lower limit of the concentration in the solution is not particularly limited as long as the pluripotent stem cell population can be dispersed, and is preferably, for example, 0.01 mM, 0.1 mM, or 0.5 mM. Meanwhile, the upper limit of the concentration in the solution is not particularly limited as long as the concentration does not cause an effect such as cells per se to be dissolved, and is preferably 100 mM, 50 mM, 10 mM, or 5 mM. It is preferable to use one or more type of both enzymatic detachment agent and chelating agent for unicellularization. In addition, it is preferable in the unicellularization that the enzymatic detachment agent and the chelating agent which treat a cell contain a ROCK inhibitor. It is known that a pluripotent stem cell in a single-cell state is unstable, and susceptible to cell death. However, allowing the ROCK inhibitor to act simultaneously with the unicellularization can suppress the cell death. The upper limit of the concentration of the ROCK inhibitor may be, for example, 40 μM, 30 μM, or 20 μM, and the lower limit may be, for example, 2 μM, 2.5 μM, 3 μM, 5 μM, 8 μM, 9 μM, or 10 μM. After treatment with the enzymatic detachment agent and/or chelating agent, unicellularization may be promoted by applying a mild stress to an adherent cell colony or an adherent cell colony detached from a base material. This stress-applying treatment is not particularly limited. Conceivable examples include physical stimuli such as: a method in which cells together with a solution are pipetted a plurality of times; a method in which a solution such as a buffer solution is sprayed on adhering cells; a method in which a cell scraper is used; and a method in which an adherent culture vessel is tapped. Further, the cells may be passed through a strainer or mesh, if necessary.

The unicellularized cell may be collected by removing the supernatant containing the detachment agent via standing, centrifugation, or the like. The cell collected may be suspended directly or, if necessary, with a buffer (such as a PBS buffer), physiological saline, or a medium (preferably a medium used in the subsequent step, or a basal medium), and then subjected to the subsequent step. The time to be taken until the cells collected are used in the subsequent step (a passage or a suspension culture step) is not particularly limited. From the viewpoint of maintaining the quality of the cells, the cells are preferably transferred rapidly to the subsequent step. The standby time (the time from the completion of collection to the start of seeding in a passage or the start of seeding in a suspension culture) is, for example, 24 hours or less, 18 hours or less, 12 hours or less, 10 hours or less, 8 hours or less, 6 hours or less, 4 hours or less, 3 hours or less, 2 hours or less, or 1 hour or less. In a case where the standby time is long in relation to the schedule of the steps, the cells collected are desirably stored at low temperature (e.g., 10° C. or less or 5° C. or less). In this regard, during the period of time between the adherent culture step and the suspension culture step, the cells are preferably not freeze-preserved.

In the present invention, performing the adherent culture step under the above-described preferable conditions enables even a small number of pluripotent stem cells to be rendered the cells in numbers suitable for use for suspension culture, and, for example, also enables the culture environment to be controlled by any kind of sensor or the like in a suspension culture in the subsequent step. In addition, in a case where cells having unstable quality are used as a raw material, subjecting the cells to the adherent culture step in the present invention enables the cells to form a cell aggregate efficiently during the suspension culture, and in addition, be inhibited from cell death.

### 1-3-2. Suspension Culture Step

The “suspension culture step” is a step of culturing cells to allow a pluripotent stem cell population to grow while maintaining their undifferentiated state. For suspension culture, an animal cell culture method known in the art may be used. For example, a suspension culture method in which cells are stirred in a liquid medium in a non-cell-adhesive vessel may be used.

### (Cells)

A cell used in this step is a cell cultured and collected in “1-3-1. Adherent Culture Step”, and is a pluripotent stem cell capable of cell aggregation in suspension culture. A pluripotent stem cell used in this step is usually a cell population (pluripotent stem cell population) composed of a plurality of cells. The proportion (percentage) of cells expressing pluripotent stem cell markers (e.g., OCT4, SOX2, or Nanog) and/or cells positive for pluripotent stem cell markers in the cell population is, for example, 90% or more, 91% or more, 92% or more, 93% or more, 94% or more, 95% or more, 96% or more, 97% or more, 98% or more, 99% or more, or 100%.

### (Culture Vessel)

The culture vessel used for suspension culture is not particularly limited, but a culture vessel treated to suppress protein adsorption on the inner surface of the vessel is preferable. In addition, the vessel preferably has a port to which a sensor such as a pH sensor, DO sensor, or temperature sensor can be attached. In addition, the vessel preferably has a port through which gas can be supplied and/or a port through which a medium can be supplied and suctioned. The shape and kind of a culture vessel are not particularly limited. For example, the vessel is in the shape of a dish, flask, cylinder, well, bag, spinner flask vessel, bioreactor including a stirring blade, or the like. For example, a culture vessel usable for a bioreactor may be BioBLU 1c Single-Use Vessel (Eppendorf SE).

The capacity of the culture vessel used may be appropriately selected and is not particularly limited. However, the lower limit of the volume capable of accommodating a medium and of culturing is preferably 1 mL, 2 mL, 4 mL, 10 mL, 20 mL, 30 mL, 50 mL, 100 mL, or 200 mL, and the upper limit thereof is preferably 1000 L, 100 L, 50 L, 20 L, 10 L, 5 L, 3 L, 1 L, or 500 mL. However, to culture a large number of cells for the purpose of producing a cell stock, the scale of culture is preferably large, and the amount of culture solution for one passage period during which the cells for use for preparation of a cell stock are collected is particularly preferably 100 mL or more. When a stirring blade-type reactor of arbitrary capacity is used, the capacity may be within the range of the working volume specified by each manufacturer of the reactor.

As used herein, the volume of the medium that is being accommodated in the culture vessel and used for cell culture is referred to as the culture volume or the amount of the culture solution.

### (Medium)

The medium used for suspension culture is a medium containing a basal medium, as described above in “1-2. Definitions of Terms,” preferably containing a ROCK inhibitor. A medium containing a ROCK inhibitor enables a cell aggregate to have higher strength to a shear stimulus, and to undergo suspension culture more stably. In addition, the medium used for suspension culture is preferably a medium containing a PKCβ inhibitor and/or a WNT inhibitor. A medium containing a PKCβ inhibitor and/or a WNT inhibitor can further suppress the spontaneous differentiation of pluripotent stem cells and the deterioration of the quality, and improve the quality in some cases. In addition, a medium used in the present invention is preferably a liquid medium containing at least one selected from the group consisting of L-ascorbic acid, insulin, transferrin, selenium, and sodium hydrogen carbonate. In addition, the liquid medium preferably contains at least one growth factor, and the liquid medium more preferably contains FGF2 and/or TGF-β1 as the growth factor. The medium is particularly preferably a serum-free DMEM/F12 medium containing L-ascorbic acid, insulin, transferrin, selenium, sodium hydrogen carbonate, FGF2, and TGF-β1.

In addition, in the present invention, the medium in this step is preferably changed by a perfusion mode in which the medium is perfused. Changing the medium by a perfusion mode enables the culture environment to be controlled continuously. In a case where the perfusion mode is used as a mode of medium change in this step, the culture additive composition of the medium used in this step may not be constant. Specifically, the culture additive composition of the medium at the start of culture in this step may be different from the culture additive composition of the medium used for medium change by the perfusion mode during culture in this step. A plurality of types of media may be used for medium change by the perfusion mode during culture in this step, and the medium used for medium change by the perfusion mode may be switched to one with a different culture additive composition at an arbitrary time point during culture. In addition, the culture additive composition of a liquid medium used in perfusion may be changed during culture. Changing the culture additive composition of the medium as described above makes it possible to continuously control the concentration of an arbitrary culture additive or medium component in the culture system in accordance with various medium perfusion schemes (the amount of medium perfused per unit time, or the like), thereby achieving an appropriate concentration transition. In addition, in the perfusion mode, the amount of medium perfused is preferably increased from an arbitrary time point in accordance with the growth of the cells. The more preferable amount of medium perfused is as below-described.

The lower limit of the ROCK inhibitor concentration in the medium in this step may be, for example, 0 μM, 1 μM, 2 μM, 2.5 μM, 3 μM, 5 μM, 7 μM, 8 μM, 9 μM, or 10 μM as the final concentration in the liquid medium at the start of culture in this step.

The upper limit of the concentration of the ROCK inhibitor in the liquid medium at the start of culture in this step is not particularly limited, and may be determined depending on conditions, including a range that does not cause cell death, a range that does not cause deviation from the undifferentiated state, and the solubility of the ROCK inhibitor.

For example, the upper limit of the ROCK inhibitor concentration in the liquid medium in the perfusion mode of this step may be 50 μM, 40 μM, 30 μM, or 20 μM as the final concentration in the liquid medium at the start of culture.

Without particular limitation, in a case where a ROCK inhibitor is used, the ROCK inhibitor concentration in the liquid medium used for medium change by the perfusion mode in this step is preferably lower than the ROCK inhibitor concentration in the liquid medium at the start of culture in this step.

The upper limit of the concentration of the ROCK inhibitor as the final concentration in the liquid medium used for medium change by the perfusion mode in this step is not particularly limited, and may be determined depending on conditions, including a range that does not cause cell death, a range that does not cause deviation from the undifferentiated state, and the solubility of the ROCK inhibitor.

For example, the upper limit of the concentration of the ROCK inhibitor may be 50 μM, 40 μM, 30 μM, or 20 μM as the final concentration in the liquid medium used for medium change by the perfusion mode in this step.

For example, the lower limit of the concentration of the ROCK inhibitor may be 0 μM, 1 μM, 2 μM, 2.5 μM, 3 μM, 5 M, 7 μM, 8 μM, 9 μM, or 10 μM as the final concentration in the liquid medium used for medium change by the perfusion mode in this step.

The method for adding the ROCK inhibitor is not particularly limited as long as the concentration of the ROCK inhibitor in the medium is within the above-described range. For example, the concentration may be adjusted by directly adding the ROCK inhibitor to the medium in a total amount that falls within the concentration range or by adding a ROCK inhibitor solution diluted with a different solvent and mixing with the medium.

In addition, the lower limit of the concentration of the PKCβ inhibitor in the culture medium in this step may be, for example, 0 μM, 0.2 μM, 0.4 μM, 0.6 μM, 0.8 μM, 0.9 μM, 1 μM, or 1.1 μM as the final concentration in the liquid medium at the start of culture in this step.

The upper limit of the concentration of the PKCβ inhibitor in the liquid medium at the start of culture in this step is not particularly limited, and may be determined depending on conditions, including a range that does not cause cell death, a range that does not cause deviation from the undifferentiated state, and the solubility of the PKCβ inhibitor.

For example, the upper limit of the final concentration in the liquid medium at the start of culture in this step may be 10 μM, 5 μM, 2 μM, 1.5 μM, or 1 μM.

In this regard, when the cells collected in the adherent culture are seeded in a suspension culture to start culture, the PKCβ inhibitor is preferably not added until the cells seeded form a cell aggregate. A PKCβ inhibitor will undesirably induce cell death slightly to a pluripotent stem cell in a unicellular state immediately after adherent culture.

Without particular limitation, in a case where a PKCβ inhibitor is used, the concentration of the PKCβ inhibitor in the liquid medium used for medium change by the perfusion mode in this step is preferably equal to or higher than the concentration of the PKCβ inhibitor in the liquid medium at the start of culture in this step.

The upper limit of the concentration of the PKCβ inhibitor as the final concentration in the liquid medium used for medium change by the perfusion mode in this step is not particularly limited, and may be determined depending on conditions, including a range that does not cause cell death, a range that does not cause deviation from the undifferentiated state, and the solubility of the PKCβ inhibitor.

For example, the upper limit of the concentration of the PKCβ inhibitor may be 10 μM, 5 μM, 2 μM, 1.5 μM, or 1 μM as the final concentration in the liquid medium used for medium change by the perfusion mode in this step.

For example, the lower limit of the concentration of the PKCβ inhibitor may be 0 μM, 0.2 μM, 0.4 μM, 0.6 μM, 0.8 μM, 0.9 μM, 1 μM, or 1.1 μM as the final concentration in the liquid medium used for medium change by the perfusion mode in this step.

The method for adding the PKCβ inhibitor is not particularly limited as long as the concentration of the PKCβ inhibitor in the medium is within the above-described range. For example, the concentration may be adjusted by directly adding the PKCβ inhibitor to the medium in a total amount that falls within the concentration range or by adding a PKCβ inhibitor solution diluted with a different solvent and mixing with the medium.

In addition, the lower limit of the concentration of the WNT inhibitor in the medium in this step may be, for example, 0 μM, 1 μM, 2 μM, 3 μM, 5 μM, 7 μM, 10 μM, 15 μM, 18 μM, or 20 μM as the final concentration in the liquid medium at the start of culture in this step.

The upper limit of the concentration of the WNT inhibitor in the liquid medium at the start of culture in this step is not particularly limited, and may be determined depending on conditions, including a range that does not cause cell death, a range that does not cause deviation from the undifferentiated state, and the solubility of the WNT inhibitor.

For example, the upper limit of the final concentration in the liquid medium at the start of culture in this step may be 50 μM, 40 μM, 30 μM, 25 μM, or 20 μM.

Without particular limitation, in a case where a WNT inhibitor is used, the concentration of the WNT inhibitor in the liquid medium used for medium change by the perfusion mode in this step is preferably equal to or higher than the concentration of the WNT inhibitor in the liquid medium at the start of culture in this step.

The upper limit of the WNT inhibitor concentration as the final concentration in the liquid medium used for medium change by the perfusion mode in this step is not particularly limited, and may be determined depending on conditions, including a range that does not cause cell death, a range that does not cause deviation from the undifferentiated state, and the solubility of the WNT inhibitor.

For example, the upper limit of the concentration of the WNT inhibitor may be 50 μM, 40 μM, 30 μM, 25 μM, or 20 μM as the final concentration in the liquid medium used for medium change by the perfusion mode in this step.

For example, the lower limit of the concentration of the WNT inhibitor may be 0 μM, 1 μM, 2 μM, 3 μM, 5 μM, 7 μM, 10 μM, 15 μM, 18 μM, or 20 μM as the final concentration in the liquid medium used for medium change by the perfusion mode in this step.

The method for adding the WNT inhibitor is not particularly limited as long as the concentration of the WNT inhibitor in the medium is within the above-described range. For example, the concentration may be adjusted by directly adding the WNT inhibitor to the medium in a total amount that falls within the concentration range or by adding a WNT inhibitor solution diluted with a different solvent and mixing with the medium.

In addition, the medium used for perfusion in the present invention is preferably at a cold-storage temperature. For example, the medium is preferably retained in a cold-storage state until immediately before use for culture by perfusion. The cold storage makes it possible to suppress the decomposition and degradation of a protein component such as a growth factor in the medium. The lower limit of the cold-storage temperature may be, for example, a temperature at which the medium is not frozen, and is preferably 0° C., 1° C., 2° C., 3° C., or 4° C. The upper limit is preferably, for example, 12° C., 10° C., 8° C., 7° C., 6° C., 5° C., or 4° C.

In the present invention, the volume of carbon dioxide gas supplied may be reduced in line with the progress of culture. In other words, the dissolved carbon dioxide gas concentration in the culture solution may be reduced. Meanwhile, when performing medium change by the perfusion described above, if the dissolved carbon dioxide gas concentration in the medium used for perfusion is higher than the dissolved carbon dioxide gas concentration in the culture solution, the dissolved carbon dioxide gas concentration in the culture solution would increase. Therefore, the dissolved carbon dioxide gas concentration in the medium used for perfusion is preferably lower than the dissolved carbon dioxide gas concentration in the culture solution.

### (Seeding Density)

In suspension culture, the density of cells to be seeded in a new medium (seeding density) may be appropriately adjusted, considering the state of cells used for seeding, the cell yield in the adherent culture step as the preceding step, the culture time in this step, and the number of cells required after culture. Without limitation, the lower limit of the seeding density may usually be a density at which cells can form a cell aggregate, and the state of the cells is not unstable. The density may be, for example, 0.01×10⁵ cells/mL, 0.1×10⁵ cells/mL, 0.5×10⁵ cells/mL, 1×10⁵ cells/mL, 1.25×10⁵ cells/mL, 1.5×10⁵ cells/mL, or 2×10⁵ cells/mL. The upper limit may be a cell density that does not cause excessive aggregation or injury to cells, and does not cause the rapid consumption of the medium component. The upper limit may be, for example, 100×10⁵ cells/mL, 50×10⁵ cells/mL, 10×10⁵ cells/mL, 8×10⁵ cells/mL, 6×10⁵ cells/mL, 4×10⁵ cells/mL, 2×10⁵ cells/mL, 1.5×10⁵ cells/mL, or 1.4×10⁵ cells/mL. The growth efficiency in the early stage of culture depends on the seeding density. Thus, for example, the lower limit of the seeding density is particularly preferably 1×10⁵ cells/mL, and the upper limit thereof is particularly preferably 2×10⁵ cells/mL. In addition, in a case where passages are performed in the suspension culture step, the seeding density may be changed in each passage period. For example, the seeding density may be increased gradually from passage to passage.

### (Culture Conditions)

Culture conditions such as culture temperature, culture time, and oxygen concentration are not particularly limited. Culture may be performed within the range in conventional methods in the art. For example, the lower limit of the culture temperature may be 20° C. or 35° C., and the upper limit thereof may be 45° C. or 40° C., but preferably, the culture temperature is 37° C. In addition, the culture time can be appropriately adjusted depending on the desired number of cells to be acquired as a cell stock, the proliferativeness of the cell line, the state of the cells collected in adherent culture, or the like. For example, a period of 24 hours, 48 hours, 60 hours, 72 hours, or 75 hours as the lower limit per one passage period enables the cells to grow sufficiently. A period of 168 hours, 144 hours, 120 hours, 96 hours, 84 hours, or 78 hours as the upper limit enables the cells to be cultured without causing the survival rate to be decreased, for example, by the excessive enlargement of a cell aggregate, and without causing a decrease in a quality such as an undifferentiated property. For example, the lower limit of the oxygen concentration during culture may be 3% or 5%, and the upper limit thereof may be 21% or 20%, and more preferably, the oxygen concentration during culture is 21%.

### (Culture Method)

In the suspension culture in this step, any method for supplying gas may be used, and a standard technique used in general culture methods may be used. For example, without limitation, the gas supplied may be supplied by aerating the liquid surface of the culture solution. Alternatively, the culture solution may be bubbled with gas using a sparger. Alternatively, a desired gas is allowed to fully surround the culture solution, and suppled in a naturally diffusive manner. In this step, a method for aerating the liquid surface of the culture solution can be appropriately used.

Regarding the amount of gas supplied, when cells are cultured in a culture apparatus such as an incubator, the amount of gas supplied may be an amount sufficient to fill the inside of the apparatus. When cells are cultured in a vessel such as a bioreactor, aeration is performed via a gas supplying port of the vessel, and the amount of gas supplied may be appropriately determined considering the culture volume, the surface area of the culture solution, the gas requirement of the culture cells, the speed of gas movement in the culture solution, and the like. As an example, in the case of culture with a culture solution amount of 320 mL using a BioBLU 1c Single-Use Vessel (Eppendorf SE), the suitable amount of gas supplied is 0.1 L/min, 0.2 L/min, or 0.3 L/min. When the culture solution amount is increased relative to the culture solution amount described above, the amount of gas supplied may be increased. When the culture solution amount is decreased relative to the culture solution amount described above, the amount of gas supplied may be decreased.

In this suspension culture step, the concentration of carbon dioxide gas supplied to the liquid medium can be changed. In a general cell culture method, the concentration of each gas component, such as carbon dioxide gas, is constant throughout the culture. However, it must be changed appropriately to respond to the sequential changes in the cell state and the medium environment during culture. In the present invention, changing the concentration of the supply carbon dioxide gas in the range of 10% to 0% in accordance with the progress of the culture in this step can maintain the culture environment appropriately, and produce a cell stock high in a quality such as the survival rate, and thus, is preferable. In this step, the lower limit of the carbon dioxide gas concentration in the gas supplied is preferably 0%, 0.5%, or 1%, and the upper limit is preferably 10%, 9%, 8%, 7%, 6%, or 5%. The amount of carbon dioxide gas supplied in the liquid medium is obtained by multiplying the carbon dioxide gas concentration in the gas supplied by the supply amount of the gas supplied. In other words, as the method for changing the amount of carbon dioxide gas supplied in the culture solution, a method for changing the carbon dioxide gas concentration in the gas supplied, a method for changing the supply amount of the gas supplied containing carbon dioxide gas, or a method in which both methods are combined may be used.

It is considered that as the culture progresses, the cells grow, and the total volume of oxygen consumed and carbon dioxide released by the cells themselves increases. Therefore, the culture environment can be more uniformly controlled by altering the supply amount of carbon dioxide gas from the outside. In addition, the influence of metabolites other than carbon dioxide discharged by the cells in line with the progress of culture on the culture environment can be controlled by reducing the supply amount of carbon dioxide gas supplied. In other words, it is possible to reduce the supply amount of carbon dioxide gas supplied in line with the progress of culture in this step. For example, it is preferable to reduce the carbon dioxide gas concentration in line with the progress in culture when the supply amount of the gas supplied is constant. The amount of carbon dioxide gas supplied does not need to be reduced monotonously, and may be gradually reduced by increasing and decreasing the carbon dioxide gas concentration to adjust the balance.

In addition, the carbon dioxide gas concentration can be decreased stepwise. The carbon dioxide gas concentration can be decreased, for example, to a first range within a first period of time from the start of decrease, and decreased to a second range within the second period of time. Specifically, for example, the concentration can be decreased to the range of 0% to 2.5% within 1.5 days from the start of decrease, and decreased to the range of 0% to 1% within 2 days from the start of decrease.

The amount of carbon dioxide gas supplied to the liquid medium may be altered based on one or more indicators. Examples of indicators for decreasing the carbon dioxide gas concentration in line with the progress of culture include pH, cell density, lactic acid concentration, and lactic acid production rate of cells. These indicators may be selected independently of or in association with the culture variables used to control the amount of medium perfused. The amount of carbon dioxide gas supplied may be reduced proportionally or negatively proportionally to one of or a combination of a plurality of these indicators. Therefore, the below-described formulas for the culture variables may also be used for these indicators.

In this case, the sign of the correction coefficient M is usually reversed from that used in the culture variables. For example, in a case where the cell density, cell density increasing rate, cell number, or cell aggregate size or volume is used as an indicator, a negative M value is used because, generally, it is preferable to decrease the carbon dioxide gas concentration as these variables increase. Meanwhile, for example, in a case where pH is used instead of the cell density as an indicator, a positive M value is used because, generally, it is preferable to decrease the carbon dioxide gas concentration as the pH decreases.

For example, the pH of the culture solution may be an indicator. In this case, the amount of carbon dioxide gas supplied can be altered (especially decreased) by changing the carbon dioxide gas concentration in the gas supplied to suppress the pH decrease. Specifically, for example, the carbon dioxide gas concentration in the gas supplied may be set to be proportional to the pH value.

The timing of starting to decrease the carbon dioxide gas concentration is arbitrary. In addition, unlike the timing of starting the below-described medium perfusion, the timing of starting to decrease the carbon dioxide gas concentration may be before cells form cell aggregates, and the decrease of the carbon dioxide gas concentration may start at the start of culture. The timing of start of a decrease in the carbon dioxide gas concentration can be, for example, when the pH of the culture solution has fallen below any criterion. The pH as the criterion can be, for example, 7.25, 7.24, 7.23, 7.22, 7.21, 7.20, 7.19, 7.18, 7.17, 7.16, 7.15, 7.14, 7.13, 7.12, 7.11, 7.10, 7.09, 7.08, 7.07, 7.06, 7.05, 7.04, 7.03, 7.02, 7.01, 7.00, 6.99, 6.98, 6.97, 6.96, or 6.95. As below-described, in the case of starting to culture cells in the unicellular state, it is not preferable to perform liquid medium perfusion before the formation of cell aggregates. Hence, adjusting the carbon dioxide gas concentration by adjusting the carbon dioxide gas concentration enables the growth ability (e.g., the specific growth rate), survival rate, and undifferentiated property of the cells to be maintained and improved even during a period when the culture environment cannot be controlled by perfusion.

In the suspension culture of this step, the medium during culture is in a flowing state. The “flow culture” refers to culturing under conditions that allow the medium to flow. In the case of flow culture, a method that allows the medium to flow so as to promote aggregation of cells seeded in a unicellular state, and so as suppress excessive aggregation of the cells is preferable. For example, as such a culture method, the rotation culture method, the rocking culture method, the stirring culture method, and any combination thereof are included. In the present invention, it is preferable that the suspension culture in this step is performed by a stirring culture method, that is, that this step is a suspension stirring culture. In addition, a microcarrier or the like is preferably not used from the viewpoints of scale-up and the simplicity of a step.

The “rotation culture method” (including the shaking culture method) refers to a method for performing culture under conditions that allow a medium to flow such that cells gather at one point due to the stress (force) (centrifugal force, centripetal force) caused by rotational flow. Specifically, culture is performed by rotating a culture vessel accommodating a medium containing cells along a substantially horizontal plane in a closed orbit such as a circle, an ellipse, a deformed circle, a deformed ellipse, or the like.

The rotation speed is not particularly limited, but the lower limit thereof may be 1 rpm, 10 rpm, 50 rpm, 60 rpm, 70 rpm, 80 rpm, 83 rpm, 85 rpm, or 90 rpm. Meanwhile, the upper limit thereof may be 200 rpm, 150 rpm, 120 rpm, 115 rpm, 110 rpm, 105 rpm, 100 rpm, 95 rpm, or 90 rpm. The amplitude of a shaker used for rotation culture is not particularly limited, but the lower limit thereof may be, for example, 1 mm, 10 mm, 20 mm, or 25 mm. Meanwhile, the upper limit thereof may be, for example, 200 mm, 100 mm, 50 mm, 30 mm, or 25 mm. The radius of rotation during rotation culture is not particularly limited, but the amplitude is preferably set within the above-described range. The lower limit of the radius of rotation may be, for example, 5 mm or 10 mm, and the upper limit of the radius of rotation may be, for example, 100 mm or 50 mm. In particular, when the present method is used as the below-described method for producing a cell aggregate or the like, it is preferable to set the rotation condition within the above-described range since a uniform cell aggregate having an appropriate size may be easily produced.

The “rocking culture method” refers to a method for performing culture under conditions that impart a rocking flow to a medium by linear reciprocating motion, such as rocking stirring. Specifically, culture is performed by rocking a culture vessel accommodating a medium containing cells along a plane substantially perpendicular to the horizontal plane. The rocking rate is not particularly limited. For example, given that one round trip is regarded as one time, the lower limit thereof may be two times, four times, six times, eight times, or ten times of rocking per minute. Meanwhile, the upper limit thereof may be 15 times, 20 times, 25 times, or 50 times of rocking per minute. During rocking, it is preferable to give the culture vessel a slight angle, namely a rocking angle, relative to the vertical plane. The rocking angle is not particularly limited. For example, the lower limit thereof may be 0.1°, 2°, 4°, 6°, or 8°, and the upper limit thereof may be 20°, 18°, 15°, 12°, or 10°. When the present method is used as the below-described method for producing a cell aggregate or the like, it is preferable to set the rocking condition within the above-described range since a cell aggregate having an appropriate size may be easily produced.

Further, culture may also be performed while stirring by a combination of the above-described rotation and rocking motions.

The “stirring culture method” refers to a method for culturing while stirring a culture solution with a stirring blade or stirrer and under conditions that allow cells and/or cell aggregates and the like to be dispersed in the culture solution. When the medium during culture is made fluid by stirring with a stirring blade, although not particularly limited, the lower limit of the stirring rate is preferably 1 rpm, 5 rpm, 10 rpm, 20 rpm, 30 rpm, 40 rpm, 50 rpm, 60 rpm, 65 rpm, 66 rpm, 67 rpm, 68 rpm, 70 rpm, 75 rpm, 80 rpm, 90 rpm, 100 rpm, 110 rpm, 120 rpm, or 130 rpm, and the upper limit thereof is preferably 200 rpm, 190 rpm, 180 rpm, 170 rpm, 160 μm, 150 rpm, 140 rpm, 130 rpm, 120 rpm, 110 rpm, 100 rpm, 90 rpm, 80 rpm, 79 rpm, 78 rpm, 77 rpm, 76 rpm, 75 rpm, 70 rpm, 60 rpm, 50 rpm, 40 rpm, or 30 pm.

In the “stirring culture method,” which is a suspension culture in a stirring mode using a reactor with a stirring blade or the like, it is preferable to control the shear stress applied to the cells during culture. Animal cells, including pluripotent stem cells, are generally more susceptible to physical stress than other types of cells. Therefore, when excessive shear stress is applied to cells during stirring culture, cells might be physically damaged, their growth ability is reduced, and the cell aggregate collapses, causing the cells to die, and in the case of pluripotent stem cells, they cannot maintain their undifferentiated property. Meanwhile, when the shear stress applied to cells during the stirring culture is too small, the cells undergo excessive aggregation in some cases.

Shear stress applied to cells in stirring culture is not limited, but depends on, for example, the blade tip speed. The blade tip speed is the circumferential speed of the tip of the stirring blade and can be obtained as the following formula: Blade diameter [m]×Circumference ratio×Rotational rate [rps]=Blade tip speed [m/s]. In a case where a plurality of blade diameters are obtained because of the tip shape of the stirring blade, the largest diameter may be used.

The blade tip speed is not particularly limited, but the lower limit thereof is preferably 0.05 m/s, 0.08 m/s, 0.10 m/s, 0.13 m/s, 0.17 m/s, 0.20 m/s, 0.23 m/s, 0.25 m/s, or 0.30 m/s. Setting the blade tip speed within this range can suppress excessive aggregation of cells as well as keep pluripotent stem cells undifferentiated.

Further, the blade tip speed is not particularly limited. The upper limit thereof is preferably 1.37 m/s, 1.00 m/s, 0.84 m/s, 0.50 m/s, 0.42 m/s, 0.34 m/s, or 0.30 m/s. Setting the blade tip speed within this range can stabilize the flowing state of the medium in the culture system as well as maintain pluripotent stem cells undifferentiated. In addition, in a case where cells in a unicellular state are seeded and subjected to stirring culture, a small-size cell aggregate preferable in terms of nutrient supply or the like can be formed.

In addition, the blade tip speed is optionally not constant in the stirring culture, and may be changed during the culture. A cell aggregate becomes larger as the cells grow, and hence, for example, the stirring rate is preferably decreased as the size of the cell aggregate is increased. For example, the blade tip speed may be changed between the first half and second half of the culture, or the blade tip speed may be changed every 24 hours during the culture. Changing the blade tip speed during culture in such a manner makes it possible, in some cases, that the damage caused to cells by a shear stress applied to the cell aggregate is maintained at a lower level.

In addition, without particular limitation, when the scale of culture is changed in the stirring culture, the rotational rate of the stirring blade can be determined using the equation with a constant Pv. Pv is a per-unit-volume power required for stirring. Using the same Pv value enables stirring culture to be performed in the same manner between different scales. The equation with a constant Pv can be represented as follows: Rotational rate per unit time [rpm or rps]×(Blade diameter [m])^{2/3}=Constant.

It is preferable to start the medium change by the perfusion mode in a state in which the cells seeded in the culture solution adhere to each other and form cell aggregates. When medium change is performed by the perfusion mode, for example, it is preferable to start perfusion in the perfusion mode after the formation of cell aggregates. As a result, cell aggregates can be retained in the culture solution upon medium change using a filter that removes only the medium but not cells from the culture solution, which will be described later. All the cells in the culture solution do not need to form cell aggregates, and cells in a unicellular state may exist. Cells in a unicellular state at the start of perfusion may form cell aggregates under medium perfusion. The proportion of the number of cells forming cell aggregates with respect to the seeded cell number at the start of medium perfusion is not particularly limited, but the lower limit thereof is preferably 10%, 20%, 30%, 40%, 50%, 60%, 70%, or 80%, and the upper limit thereof is preferably 300%, 200%, 150%, 140%, 130%, 120%, 110%, 100%, or 90%. In general, some of the cells seeded in the suspension culture die, and the cell number temporarily decreases relative to the seeding concentration. It is preferable that the proportion of this decrease is lower, that the start of growth after seeding is earlier, and that the growth rate is higher. When medium change is performed by the perfusion mode, there is a concern that the excessively high proportion of the number of cells forming cell aggregates with respect to the seeded cell number at the start of perfusion may result in progressing of nutrient depletion before the start of perfusion and cells being adversely affected. Therefore, the proportion is preferably not excessively high. For this reason, it is preferable to set the lower limit to 100% for the range of the proportion of the number of cells forming cell aggregates with respect to the seeded cell number.

In addition, in a case where a perfusion mode is used as a mode of medium change in this step, the timing to start the medium change by the perfusion mode may be arbitrarily set considering the number of cells to be seeded, the efficiency of forming cell aggregates seeded, proliferativeness of cells, and the like as long as the cells in the culture solution are in a state of adhering to each other and forming cell aggregates. Without particular limitation, the timing to start perfusion is preferably, for example, 72 hours or later, 60 hours or later, 48 hours or later, 42 hours or later, 36 hours or later, 30 hours or later, 24 hours or later, 18 hours or later, or 12 hours or later after seeding cells and starting culture.

The amount of medium perfused per unit time at the start of perfusion (herein sometimes referred to as “reference perfusion rate”) may be determined arbitrarily. The reference perfusion rate is an amount of medium perfused obtained by multiplying the amount of medium perfused for substituting 100% of the medium volume in a certain period by a start coefficient based on the culture conditions at the start of culture. In this connection, the length of the certain period is not particularly limited, and may be, for example, 1 hour, 3 hours, 5 hours, 6 hours, 9 hours, 12 hours, 15 hours, 18 hours, 20 hours, 24 hours, 30 hours, 36 hours, 42 hours, 48 hours, 60 hours, or 72 hours. Although not particularly limited, for example, when the certain period is 24 hours, the reference perfusion rate can be set based on a value obtained by multiplying the culture volume by the proportion of the unit time length with respect to 24 hours. Specifically, for example, when the unit time length is 1 hour and the certain period is 24 hours, the reference perfusion rate is based on a value obtained by dividing the culture volume by 24.

A value that may be determined as the reference perfusion rate is obtained by multiplying the above value, the amount of medium perfused per unit time at the start of culture, by an appropriate value according to the culture conditions at the start of culture as the start coefficient, such as the cell seeding density, the proportion of the number of cells forming cell aggregates with respect to the seeded cell number at the start of perfusion, or the like. The lower limit of the start coefficient is preferably 0.1, 0.5, 0.6, 0.7, 0.8, 0.9, or 1.0, and the upper limit thereof is preferably 2.0, 1.9, 1.8, 1.7, 1.6, 1.5, 1.4, 1.3, 1.2, 1.1, or 1.0.

The start coefficient can be appropriately set according to the purpose and conditions. For example, with a start coefficient of a particular condition set at 1.0, a value based on the magnitude of deviation from said particular condition (e.g., the proportion of the actual cell density with respect to the certain cell seeding density or the ratio of an actual proportion of the number of cells forming cell aggregates with respect to the seeded cell number at the start of perfusion, with respect to a certain proportion of the number of cells forming cell aggregates with respect to the seeded cell number at the start of perfusion) may be used as the start coefficient. Examples of the particular condition include a standard culture condition for using allogeneic cells and a culture condition recommended by the cell provider.

After starting perfusion, the timing to start the control of the amount of medium perfused per unit time may be set arbitrarily. The control of the amount of medium perfused per unit time may be started at the start of perfusion or 6 hours or later, 12 hours or later, 18 hours or later, 24 hours or later, 30 hours or later, 36 hours or later, 42 hours or later, 48 hours or later, 54 hours or later, 60 hours or later, 66 hours or later, 72 hours or later after medium perfusion. It is preferable to start the control of the amount of medium perfused before the culture environment, such as the lactic acid concentration and pH, changes remarkably, to an extent that cells begin to be affected adversely.

The amount of medium perfused per unit time of medium change by the perfusion mode (herein sometimes referred to as “variable perfusion rate”) preferably has a lower limit of 0.1%, 1%, 3%, 5%, 10%, 20%, 30%, 40%, or 50% and an upper limit of 100%, 90%, 80%, 70%, 60%, or 50% with respect to the culture volume. In this case, the “amount of medium perfused per unit time” refers to the amount of medium perfused per hour.

It is preferable to control the variable perfusion rate in line with the progress of culture in the above-described range. In other words, it is preferable to control the variable perfusion rate in a range of 1% to 100% of the culture volume based on the reference perfusion rate and the culture variable in a particular culture condition in the suspension culture step. As long as the variable perfusion rate is controlled by the method according to the present invention, its transition is arbitrary. For example, perfusion may be performed at a constant amount in unit time, or the amount of medium perfused may be decreased in the first half of the unit time and increased in the second half of the unit time. Intermittent perfusion may be performed by stopping perfusion only for a part of the unit time. Preferably, the control of the variable perfusion rate in line with the progress of culture is based on one or more culture variables. The culture variable is a variable based on a particular culture condition. Specific examples of the culture variable include the cell density, the cell number, the cell aggregate size or volume, the amount of lactic acid in the culture solution, pH in the culture solution, and the amount of lactic acid produced by metabolism per cell per unit time. In addition, the cell density increasing rate, which is the proportion of a cell density relative to a cell density at the start of controlling the amount of medium perfused, may be set as a culture variable. The cell aggregate volume increasing rate, which is the proportion of a cell aggregate volume relative to a cell aggregate volume at the start of controlling the amount of medium perfused, may also be set as a culture variable. For example, when one of the culture variables is the cell density increasing rate, the amount of medium perfused may be controlled by increasing the variable perfusion rate based on the increase of the cell density increasing rate. In addition, for example, when one of the culture variables is the cell aggregate volume increasing rate, the amount of medium perfused may be controlled by increasing the variable perfusion rate based on the increase of the cell aggregate volume increasing rate.

The amount of medium perfused per unit time may be changed continuously or intermittently in accordance with the changes in one or more of these culture variables. For example, the amount of medium perfused per unit time may be controlled to be proportional to each of the one or more culture variables. In other words, if it is based on a plurality of culture variables, the amount of medium perfused may be controlled such that a proportional relationship is established for each culture variable if the other culture variables are assumed to be constants.

For example, when the cell density is a culture variable, the amount of medium perfused per unit time may be increased in line with the increase of the cell density. For example, it may be increased proportionally with the increase of the cell density. When pH is a culture variable, the amount of medium perfused per unit time may be controlled to suppress the decrease in pH. Suppressing the decrease in pH means maintaining or slightly increasing pH to prevent a pH decrease, or decelerating the pH decreasing rate. It is possible to suppress the decrease in pH by increasing the amount of medium perfused and/or decreasing the amount of carbon dioxide gas supplied to the medium as described later. Therefore, the pH decrease may be suppressed by, for example, increasing the amount of medium perfused per unit time based on the decrease in pH. In addition, a decrease in the pH can also be suppressed by adding a substance, such as caustic soda or sodium bicarbonate, that has a pH equal to or higher than the pH of the culture solution. However, a change in the osmotic pressure of the culture medium will adversely affect the cells, and it is difficult to decrease the concentration of lactic acid, namely, a waste product that adversely affects the cell. Adjusting the amount of medium perfused can achieve both the suppression of a decrease in the pH and the suppression of the lactic acid concentration, and thus, is preferable.

Hereinafter, the control of the amount of medium perfused will be described using mathematical formulas, taking as an example the case where the cell density or the like is used as the culture variable. However, this is only an example, and even when other information is used as the culture variable, the amount of medium perfused can be controlled in the same manner.

For example, in a case where one of the culture variables is the cell density increasing rate, given that the amount of medium perfused at the beginning of controlling the amount of medium perfused per unit time (that is, reference perfusion rate) is F₀, the cell density at that time is C₀, and the cell density at an arbitrary time point in each subsequent culture time is C, the amount of medium perfused per unit time at the arbitrary time point (that is, variable perfusion rate) F can be expressed by the following Formula 1 that is proportional to the cell density increasing rate.

$$F = {F}_{0} \times \frac{C}{{C}_{0}} \qquad [ Formula 1 ]$$

The value of C may be a value previously assumed in accordance with the characteristics of the cell or a preliminary study, or reflect a value measured during culture. It may be switched during culture, such as by using the assumed value as C in the first half of culture and applying the measured value as C in the second half of culture. Without particular limitation, in a case where an environment for an optimal culture solution can be kept optimal by a method according to the present invention, thus enabling a culture, the specific growth rate of a pluripotent stem cell can be assumed to be 0.6 day⁻¹ or more, 0.7 day⁻¹ or more, 0.8 day⁻¹ or more, or 0.9 day⁻¹ or more. Accordingly, with reference to this, the value of C can be assumed previously.

The cell density may also be replaced by the cell number or the cell aggregate size or volume. For example, in a case where the cell density is set to be the cell aggregate volume (C being the cell aggregate volume at an arbitrary time point during culture and C₀ being the cell aggregate volume at the start of the control), F (variable perfusion rate) when one of the culture variables is the cell aggregate volume increasing rate can be expressed by Formula 1 that is proportional to the cell aggregate volume increasing rate.

The following Formula 2 can be obtained by multiplying Formula 1 above by M as a correction coefficient for correcting the difference in cell characteristics or the like due to the cell line and the culture history of the cell line.

$$F = M \times {F}_{0} \times \frac{C}{{C}_{0}} \qquad [ Formula 2 ]$$

The difference in cell characteristics is not limited but includes resistance to lactic acid in the culture solution. It may be set to reflect the upper limit of the lactic acid concentration that does not have remarkable adverse effects on cells. The value of M may also be set by reflecting the lower limit in the above-described adjusted carbon dioxide gas concentration. In general, in a case where the lower limit for the adjusted carbon dioxide gas concentration is lower, the value of correction coefficient M may be lower. Although not particularly limited, for example, the correction coefficient M may be regarded as a value representing the difference in the resistance of the cell line to a stringent culture environment. As the absolute value of the correction coefficient M, the lower limit is preferably 0.1, 0.2, 0.3, 0.4, 0.5, 0.6, 0.7, 0.8, 0.9, or 1.0, and the upper limit is preferably 2.0, 1.9, 1.8, 1.7, 1.6, 1.5, 1.4, 1.3, 1.2, 1.1, or 1.0. The value of M may be positive or negative. For example, when the cell density, cell density increasing rate, cell number, or cell aggregate size or volume is used as culture variables, a positive M value is used because, generally, it is preferable to increase the amount of medium perfused as these variables increase. Meanwhile, for example, when pH is used instead of the cell density as a culture variable, a negative M value is used because, generally, it is preferable to increase the amount of medium perfused as the pH decreases.

As the value of the correction coefficient M, the value of the lactic acid resistance in the culture solution of the cell line used, with the lactic acid resistance in the culture solution of a certain human iPS cell line (e.g., cell line Ff-I14s04) set to 1.0, may be set. The lactic acid resistance of the cell line used may be determined based on, for example, an IC₅₀ value calculated from the culture with lactic acid being added or based on accumulated lactic acid concentrations before and after the cell growth began to decline in experimental culture. Information on lactic acid resistance may be provided by the provider of the cell line or obtained by actual measurement.

In addition, as the value of the correction coefficient M, a value that indicates resistance to low pH in the culture solution may also be used. In this case, for example, the value of M may also be set by reflecting the optimum pH of the cell line used or the lower limit of carbon dioxide gas concentration, which will be described later. In general, the higher the optimum pH and/or the lower the lower limit for the adjusted carbon dioxide gas concentration, the smaller the value of M may be. As the value of the correction coefficient M, the value of the pH resistance in the culture solution of the cell line used may be set, for example, with the pH resistance in the culture solution of a specific human iPS cell line set to 1.0. Information on pH resistance may be provided by the provider of the cell line or obtained by actual measurement.

It is also possible to multiply Formula 2 above by a variable K that varies depending on the amount of lactic acid produced by metabolism per cell per unit time to obtain the following Formula 3.

$$F = M \times K \times {F}_{0} \times \frac{C}{{C}_{0}} \qquad [ Formula 3 ]$$

K can be expressed by the following Formula 4 with the amount of lactic acid produced by metabolism per cell per unit time at a certain time point, L₀, and the amount of lactic acid produced by metabolism per cell per unit time in each subsequent culture time, L.

$$K = \frac{L}{{L}_{0}} \qquad [ Formula 4 ]$$

In this regard, the amount of lactic acid produced by metabolism per cell per unit time at a certain time point refers to a value obtained by dividing a change in the amount of lactic acid in the culture solution per unit time until the certain time point by the average cell number within the unit time (that is, a value obtained by dividing a change in the lactic acid concentration in the culture solution per unit time until the certain time point by the average cell density within the unit time). In this connection, as the amount of lactic acid or lactic acid concentration in the culture solution, for example, a value directly measured in the culture solution, a value measured in a sample collected in a small amount from the culture solution, or a value measured in the medium removed from the culture system by perfusion may be used.

The upper limit of the lactic acid concentration when the amount of medium perfused is started to be changed is preferably 10 mM, 9 mM, 8 mM, or 7 mM. When it is desirable to control the lactic acid concentration, if the lactic acid concentration when the amount of medium perfused is started to be changed is high, the amount of medium used for perfusion increases in general.

The amount of lactic acid produced by metabolism per cell per unit time may differ between cell lines or depending on culture conditions or the like. Therefore, it is preferable to measure and confirm the amount in advance in accordance with the cells to be used. In addition, it is preferable that the lower limit is 1.0×10⁻¹⁰ mmol/cell/h, 3.0×10⁻¹⁰ mmol/cell/h, 5.0×10⁻¹⁰ mmol/cell/h, 7.0×10⁻¹⁰ mmol/cell/h, 1.0×10⁻⁹ mmol/cell/h, 1.1×10⁻⁹ mmol/cell/h, 1.2×10⁻⁹ mmol/cell/h, or 1.3×10⁻⁹ mmol/cell/h, and that the upper limit is 2.5×10⁻⁹ mmol/cell/h, 2.0×10⁻⁹ mmol/cell/h, 1.9×10⁻⁹ mmol/cell/h, 1.8×10⁻⁹ mmol/cell/h, 1.7×10⁻⁹ mmol/cell/h, 1.6×10⁻⁹ mmol/cell/h, 1.5×10⁻⁹ mmol/cell/h, 1.4×10⁻⁹ mmol/cell/h, or 1.3×10⁻⁹ mmol/cell/h. Preferably, the lactic acid production rate in the suspension culture step is maintained between the lower and upper limits described above. In this regard, a change in the amount of lactic acid produced by metabolism per cell per unit time during culture can be also assumed on the basis of the HK2 gene expression level and change thereof.

It is preferable to control the amount of medium perfused during culture according to the above-described formulas in principle. However, applying the above-described formulas may be temporarily suspended, and the lactic acid concentration and pH in the culture solution may be restored to their assumed ranges by increasing or decreasing by arbitrary amount, or maintaining the amount of medium perfused in a case where the lactic acid concentration and pH in the culture solution and the like measured by arbitrary methods are deviated from the initially assumed ranges, namely the case where they are out of the ranges of values that do not adversely affect cells, or in a case where they are within the ranges of values that do not adversely affect cells but are continuously out of the initially assumed ranges, and perfusion with an extra amount of medium is required. The assumed ranges may be appropriately determined according to conditions such as the cost and equipment. Preferably, the assumed ranges are set within the ranges of the lactic acid concentration and pH that do not adversely affect cells. Examples of the upper limit of the lactic acid concentration that does not adversely affect cells include 20 mM, 18 mM, 16 mM, 14 mM, 13 mM, 12 mM, 11 mM, 10 mM, 9 mM, 8 mM, or 7 mM but are not particularly limited thereto because it may vary depending on the cell line or the like. Examples of the lower limit of pH that does not adversely affect cells include 6.5, 6.6, 6.7, 6.8, 6.9, 6.95, 7.0, 7.05, 7.10, and 7.14. Examples of the upper limit of pH that does not adversely affect cells include 9.0, 8.5, 8.0, 7.6, 7.5, 7.4, 7.3, 7.2, and 7.16. Preferably, the pH in the suspension culture step or at the start of control is maintained at the lower limit described above or more. Meanwhile, preferably, the lactic acid concentration in the culture solution in the suspension culture step or at the start of control is maintained at the upper limit described above or less.

After the cell density reached 8.0×10⁵ cells/mL, the degree of susceptibility to changes in the culture environment increases. Therefore, it is possible to control the amount of medium perfused such that the total amount of medium used for medium change for any 6 hours of culture after the cell density reached 8.0×10⁵ cells/mL becomes greater than the total amount of medium used for medium change for 6 hours of culture immediately before the any 6 hours of culture. In other words, the control of the amount of medium perfused can include increasing an amount of medium perfused for any 6 hours of culture after the cell density of the pluripotent stem cells reached 8.0×10⁵ cells/mL compared to an amount of medium perfused for 6 hours of culture immediately therebefore.

Performing a perfusion culture by the above-described method makes it possible to grow high-quality cells with high efficiency, and to produce a high-quality pluripotent stem cell stock.

The medium change by the perfusion mode may be performed by continuously removing the culture solution from which the cells have been separated by a filter or the like from the vessel while continuing the culture and continuously adding a new medium. The pore size of the filter to be used may be smaller than cell aggregates. In addition, the pore size may have a size through which dead cells or the like in the culture solution can pass. The lower limit is, but not particularly limited to, preferably 0.1 μm, 1 μm, 5 μm, 10 μm, or 20 μm, and the upper limit is, but is not particularly limited to, preferably 50 μm, 40 μm, 35 μm, 30 μm, 25 μm, 20 μm, or 15 μm.

In this suspension culture step, the cell number obtained through growth can be set arbitrarily. The cell number and state of interest may be appropriately determined according to the type of cells to be cultured, the purpose of cell aggregation, the type of medium, the culture conditions, and the number of cells desired for production of a stock. For example, the degree of cell growth in one passage period is not particularly limited, but the lower limit may be 2 times, 3 times, 5 times, 6 times, 7 times, 8 times, 8.5 times, 8.8 times, 8.9 times, 9 times, 9.1 times, 9.15 times, 10 times, 11 times, or 11.1 times compared to the cell seeding concentration at the start of culture. Meanwhile, the upper limit is not particularly set but may be, for example, 100 times, 50 times, 40 times, 30 times, 20 times, or 10 times. In particular, cells preferably grow 10 times or more. In addition, passages and cultures in a suspension culture may be repeated a plurality of times to grow the cells, for example, to 500 times or more, 1000 times or more, 1500 times or more, 2000 times or more, 2500 times or more, 15000 times or more, 150000 times or more, or 1500000 times or more the number of the raw material cells. The degree of cell growth may be measured, for example, on day 1 of culture, day 2 of culture, day 3 of culture, day 4 of culture, day 5 of culture, day 6 of culture, or later. In addition, measurements may be taken for a plurality of times on different days.

In the suspension culture step, it is possible to isolate a part of the pluripotent stem cells during culture and to confirm the cell number and the cell aggregate size. A cell aggregate of pluripotent stem cells taken out during culture may be loosened into single cells by, for example, enzymatic treatment, and the viable cell number may be measured by a method such as the trypan blue method. Alternatively, it is also possible to estimate the cell number from the number and size of cell aggregates of pluripotent stem cells taken out during culture. In addition, it is not particularly limited but the cell aggregate size or the cell aggregate volume can be measured by size measurement using a laser method, a method for acquiring an image and calculating the size from the image, or the like. In addition, the number of cells in a suspension culture can be calculated also by the dissolved oxygen concentration of the culture solution.

The size of a cell aggregate produced in this suspension culture step is not particularly limited, but the average diameter of the maximum width size in an observation image of cell aggregates in the same culture system when observed with a microscope may be the lower limit of 30 μm, 40 μm, 50 μm, 60 μm, 70 μm, 80 μm, 90 μm, or 100 μm, and the upper limit of 500 μm, 400 μm, 300 μm, 250 μm, 200 μm, or 150 μm. Cell aggregates within this range are preferable as a growth environment for cells because oxygen and nutrients are easily supplied to the cells inside. Particularly preferably, the cell aggregate size has a lower limit of 40 μm and an upper limit of 250 μm. In addition, the size of a cell aggregate formed by seeding cells in a suspension culture, for example, the size of a cell aggregate after 24 hours is preferably smaller, particularly preferably 100 μm or less, to enables high-quality, high-efficiency, and maximal growth in one passage period in the subsequent culture. In this regard, the sizes of all the cell aggregates in a culture solution do not need to be within the above-described range. For example, the average size by cell number may be within the above-described range.

In the cell aggregate population produced in this suspension culture step has a lower limit of 30%, 40%, 50%, 60%, 70%, 75%, 80%, 85%, 90%, 95%, 98%, or 100% of the cell aggregates on the weight basis is preferably within the above-described size.

In this suspension culture step, the medium removed from the culture system in the perfusion mode may be used for measuring the concentrations of nutrients and metabolites in the medium. For example, although not limited, it is possible to measure the glucose concentration, lactic acid concentration, or the like in the removed medium using a medium component measuring device using an enzymatic electrode reaction. The information above may be reflected in the control of amount of medium perfused.

The glucose concentration in the medium removed from the culture system by the perfusion mode preferably has a lower limit of 1 mM, 2 mM, 3 mM, 4 mM, 5 mM, 6 mM, 7 mM, 8 mM, 9 mM, or 10 mM and an upper limit of 20 mM, 19 mM, 18 mM, 17 mM, 16 mM, 15 mM, 14 mM, 13 mM, 12 mM, or 11 mM, but are not particularly limited. For example, the lower limit may be 4 mM, and the upper limit may be 16 mM. In addition, the lactic acid concentration in the medium removed from the culture system by the perfusion mode preferably has a lower limit of 0 mM, 1 mM, 2 mM, 3 mM, 4 mM, 5 mM, 6 mM, 7 mM, 8 mM, 9 mM, or 10 mM and an upper limit of 20 mM, 19 mM, 18 mM, 17 mM, 16 mM, 15 mM, 14 mM, 13 mM, 12 mM, 11 mM, 10 mM, 9 mM, 8 mM, 7 mM, or 6 mM. For example, the lower limit may be 0 mM, and the upper limit may be 12 mM.

In the suspension culture step in this perfusion mode, it is possible to isolate a part of the pluripotent stem cells during culture and to confirm the cell number or whether the cells maintain the undifferentiated state. For example, it is possible to confirm whether the undifferentiated state is maintained by measuring the expression of pluripotent stem cell markers expressed in pluripotent stem cells isolated during culture. For example, a pluripotent stem cell marker may include Alkaline Phosphatase, Nanog, OCT4, SOX2, TRA-1-60, c-Myc, KLF4, LIN28, SSEA-4, and SSEA-1. Examples of the method for detecting these pluripotent stem cell markers include flow cytometry, as described above.

When the positive rate for pluripotent stem cell markers in pluripotent stem cells isolated during culture is, for example, 80% or more, 90% or more, 91% or more, 92% or more, 93% or more, 94% or more, 95% or more, 96% or more, 97% or more, 98% or more, 99% or more, or 100% or less, it can be determined that the undifferentiated state is maintained. When a plurality of pluripotent stem cell markers are used, the positive rate and the undifferentiated property are determined as described above.

In addition, in this step, it is possible to confirm whether the undifferentiated state is maintained by measuring the expression of three germ layer markers (endodermal cell marker, mesodermal cell marker, and ectodermal cell marker) in the pluripotent stem cells isolated during culture. In other words, when the positive rate for all of these endodermal cell marker, mesodermal cell marker, and ectodermal cell marker are, for example, 20% or less, 10% or less, 9% or less, 8% or less, 7% or less, 6% or less, 5% or less, 4% or less, 3% or less, 2% or less, 1% or less, or the detection limit or less, it can be determined that the undifferentiated state is maintained. Alternatively, when the degree of expression is a given or lower degree, compared with the expression level of each marker in a cell population after induction of differentiation, the undifferentiated state can be judged to be maintained. Specifically, for example, when the degree of expression is a 10th or less, 50th or less, 100th or less, 200th or less, 300th or less, 400th or less, 500th or less, or 600th or less of the expression level in a cell population after induction of differentiation, it can be determined that the undifferentiated state is maintained.

An “endodermal cell marker” refers to a gene specific to endodermal cells. Examples thereof may include SOX17, FOXA2, CXCR4, AFP, GATA4, and EOMES. An endodermal cell forms tissues of organs such as the digestive tract, lung, thyroid, pancreas, and liver, cells of secretory glands opening to the digestive tract, peritoneum, pleura, larynx, auditory tube, trachea, bronchi, urinary tracts (bladder most of the urethra, part of urinary duct), and the like.

A “mesodermal cell marker” refers to a gene specific to mesodermal cells. Examples thereof may include T (BRACHYURY), MESP1, MESP2, FOXF1, HAND1, EVX1, IRX3, CDX2, TBX6, MIXL1, ISL1, SNAI2, FOXC1, and PDGFRα. A mesodermal cell forms body cavities and lining mesothelium, muscle, skeleton, skin dermis, and connective tissue, heart, blood vessels (including vascular endothelium), blood (including blood cells), lymph vessels, spleen, kidneys, ureters, gonads (testis, uterus, gonadal epithelium), and the like.

An “ectodermal cell marker” refers to a gene specific to ectodermal cells. Examples thereof may include FGF5, NESTIN, SOX1, and PAX6. An ectodermal cell forms the epidermis of the skin and epithelium of the terminal urethra in males, hair, nails, skin glands (including mammary and sweat glands), sensory organs (including the terminal epithelium of the oral cavity, pharynx, nose, and rectum, salivary glands), lens, peripheral nervous system, and the like. In addition, part of the ectoderm is invaginated into grooves during development to form the neural tube and also serves as the origin of neurons and melanocytes in the central nervous system, such as the brain and spinal cord.

The expression of these three germ layer markers (endodermal cell marker, mesodermal cell marker, and ectodermal cell marker) can be measured by any detection method in the art. Examples of the method for measuring the expression of three germ layer markers (endodermal cell marker, mesodermal cell marker, and ectodermal cell marker) include, but are not limited to, quantitative real-time PCR analysis, the RNA-Seq method, northern hybridization, or hybridization methods using DNA array, as well as the methods using flow cytometry described for the pluripotent stem cell markers. In quantitative real-time PCR analysis, the expression level of the marker to be measured is calculated into the relative expression level with respect to the expression level of an internal standard gene, and the expression level of the marker can be evaluated based on the relative expression level. Examples of an internal standard gene include the glyceraldehyde-3-phosphate dehydrogenase (GAPDH) gene and the β-actin (ACTB or bAct) gene. This detection method may also be used for analyzing the expression of pluripotent stem cell markers described above.

The lower limit of the specific growth rate of cells upon termination of this step is preferably 0.2 day⁻¹, 0.3 day⁻¹, 0.4 day⁻¹, 0.5 day⁻¹, or 0.6 day⁻¹. In addition, the upper limit of the specific growth rate is not particularly limited. For example, it is preferably 1.5 day⁻¹, 1.4 day⁻¹, or 1.3 day⁻¹. The specific growth rate refers to a cell-increasing rate per unit time, and herein refers to a cell-increasing rate per day (24 hours) in particular. For cells in culture, the specific growth rate at a time point refers to a cell-increasing rate per 24 hours immediately before the time point.

### (Collection of Cell in Suspension Culture)

A cell after suspension culture is collected to be used for the subsequent stock preparing step. In this regard, this collection operation is the same as the operation during a passage from suspension culture to suspension culture in the suspension culture step. In the step of collecting the pluripotent stem cell subjected to suspension culture, the pluripotent stem cell is separated from the culture solution by a conventional method, and the pluripotent stem cell separated is collected. In this connection, the pluripotent stem cell is preferably collected as a single-state cell by dispersion treatment from an adjacent pluripotent stem cell. That is, this step preferably includes a step of unicellularizing a cell aggregate. The single-state cell may be in a state in which a single cell (a cell in a unicellular state) dispersed from a cell aggregate exists. All the cells are not necessarily in a single free state, but a plurality of cells in an adherent state may exist.

After the suspension culture step, the cells or pluripotent stem cell population exist in a suspended state in the culture solution. Therefore, the collection thereof may be achieved by removing the liquid component of the supernatant by standing or centrifugation. They may also be collected using a filtration filter, a hollow fiber separation membrane, or the like. In the case of removing the liquid component by standing, the vessel containing the culture solution is allowed to stand for about 5 minutes, and the supernatant may be removed while leaving the sedimented cells or pluripotent stem cell population such as cell aggregates. In the case of removing the liquid component by centrifugation, the centrifugal acceleration and the treatment time may be such that the centrifugal force does not damage the cells. For example, the lower limit of the centrifugal acceleration is not particularly limited as long as cells can be sedimented, and may be, for example, 50×g, 100×g, 200×g, 300×g, 800×g, or 1000×g. Meanwhile, the upper limit may be any speed at which the cells are not or not easily damaged by the centrifugal force, and may be, for example, 1200×g, 1500×g, or 2000×g. The lower limit of the treatment time is not particularly limited, as long as it is the time by which the above-described centrifugal acceleration can sediment cells, and may be, for example, 30 seconds, 1 minute, 3 minutes, or 5 minutes. The upper limit thereof may be any time by which the cells are not or not easily damaged by the above-described centrifugal acceleration, and may be, for example, 20 minutes, 10 minutes, 8 minutes, 6 minutes, or 5 minutes. In the case of removing the liquid component by filtration for collecting cell aggregates, for example, the culture solution may be passed through a nonwoven fabric or a mesh filter for removing the filtrate, thereby collecting the remaining cell aggregates. In the case of removing the liquid component by a hollow fiber separation membrane, for example, the culture solution and cells may be separated and collected using an apparatus equipped with a hollow fiber separation membrane, such as a cell concentration and washing system (KANEKA CORPORATION).

The collected cells may be washed, if necessary. The washing method is not limited. Buffer (such as PBS buffer), physiological saline, or a medium (preferably basal medium) may be used as a washing solution.

An enzymatic detachment agent and/or a chelating agent can be used for unicellularization. The enzymatic detachment agent is not particularly limited. It is possible to use not only a commercially available detachment agent but also an enzyme that weakens the intercellular junction in the cell aggregate, thus enabling the cells to be unicellularized. Examples of the enzymatic detachment agent include trypsin, collagenase, pronase, hyaluronidase, elastase, and commercially available products such as Accutase (registered trademark), Accumax (registered trademark), TrypLE™ Express Enzyme (Life Technologies Japan Ltd.), TrypLE™ Select Enzyme (Life Technologies Japan Ltd.), and Dispase (registered trademark). Examples of a chelating agent that may be used include, but are not particularly limited to, EDTA and EGTA. For example, in the case of using trypsin for unicellularization, the lower limit of the concentration in the solution is not particularly limited as long as the pluripotent stem cell population can be dispersed, and may be, for example, 0.15% by volume, 0.18% by volume, 0.20% by volume, or 0.24% by volume. Meanwhile, the upper limit of the concentration in the solution is not particularly limited as long as the concentration does not cause an effect such as cells per se to be dissolved, and may be, for example, 0.30% by volume, 0.28% by volume, or 0.25% by volume. The treatment time depends on the concentration of trypsin, but the lower limit thereof is not particularly limited as long as the pluripotent stem cell population is well dispersed by the action of trypsin within the time. It may be, for example, 2 minutes, 3 minutes, 5 minutes, 8 minutes, 10 minutes, 12 minutes, or 15 minutes. Meanwhile, the upper limit of treatment time is not particularly limited as long as it does not cause an effect such as cells per se to be dissolved by the action of trypsin, and may be, for example, 30 minutes, 28 minutes, 25 minutes, 22 minutes, 20 minutes, or 18 minutes. When a commercially available enzymatic detachment agent is used, it may be used at a concentration that allows the cells to be dispersed in a single-cell state, as described in the attached protocol.

For example, when EDTA is used for unicellularization, the lower limit of the concentration in the solution is not particularly limited as long as the pluripotent stem cell population can be dispersed, and is preferably, for example, 0.01 mM, 0.1 mM, or 0.5 mM. Meanwhile, the upper limit of the concentration in the solution is not particularly limited as long as the concentration does not cause an effect such as cells per se to be dissolved, and is preferably 100 mM, 50 mM, 10 mM, or 5 mM. It is preferable to use one or more types of both enzymatic detachment agent and chelating agent for unicellularization. In addition, it is preferable in the unicellularization that the enzymatic detachment agent and the chelating agent which treat a cell contain no ROCK inhibitor. The presence of a ROCK inhibitor firms the junction between cells in a pluripotent stem cell population such as a cell aggregate, thus making it difficult for the population to be unicellularized. After treatment with the enzymatic detachment agent and/or chelating agent, unicellularization may be promoted by applying mild force to a pluripotent stem cell population such as the cell aggregate treated. This stress-applying treatment is not particularly limited. Conceivable examples include physical stimuli such as: a method in which cells together with the solution are pipetted a plurality of times; a method in which a Taylor vortex flow is generated to apply a shear stress; stirring with a stirring blade; or the like. Further, the cells may be passed through a strainer or mesh, if necessary. In this regard, the unicellularization treatment with an enzymatic detachment agent is herein referred to as an enzymatic treatment.

The unicellularized cell may be collected by removing the supernatant containing the detachment agent via standing, centrifugation, or the like. The cell collected may be suspended directly or, if necessary, with a buffer (such as a PBS buffer), physiological saline, a cell storage solution used in the cell stock preparing step, or a medium (preferably containing a ROCK inhibitor), and then subjected to the subsequent step.

In addition, the cell survival rate upon termination of this step is preferably, for example, 92% or more, 93% or more, 94% or more, 95% or more, 96% or more, 97% or more, 98% or more, or 99% or more.

In this step, a passage can be performed. In this case, the number of passages is not particularly limited. For example, the number of passages can be 0, 1 or more, 2 or more, 3 or more, or 4 or more. The upper limit is not particularly limited. The passaging method is not particularly limited. A passage can be performed, for example, by collecting cell populations using the method above-described for a medium change, and again seeding the cells unicellularized by the above-described method. The absolute number of cells to be finally obtained can be increased to any desired number, as follows: one or more passages are performed in this step; and the passage is performed in a larger vessel than before the passage, the amount of medium to be used is increased, or the passage is performed in a plurality of separate vessels. In this regard, the time to be taken until the cells are used in the passage culture is not particularly limited. From the viewpoint of maintaining the quality of the cells, the subsequent passage step is preferably performed promptly. The standby time (the time from the completion of collection to the start of seeding in a passage) is, for example, 24 hours or less, 18 hours or less, 12 hours or less, 10 hours or less, 8 hours or less, 6 hours or less, 4 hours or less, 3 hours or less, 2 hours or less, or 1 hour or less. In a case where the standby time is long in relation to the schedule of the steps, the cells in a unicellularized state, which are suspended in a liquid with which the cells have been washed, are desirably stored at low temperature (e.g., 10° C. or less or 5° C. or less). In this regard, in this standby period, the cells are preferably not freeze-preserved.

In the present invention, performing an adherent culture step and a suspension culture step under the above-described preferable conditions can bring the number of cells at the end of culture in the suspension culture step to 1×10⁸ cells or more, or even, depending on the condition, 5×10⁸ cells or more, 1×10⁹ cells or more, or further, 2×10⁹ cells or more, even in a case where the number of raw material cells used in the adherent culture is, for example, 1×10⁶ cells or less.

### 1-3-3. Cell Stock Preparing Step

The “cell stock preparing step” is a step in which cells cultured and collected in a suspension culture step are suspended in a storage solution, and aliquoted in desired cell count into each desired vessel to prepare cell stocks. For preparation of a cell stock, a cell storage method known in the art may be used. For example, a method in which cells are suspended in a freeze-preservation solution, and the resulting suspension is aliquoted into freezing vials, and slowly frozen may be used.

### (Cells)

Cells used in this step are cells cultured and collected in “1-3-2. Suspension Culture Step” above. The pluripotent stem cells used in this step may be a cell population (pluripotent stem cell population) composed of a plurality of cells, and expresses a pluripotent stem cell marker (e.g., OCT4, SOX2, NANOG, SSEA-4, or TRA-1-60) in the pluripotent stem cell population. For pluripotent stem cells used to prepare a stock in this step, it is preferable that a proportion of the cells positive for OCT4 is 90% or more, and that a proportion of the cells positive for TRA-1-60 is 90% or more. It is more preferable that the proportion of the cells positive for OCT4 is 91% or more, 92% or more, 93% or more, 94% or more, 95% or more, 96% or more, 97% or more, 98% or more, 99% or more, or 100%, and that the proportion of the cells positive for TRA-1-60 is 91% or more, 92% or more, 93% or more, 94% or more, 95% or more, 96% or more, 97% or more, 98% or more, 99% or more, or 100%. In addition, for the cells used in this step, it is preferable that the specific growth rate in a suspension culture per (last) 24 hours immediately before is 0.60 day⁻¹ or more, 0.65 day⁻¹ or more, 0.70 day⁻¹ or more, 0.75 day 1 or more, 0.80 day⁻¹ or more, 0.85 day⁻¹ or more, 0.86 day⁻¹ or more, 0.87 day⁻¹ or more, 0.88 day⁻¹ or more, 0.89 day⁻¹ or more, 0.90 day⁻¹ or more, or 0.91 day⁻¹ or more. Such a cell achieves a high survival rate, and in addition, can produce a high-quality cell stock that excels in the adhesion rate, aggregate-forming capability, start-up of growth, and the like when used for culture.

In addition, the cell survival rate before preparation of the cells as a stock is preferably, for example, 92% or more, 93% or more, 94% or more, 95% or more, 96% or more, 97% or more, 98% or more, or 99% or more.

Further, the adhesion rate of the cells that have been prepared and stored as a stock, and then seeded in an adherent culture is preferably, for example, 40% or more, 45% or more, 50% or more, 60% or more, 70% or more, 80% or more, 90% or more, 95% or more, or 100%. The adhesion rate is usually calculated as a proportion of the number of cells adhering at a predetermined time point after the start of culture with respect to the number of the cells seeded. In this case, the predetermined time point is not particularly limited, and for example, the adhesion rate can be calculated at the time point 24 hours after the start of culture. In some of such cases, the cells are grown already during the time from the start of culture, and the adhesion rate exceeds 100%. In some cases, the adhesion rate of those cells of a stock according to the present invention which are seeded after being stored is, for example, 100% or more, 105% or more, 106% or more, 107% or more, 110% or more, 115% or more, 116% or more, or 117% or more.

In addition, the aggregate-forming rate of the cells prepared and stored as a stock, and then seeded in suspension culture is preferably, for example, 80% or more, 85% or more, or 90% or more. The aggregate-forming rate is usually calculated as a proportion, with respect to the number of cells seeded, of the number of cells in the form of cell aggregates at a predetermined time point after the start of culture. In this case, the predetermined time point is not particularly limited, and for example, the aggregate-forming rate can be calculated at the time point 24 hours after the start of culture. In some of such cases, the cells are grown already during the time from the start of culture, and the aggregate-forming rate exceeds 100%. In some cases, the aggregate-forming rate of those cells of a stock according to the present invention which are seeded after being stored is, for example, 100% or more, 105% or more, 110% or more, 111% or more, 112% or more, 115% or more, 120% or more, 123% or more, 125% or more, or 127% or more.

In addition, the cell survival rate of the cells prepared and stored as a stock is preferably, for example, 92% or more, 93% or more, 94% or more, 95% or more, or 96% or more.

In the cells prepared and stored as a stock, a proportion of the cells in the G0/G1 phase in the cell cycle is preferably 26% or less, 25% or less, 20% or less, 18% or less, 16% or less, or 15% or less. Alternatively, a proportion of the cells in the G2/M phase with respect to the cells in the G0/G1 phase is preferably 1.1 times or more, 1.2 times or more, 1.3 times or more, 1.4 times or more, 1.5 times or more, 1.6 times or more, 1.7 times or more, 1.8 times or more, 1.9 times or more, 2.0 times or more, 2.1 times or more, 2.2 times or more, 2.3 times or more, 2.4 times or more, 2.5 times or more, 2.6 times or more, or 2.65 times or more. Alternatively, a proportion of the cells in the G0/G1 phase with respect to the cells in the S phase is preferably 0.65 times or less, 0.6 times or less, 0.5 times or less, 0.4 times or less, 0.39 times or less, 0.38 times or less, or 0.37 times or less.

### (Vessel)

A vessel in which a cell stock prepared is contained and stored is not particularly limited. A vessel the inner face of which is treated to suppress adsorption of protein is preferable. The type of vessel which can be sealed is preferable. A vessel storable in liquid nitrogen is preferable. The usable form is, for example, a vial type, bag type, tube type, or the like. A commercially available storage vessel can be used. Examples of a commercially available vessel that can be used include Nunc Cryo Tube (Thermo Fisher Scientific Inc.), Nalgene Cryo Vial (Thermo Fisher Scientific Inc.), Bi. File Jacket Tube (FCR & Bio Co., Ltd.), and the like. The capacity of the vessel is not particularly limited as long as the vessel can be filled with a sufficient amount of a storage solution having cells suspended therein. For example, the lower limit may be 0.1 mL, 0.5 mL, or 1.0 mL, and the upper limit may be 1000 mL, 500 mL, 100 mL, 50 mL, 10 mL, or 5 mL.

### (Storage Solution)

As a storage solution used to prepare a cell stock in which cells acquired in the suspension culture step are suspended, any freeze-preservation solution, cold-storage solution, buffer solution, or the like, as described in the section “Cell Stock” in “1-2. Definitions of Terms” above, can be used. A freeze-preservation solution is particularly preferable. In addition, the storage solution may contain a ROCK inhibitor. In addition, the density of the cells suspended in a storage solution may be not a density that particularly decreases a quality such as the survival rate of the cells. For example, the lower limit is preferably 0.1×10⁶ cells/mL, 0.2×10⁶ cells/mL, 0.3×10⁶ cells/mL, 0.4×10⁶ cells/mL, 0.5×10⁶ cells/mL, 0.6×10⁶ cells/mL, 0.7×10⁶ cells/mL, 0.8×10⁶ cells/mL, 0.9×10⁶ cells/mL, or 1.0×10⁶ cells/mL. The upper limit is preferably 100×10⁶ cells/mL, 50×10⁶ cells/mL, 10×10⁶ cells/mL, 9×10⁶ cells/mL, 8×10⁶ cells/mL, 7×10⁶ cells/mL, 6×10⁶ cells/mL, 5×10⁶ cells/mL, 4×10⁶ cells/mL, 3×10⁶ cells/mL, or 2×10⁶ cells/mL.

A temperature at which cells are suspended in a storage solution and filled into a vessel to prepare a cell stock is preferably lower. A storage solution often contains a component toxic to a cell, and in addition, a cell in a unicellular state is unstable and prone to cause cell death or the like. Hence, preparing a cell stock while suppressing toxicity and the excess activity of a cell at low temperature makes it possible to suppress a decrease in a quality such as the survival rate of a cell. In this case, the low temperature may be a temperature at which the cell suspension is not frozen. For example, the lower limit is 0° C., 1° C., or 2° C. The upper limit is not particularly limited, and is preferably, for example, 12° C., 10° C., 9° C., 8° C., 7° C., 6° C., 5° C., or 4° C. for the above-described reason. In the present invention, such preferable filling conditions can be achieved by performing this filling step in a state where the vessel or the cell suspension is retained on a low-temperature base material of 10° C. or less, or performing the filling step under a low-temperature environment of 10° C. or less.

### (Filling)

A method for filling a storage vessel with a storage solution having cells suspended therein is not particularly limited. For example, the vessel may be filled using a micropipette, may be filled using an autopipetter, may be filled using a syringe, may be filled using a multi-channel micropipette, or may be filled using an automatic aliquoting device. A particularly preferable method is use of a multi-channel micropipette or an automatic aliquoting device that can each efficiently fill a large number of storage vessels. A still more preferable method is a method in which a multi-channel micropipette is used to fill a plurality of vessels simultaneously.

The time required for filling is preferably shorter in order to quickly achieve a state that allows storage so as not to decrease the quality of cells. Without particular limitation, the upper limit is preferably, for example, 150 minutes, 120 minutes, 90 minutes, 60 minutes, 50 minutes, 40 minutes, 30 minutes, 20 minutes, 10 minutes, or 5 minutes. In such a time required, filling at low temperature in the same manner as suspending cells in a storage solution at low temperature as above-described makes it possible to minimum a decrease in the quality of a cell stock. In this case, the low temperature may be a temperature at which the cell suspension is not frozen. For example, the lower limit is 0° C., 1° C., or 2° C. The upper limit is not particularly limited, and is preferably, for example, 12° C., 10° C., 9° C., 8° C., 7° C., 6° C., 5° C., or 4° C. for the above-described reason. Such a preferable filling temperature can be achieved in a state where the vessel or the cell suspension is retained on a low-temperature base material of 10° C. or less, or can be achieved under a low-temperature environment of 10° C. or less. Accordingly, the cell suspension may be allowed to stand by in the above-described low-temperature environment for the above-described time, for example, 180 minutes, 150 minutes, 120 minutes or less, 90 minutes or less, 60 minutes or less, 50 minutes or less, 40 minutes or less, 30 minutes or less, 20 minutes or less, 10 minutes or less, or 5 minutes or less, from the termination of collection of the cells to the storage (to the start of freezing in a case where the suspension is to be frozen), regardless of whether the time is required for the filling or not.

In addition, the number of storage vessels to be filled, that is, the number of vessels for stock storage into which cells are aliquoted is a desired number that may be set appropriately, considering the number of cells collected in a suspension culture, the amount of the solution for the filling, the cell density in a storage solution, and the like. However, considering the nature of being a cell stock, the lower limit of the number is, for example, but not particularly limited to, 20, 50 or more, 100 or more, 200 or more, or 300 or more. Meanwhile, in accordance with the form of utilization of a cell stock, for example, one to several cell stocks containing 1×10⁹ cells or more per vessel are prepared in some cases.

### (Storage)

A cell stock may be produced by Storing a cell stock solution (a storage solution having cells suspended therein) prepared after being filled into a storage vessel. The storage may be, for example, maintaining in a frozen state, may be maintaining in a cold-storage state, or may be maintaining in a gel state. Particularly preferably, the maintenance is in a frozen state. Bringing about a frozen state requires freezing a cell stock solution. The freezing method is, for example, a slow freezing method or a rapid cooling method, more preferably a slow cooling method. A slow cooling method is a freezing method with the temperature decreasing gradually. The upper limit of the cooling rate is preferably, for example, 3° C./min, 2.5° C./min, or 2° C./min, and the lower limit is preferably 0.5° C./min, 1.0° C./min, 1.5° C./min, or 2° C./min. In addition, at temperatures in the largest-crystal-nucleation temperature range, the cooling rate may be temporarily rendered higher than the above-described range to prevent both thawing due to heat generation and the subsequent refreezing. In addition, when the temperature is passed through the largest-crystal-nucleation temperature range and the solution is in a completely frozen state, the subsequent cooling rate may be out of the above-described range. For example, the cell stock solution may be frozen down to −80° C. by a slow cooling method, and then, immediately transferred to a storage vessel in liquid nitrogen to be cooled rapidly.

In a method for producing a pluripotent stem cell stock according to the present invention, a series of steps, namely, (a) a thawing step, (b) an adherent culture step, (c) a suspension culture step, (d) a step of aliquoting into a storage vessel, and (e) a cell freezing step, is preferably continuously performed. Performing these steps continuously, and shifting from one step to another promptly enables production of a cell stock having higher quality. Here, performing continuously does not necessarily mean performing all the steps in the same facilities. However, to rapidly perform the transition between the steps, it is preferable to perform in the same facilities, or perform in the facilities adjacent to each other or close to each other in distance. The time between one step and another is not particularly limited, and may be, for example, the time above-described as an example of, the standby time until use in a passage culture or suspension culture step, the time from the termination of collection of cells to the storage, or the time required for the filling.

In a method for producing a pluripotent stem cell stock according to the present invention, the production time in a series of steps, namely, (a) a thawing step, (b) an adherent culture step, (c) a suspension culture step, (d) a step of aliquoting into a storage vessel, and (e) a cell freezing step, depends on the culture period in a culture step or on whether to perform passage, is not particularly limited, and is, for example, 5 days or more, 7 days or more, 10 days or more, or 12 days or more, and 3 months or less or 2 months or less.

### 1-4. Effects

According to a method for producing a pluripotent stem cell stock according to the present invention, first performing an adherent culture on raw material cells having unstable quality can improve the quality of the resulting cell stock. In particular, differently from a conventional technology according to which a smaller number of passages is better, a passage is preferably repeated to perform adherent culture for two passage periods, thereby further stabilizing and recovering the quality. Then, performing suspension culture to grow a large number of cells to cells in numbers required to prepare a cell stock enables production of a large number of cell stocks from a small number of raw material cells. As a result, the number of cells at the end of the suspension culture can be, for example, 1×10⁸ cells or more, 2×10⁸ cells or more, 5×10⁸ cells or more, 1×10⁹ cells or more, or even, depending on the condition, 5×10⁹ cells or more, or 1×10¹⁰ cells or more. In addition, as cell stocks containing 1×10⁷ cells or more per 1 vessel, for example, 20 stocks or more, 30 stocks or more, 50 stocks or more, or 100 stocks or more can be produced, or as cell stocks containing 1×10⁹ cells or more per 1 vessel, 1 stock or more, 2 stocks or more, or 10 stocks or more can be produced. In addition, high-quality cells can be more efficiently obtained preferably by adjusting the carbon dioxide gas concentration or perfusing the medium so that the environment of a culture solution during a culture in the suspension culture step can be suitable for cells. In addition, when a cell stock is prepared, preferably performing the preparation work at low temperature can minimize a decrease in the quality of the cell stock. That is, the method according to the present invention can grow a large number of rare clinical raw material cells simply and efficiently, and further, can produce a high-quality cell stock, although such growth is difficult by a method performed using only adherent culture or only suspension culture.

### 2. Method for Improving Quality of Pluripotent Stem Cell

The present invention is also a method for improving the quality of pluripotent stem cells, including a step of adherent culture of freeze-preserved pluripotent stem cells after being thawed, and a step of suspension culture of the cells subjected to the adherent culture. Here, examples of the quality of a pluripotent stem cell include, but are not particularly limited to, the survival rate of a cell population and/or the adhesion rate to a culture base material. As preferable conditions for the adherent culture step, suspension culture step, and other steps, the conditions above-described in “1. Method for Producing Pluripotent Stem Cell Stock” can be adopted. In addition, the adhesion rate of the pluripotent stem cells used for adherent culture is preferably 70% or less. Further, a clinical cell line is preferably used as a pluripotent stem cell.

### 3. Pluripotent Stem Cell Stock

A pluripotent stem cell stock obtained by a preferable method for producing a pluripotent stem cell stock according to the present invention has characteristics that are not possessed by a conventional stem cell stock, and is very excellent from a quality viewpoint. That is, a novel pluripotent stem cell stock produced by the method described in the section “1. Method for Producing Pluripotent Stem Cell Stock” is also one aspect of the present invention.

For example, a storage solution to be used for production of, the number of cells to be contained in, and the form of storage of, a pluripotent stem cell stock according to the present invention may be as described in the section “1. Method for Producing Pluripotent Stem Cell Stock”, and among these, conditions regarded as preferable may be adopted. A pluripotent stem cell stock according to the present invention has a noticeably high quality in terms of the survival rate and the utilization efficiency, for example, compared with a cell stock generally produced by a known conventional method such as an adherent culture method.

The survival rate of a pluripotent stem cell stock according to the present invention is preferably 80% or more, 85% or more, 90% or more, 93% or more, 95% or more, or 96% or more when the cells are thawed. In addition, when a pluripotent stem cell stock after thawing is subjected to adherent culture, a proportion (the cell adhesion rate) of the number of viable cells adhering after 24 hours from seeding with respect to the number of cells seeded is preferably 80% or more, 90% or more, 100% or more, 105% or more, 106% or more, 107% or more, 110% or more, 115% or more, 116% or more, or 117% or more. A cell stock that exhibits such a high adhesion rate of cells is expected, for example, to reduce the number of days of culture, and improve the efficiency of induction of differentiation, and thus, can be said to have very high quality.

In addition, when a stock after thawing is subjected to suspension culture, a proportion (the aggregate-forming rate) of the number of viable cells forming cell aggregates after 24 hours from seeding with respected to the number of cells seeded is preferably 80% or more, 90% or more, 100% or more, 105% or more, 110% or more, 111% or more, 112% or more, 115% or more, 120% or more, or 123% or more, or 125% or more, or 127% or more. A cell stock that exhibits such a high aggregate-forming rate is expected to reduce the number of days of culture, and improve the efficiency of induction of differentiation, and thus, can be said to have very high quality.

The cell survival rate after thawing of a stock is preferably, for example, 92% or more, 93% or more, 94% or more, 95% or more, or 96% or more.

Further, the cell cycle of the cells contained in a pluripotent stem cell stock according to the present invention was examined. As a result, a proportion of the cells in the G2/M phase with respect to the cells in the G0/G1 phase has been revealed to be very high, compared with a conventional cell stock. The cell cycle is closely related to the proliferativeness, survival rate, and differentiation-inducing capability of a cell. Hence, a conceivable possibility is that, because a pluripotent stem cell stock of a cell population according to the present invention has the above-described cell-cycle characteristics, the cells have high efficiency and high quality.

In the cell cycle of a pluripotent stem cell stock according to the present invention, a proportion of the cells in the G0/G1 phase is preferably 26% or less, 25% or less, 20% or less, 18% or less, 16% or less, or 15% or less.

In the cell cycle of a pluripotent stem cell stock according to the present invention, a proportion of the cells in the G2/M phase with respect to the cells in the G0/G1 phase is preferably 1.1 times or more, 1.2 times or more, 1.3 times or more, 1.4 times or more, 1.5 times or more, 1.6 times or more, 1.7 times or more, 1.8 times or more, 1.9 times or more, 2.0 times or more, 2.1 times or more, 2.2 times or more, 2.3 times or more, 2.4 times or more, 2.5 times or more, 2.6 times or more, or 2.65 times or more.

In the cell cycle of a pluripotent stem cell stock according to the present invention, a proportion of the cells in the G0/G1 phase with respect to the cells in the S phase is preferably 0.65 times or less, 0.6 times or less, 0.5 times or less, 0.4 times or less, 0.39 times or less, 0.38 times or less, or 0.37 times or less.

### EXAMPLES

Hereinafter, the method for producing a pluripotent stem cell population according to the present invention will be described in more detail with reference to Examples. However, the scope of the present invention is not intended to be limited by the following Examples.

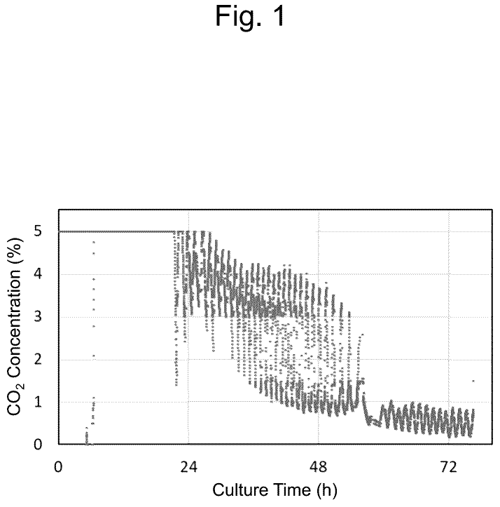

Production Examples 1 to 7, Comparative Examples 1, Examples 1 to 5, and Evaluation Examples 1 to 10 in the present application correspond to the Production Examples, Comparative Examples, Examples, and Evaluation Examples in Japanese Patent Application No. 2021-206065, on which the priority of the present application is based. Tables 1 to 11 and FIGS. 1 to 6 in the present application correspond to the Tables 1 to 11 and FIGS. 1 to 6 in Japanese Patent Application No. 2021-206065, on which the priority of the present application is based.

### Production Example 1: Adherent Culture of Human iPS Cell Line Ff-I14s04

Human iPS cells of the Ff-I14s04 cell line (The Center for iPS Cell Research and Application, Kyoto University), in a frozen state, were thawed, then seeded at 6000 cells/cm² in a 25 cm² culture flask coated with iMatrix-511 MG (Matrixome, Inc.) at 0.5 μg/cm², and subjected to adherent culture at 37° C. in a 5% CO₂ atmosphere. StemFit (registered trademark) AK03N (AJINOMOTO CO., INC.) was used as a medium. Given that the day on which the cells were seeded was day 0 of culture, the whole amount of the medium was changed on day 1 of culture, day 3 of culture, and day 5 of culture. The amount of the medium was 5 mL. Y-27632 (FUJIFILM Wako Pure Chemical Corporation) was added to the medium at a final concentration of 10 μM only at the time of cell seeding. On day 6 of culture, the cells were treated for a passage for 15 minutes with TrypLE™ Select Enzyme (Life Technologies Japan Ltd.) supplemented with 10 μM of Y-27632 (FUJIFILM Wako Pure Chemical Corporation), and the cells were detached from the culture surface, and being dispersed as single cells by pipetting. The cells were suspended and collected in StemFit (registered trademark) AK03N (AJINOMOTO CO., INC.) containing Y-27632 at a final concentration of 10 μM.

### Production Example 2: Adherent Culture of Human iPS Cell Line Ff-I14s04

The cells cultured and collected in Production Example 1 were seeded at 4000 cells/cm² in a 300 cm² culture flask coated with Vitronectin (VTN-N) Recombinant Human Protein, Truncated (Thermo Fisher Scientific Inc.) at 0.5 μg/cm², and subjected to adherent culture at 37° C. in a 5% CO₂ atmosphere. StemFit (registered trademark) AK03N (AJINOMOTO CO., INC.) was used as a medium, and the whole amount of the medium was changed on day 2 of culture and day 3 of culture. The amount of medium was 60 mL from day 0 of culture to day 3 of culture, and 90 mL from day 3 of culture to day 4 of culture. Y-27632 (FUJIFILM Wako Pure Chemical Corporation) at a final concentration of 10 μM was added to the culture medium only at the time of cell seeding. LY333531 (cayman) at a final concentration of 1 μM and IWR-1-endo at a final concentration of 20 μM were added to the medium only on days 2 and 3 of culture. The cells were treated for 3 minutes with TrypLE™ Select Enzyme (Life Technologies Japan Ltd.) supplemented with 10 μM of Y-27632 (FUJIFILM Wako Pure Chemical Corporation), and the cells were detached from the culture surface by tapping, and being dispersed as single cells by pipetting. The cells were suspended and collected in StemFit (registered trademark) AK03N (AJINOMOTO CO., INC.) containing Y-27632 at a final concentration of 10 μM.

### Production Example 3: Suspension Culture of Human iPS Cell Line Ff-I14s04

The cells cultured and collected in Production Example 2 were seeded for suspension culture. BioBlu 1c Single-Use Vessel (Eppendorf SE) was used as a culture vessel. Bioflo (Eppendorf SE) was used as a reactor system for controlling culture. A pH sensor and a medium perfusion pump provided with Bioflo were calibrated according to the method specified by the manufacturer. The cells were seeded such that the culture solution amount was 320 mL and the cell density at the start of culture was 1.25×10⁵ cells/mL, and then the culture was started. StemFit (registered trademark) AK03N (AJINOMOTO CO., INC.) supplemented with Y-27632 at a final concentration of 10 μM and IWR-1-endo at a final concentration of 20 μM was used as a medium for seeding. During the culture, the culture temperature was maintained at 37° C., and the amount of gas supplied was maintained at 0.2 L/min to perform top surface aeration of the culture solution. The carbon dioxide gas concentration in the gas supplied was 5% at the start of culture, and then adjusted upward or downward to decrease as shown in FIG. 1 such that the pH of the culture solution was maintained at or around 7.15 (a decrease in the pH was suppressed). The gas supplied was prepared by mixing air with an arbitrary volume of carbon dioxide gas. The stirring rate was set to 75 rpm until hour 48 of culture and 68 rpm afterward. The start of culture was set at hour 0 of culture, and medium perfusion was started at hour 24 of culture. The amount of perfusion per unit time was managed at 1-hour intervals to control the culture environment, as shown in Table 1. The amount of the medium perfused per unit time at the start of perfusion of was F₀=13.3 mL that is a value obtained by dividing the culture volume of 320 mL by 24 hours. The time point at which the amount of the medium perfused per unit time started to be changed was set at hour 35 of culture. Using the formula of Formula 3, the subsequent amount of the medium perfused per unit time was set on the basis of the following: a seeding density of 1.25×10⁵ cells/mL as C₀; 150% as a proportion of the number of cells forming cell aggregates at hour 24 of culture with respect to the assumed number of cells seeded; and the number of the cells at hour 35 of culture, in which the number is calculated from an assumed specific growth rate of 0.90 day⁻¹. C was the number of cells calculated from the transition of the assumed cell density, in which the transition was predicted in the same manner. A constant M for correcting the influence of cell lines and the like was set to 1. In addition, the amount of the medium perfused per unit time was set with K calculated by setting, from values for a common pluripotent stem cell, the amount L₀ of lactic acid produced by metabolism per cell per unit time, and the amount L of lactic acid produced by metabolism per cell per unit time in each culture time. The composition of the medium used for perfusion was switched between hours 24 to 48 of culture and hour 48 of culture and later such that the concentration of Y-27632 was different, 5 μM for hours 24 to 48 of culture and 2.5 μM for hour 48 of culture or later. StemFit (registered trademark) AK03N (AJINOMOTO CO., INC.) supplemented with IWR-1-endo at a final concentration of 20 μM and LY333531 at a final concentration of 1 μM was used for both media. To remove the medium and not the cell aggregate by suctioning from the culture solution, the medium was removed by passing through a sintered wire mesh filter having an opening of 30 μm.

TABLE 1

| Elapsed Culture Time | Flow Rate |
|---|---|
| (d · h:m:s) | (mL/h) |
| 1.00:00:00 | 13.33 |
| 1.01:00:00 | 13.33 |
| 1.02:00:00 | 13.33 |
| 1.03:00:00 | 13.33 |
| 1.04:00:00 | 13.33 |
| 1.05:00:00 | 13.33 |
| 1.06:00:00 | 13.33 |
| 1.07:00:00 | 13.33 |
| 1.08:00:00 | 13.33 |
| 1.09:00:00 | 13.33 |
| 1.10:00:00 | 13.33 |
| 1.11:00:00 | 13.84 |
| 1.12:00:00 | 14.37 |
| 1.13:00:00 | 14.92 |
| 1.14:00:00 | 15.49 |
| 1.15:00:00 | 16.08 |
| 1.16:00:00 | 16.70 |
| 1.17:00:00 | 17.34 |
| 1.18:00:00 | 18.00 |
| 1.19:00:00 | 18.69 |
| 1.20:00:00 | 19.40 |
| 1.21:00:00 | 20.14 |
| 1.22:00:00 | 20.91 |
| 1.23:00:00 | 21.71 |
| 2.00:00:00 | 22.54 |
| 2.01:00:00 | 23.40 |
| 2.02:00:00 | 24.29 |
| 2.03:00:00 | 25.22 |
| 2.04:00:00 | 26.19 |
| 2.05:00:00 | 27.19 |
| 2.06:00:00 | 28.23 |
| 2.07:00:00 | 29.31 |
| 2.08:00:00 | 30.43 |
| 2.09:00:00 | 31.59 |
| 2.10:00:00 | 32.79 |
| 2.11:00:00 | 34.05 |
| 2.12:00:00 | 35.35 |
| 2.13:00:00 | 36.70 |
| 2.14:00:00 | 38.10 |
| 2.15:00:00 | 39.56 |
| 2.16:00:00 | 41.07 |
| 2.17:00:00 | 42.64 |
| 2.18:00:00 | 44.27 |
| 2.19:00:00 | 45.96 |
| 2.20:00:00 | 47.72 |
| 2.21:00:00 | 49.54 |
| 2.22:00:00 | 51.43 |
| 2.23:00:00 | 53.40 |
| 3.00:00:00 | 55.44 |
| 3.01:00:00 | 57.56 |
| 3.02:00:00 | 59.76 |
| 3.03:00:00 | 62.04 |

At hour 75 of culture, the whole amount of the culture solution in the suspension culture was collected. The cell aggregates and the medium were separated by centrifugation. Then, the cell aggregates were treated with TrypLE™ Select Enzyme (Life Technologies Japan Ltd.) under rotation for 5 minutes, and unicellularized by pipetting. Then, the cells were suspended in the medium, and collected.

### Production Example 4: Suspension Culture of Human iPS Cell Line Ff-I14s04

The cells subjected to suspension culture and collected in Production Example 3 were further subjected to suspension culture. A culture was performed in the same manner as in Production Example 3 except that the seeding density was changed to 1.50×10⁵ cells/mL, that LY333531 was added at a final concentration of 1 μM to the culture medium for seeding, and that the cells were collected at hour 72 of culture.

### Production Example 5: Suspension Culture of Human iPS Cell Line Ff-I14s04

The cells subjected to suspension culture and collected in Production Example 4 were further subjected to suspension culture. A culture was performed in the same manner as in Production Example 4.

### Example 1: Production of Stock of Human iPS Cell Line Ff-I14s04

In such a manner that the cell density was 1.0×10⁶ cells/mL, the cells subjected to suspension culture and collected in Production Example 3 were suspended in an amount of 330 mL in STEM-CELLBANKER (Zenogen Pharma Co., Ltd.) preliminarily cooled to approximately 4° C. Then, the STEM-CELLBANKER (hereinafter referred to as a stock solution) having the cells suspended therein was kept cooled on a cooling core (Corning, Inc.). Into each of 300 of NUNC Cryo Tube vials (Thermo Fisher Scientific Inc.) kept cooled on a cooling core in the same manner, 1 mL of the stock solution was aliquoted using an 8-channel electric PIPETMAN. Then, the solution was frozen using a program freezer at a cooling rate of 1° C./min to produce a cell stock.

### Example 2: Production of Stock of Human iPS Cell Line Ff-I14s04

A cell stock was produced in the same manner as in Example 1 using the cells subjected to suspension culture and collected in Production Example 4.

### Example 3: Production of Stock of Human iPS Cell Line Ff-I14s04

A cell stock was produced in the same manner as in Example 2 using the cells subjected to suspension culture and collected in Production Example 5.

### Evaluation Example 1: Measurement of Number of Cells Subjected to Suspension Culture

Using NC-200 (M&S TechnoSystems, Inc.), the number of viable cells and survival rate (a proportion of the number of viable cells with respect to the whole number of cells collected) of the cells collected in each of Production Examples 3, 4, and 5 were measured. Table 2 shows the results.

TABLE 2

| | Viable Cell Count (cells) | Survival Rate (%) |
|---|---|---|
| Production Example 3 | 4.47 × 10⁸ | 98 |
| Production Example 4 | 4.27 × 10⁸ | 98 |
| Production Example 5 | 4.39 × 10⁸ | 99 |

As shown in Table 2, a large number of cells were grown stably in the suspension culture by the peculiar suspension culture method in each period. Cells in approximately 50 of 10 cm² dishes in the adherent culture were successfully obtained from one reactor. That is, it has been indicated that using the method according to the present invention can mass-culture cells simply. In this regard, a suspension culture method according to the present invention can further prolong the culture time, and it can be said that the method has a very high superiority in simplicity over an adherent culture. In addition, cells were successfully grown efficiently at a very fast rate approximately 10 times as high in three days of culture. That is, it has been indicated that the method according to the present invention can efficiently and simply produce a large number of cell stocks from a very small number of raw material cells. Further, it has been revealed that cells having a very high survival rate and high quality were cultured successfully.

### Evaluation Example 2: Confirmation of Positive Rate of Cells Subjected to Suspension Culture, for Undifferentiation Marker

The cells collected in Production Examples 3, 4, and 5 were subjected to fixation, permeabilization, and blocking using eBioscience Foxp3 Transcription Factor Staining Buffer Set (Thermo Fisher Scientific Inc.). Then, the cell sample was divided, and the resulting samples were each resuspended to 50 μL using a buffer provided with the eBioscience Foxp3 Transcription Factor Staining Buffer Set (Thermo Fisher Scientific Inc.). Fluorescently-labeled anti-OCT4, anti-SOX2, and anti-NANOG antibodies were added to and mixed with one of the samples, and FMO controls were prepared by mixing antibodies in which one antibody was removed from the three fluorescently-labeled antibodies with each of three samples, respectively. Staining was performed at 4° C. for 1 hour in a light-proof state. Table 3 lists the antibodies used and their amounts added.

TABLE 3

| | Manufacturer, Model Number | Amount Added | |
|---|---|---|---|
| Fluorescence-labeled | Becton, Dickinson and | 10 | μL |
| Anti-OCT4 Antibody | Company, 560186 | | |
| Fluorescence-labeled | BioLegend, Inc., | 1 | μL |
| Anti-SOX2 Antibody | 656110 | | |
| Fluorescence-labeled | Becton, Dickinson and | 2.5 | μL |
| Anti-NANOG Antibody | Company, 561300 | | |

After washing once with 3% FBS (fetal bovine serum)/PBS, the cells passed through a cell strainer were analyzed using Guava easyCyte 8HT (Luminex Corporation). For the FMO control samples, all regions where the cell population with the strong fluorescence intensity was 0.5% or less were selected in the cell populations extracted by the FSC/SSC dot plot. For samples treated with the anti-OCT4, anti-SOX2, and anti-NANOG antibodies, the proportion of cells contained within the regions was calculated in the cell populations extracted from the FSC/SSC dot plot. This was defined as the proportion of cells positive for OCT4, SOX2, and NANOG. Table 4 shows the results.

TABLE 4

| | Proportion of | Proportion of | Proportion of |
|---|---|---|---|
| | Cells Positive | Cells Positive | Cells Positive |
| | for OCT4 (%) | for SOX2 (%) | for NANOG (%) |
| Production | 99 | 100 | 99 |
| Example 3 | | | |
| Production | 99 | 100 | 98 |
| Example 4 | | | |
| Production | 99 | 100 | 97 |
| Example 5 | | | |

As shown in Table 4, the undifferentiation markers OCT4, SOX2, and NANOG have indicated that all the cells obtained in Production Example 3, Production Example 4, and Production Example 5 had a very high proportion of cells positive for the markers, that a cell population highly maintaining the undifferentiated property can be cultured in suspension culture in which the undifferentiated property is generally difficult to maintain, and that the method according to the present invention can produce a high-quality cell stock.

### Evaluation Example 3: Confirmation of Suppression of Differentiation of Cells Subjected to Suspension Culture

The cells collected in Production Examples 3, 4, and 5 were dissolved using TRIzol™ Reagent (Thermo Fisher Scientific Inc.). Total RNA was isolated and purified using PureLink (registered trademark) RNA Mini Kit (Thermo Fisher Scientific Inc.) from the solution of the cells dissolved with TRIzol™ Reagent. The concentration of purified RNA was measured using BioSpec-nano (SHIMADZU CORPORATION), thereby fractionating 500 ng of purified RNA. A solution in an amount of 10 μL was prepared with the adding 2 μL of ReverTra Ace (registered trademark) qPCR RT Master mix (TOYOBO CO., LTD.) and Rnase Free dH₂O to the fractionated RNA. cDNA synthesis was performed using SimpliAmp Thermal Cycler (Thermo Fisher Scientific Inc.). Reaction conditions for cDNA synthesis was as follows: a reaction at 37° C. was performed for 15 minutes, and then a reaction at 50° C. for 5 minutes and a reaction at 98° C. for 5 minutes were performed sequentially, followed by cooling to 4° C. The synthesized cDNA solution was diluted 100-fold with 10 mM Tris-HCl (pH 8.0; NACALAI TESQUE, INC.) and added at 5 μL/well to a 384-well PCR plate (Thermo Fisher Scientific Inc.). KOD SYBR (registered trademark) qPCR Mix (TOYOBO CO., LTD.), a forward primer prepared to be 50 μM, a reverse primer prepared to be 50 μM, and DEPC-treated water (NACALAI TESQUE, INC.) were mixed at a ratio of 100:1:1:48. This mixed solution was added at 15 μL/well to the 384-well PCR plate and mixed. ACTB, OCT4, SOX2, NANOG, and HK2 were used as primers. The 384-well PCR plate was centrifuged to remove air bubbles in the wells, and quantitative real-time PCR analysis was performed using QuantStudio 7 Flex Real-Time PCR System (Thermo Fisher Scientific Inc.). Table 5 lists the reaction conditions.

TABLE 5

| | | | Number of |
|---|---|---|---|
| Step | Temperature | Time | Cycles |
| 1 Initial Denaturation | 98° C. | 1 minute | — |
| 2 Denaturation | 98° C. | 15 seconds | 5 cycles |
| 3 Annealing, Elongation | 68° C. | 30 seconds | |
| 4 Denaturation | 98° C. | 15 seconds | 40 cycles |
| 5 Annealing | 60° C. | 10 seconds | |
| 6 Elongation | 68° C. | 30 seconds | |
| 7 Melting Curve | 95° C. | 15 seconds | — |
| | 60° C. | 1 minute | |
| | 98° C. | 15 seconds | |

The base sequences of the primers used for quantitative real-time PCR analysis are shown below.

ACTB (Forward):
(SEQ ID NO: 1)
5′-CCTCATGAAGATCCTCACCGA-3′
ACTB (Reverse):
(SEQ ID NO: 2)
5′-TTGCCAATGGTGATGACCTGG-3′
PAX6 (Forward):
(SEQ ID NO: 3)
5′-AGGAATGGACTTGAAACAAGG-3′
PAX6 (Reverse):
(SEQ ID NO: 4)
5′-GCAAAGCTTGTTGATCATGG-3′
BRACHYURY (Forward):
(SEQ ID NO: 5)
5′-TCACAAAGAGATGATGGAGGAAC-3′
BRACHYURY (Reverse):
(SEQ ID NO: 6)
5′-ACATGCAGGTGAGTTGTCAG-3′
SOX17 (Forward):
(SEQ ID NO: 7)
5′-ATCTGCACTTCGTGTGCAAG-3′
SOX17 (Reverse):
(SEQ ID NO: 8)
5′-GAGTCTGAGGATTTCCTTAGCTC-3′

Table 6 shows the measurement results.

TABLE 6

| | PAX6 | BRACHYURY | SOX17 |
|---|---|---|---|
| | (Ectoderm) | (Mesoderm) | (Endoderm) |
| Production Example 3 | not detected | not detected | not detected |
| Production Example 4 | not detected | not detected | not detected |
| Production Example 5 | not detected | not detected | not detected |

As shown in Table 6, no differentiation marker gene was detected with the iPS cell cultured in each suspension culture according to the present invention (the detection limit: 1.0×10⁻⁵ or less). Not only from the viewpoint of such an undifferentiation marker as shown in Evaluation Example 2 but also from the viewpoint of a differentiation marker, it has been indicated that a cell stock maintaining the undifferentiated property can be produced. That is, it has been confirmed that the undifferentiated property of the iPS cell produced by the method according to the present invention has high quality on a more stringent criterion.

### Evaluation Example 4: Confirmation of Three-Germ-Layer Differentiation Potency of Cell Subjected to Suspension Culture

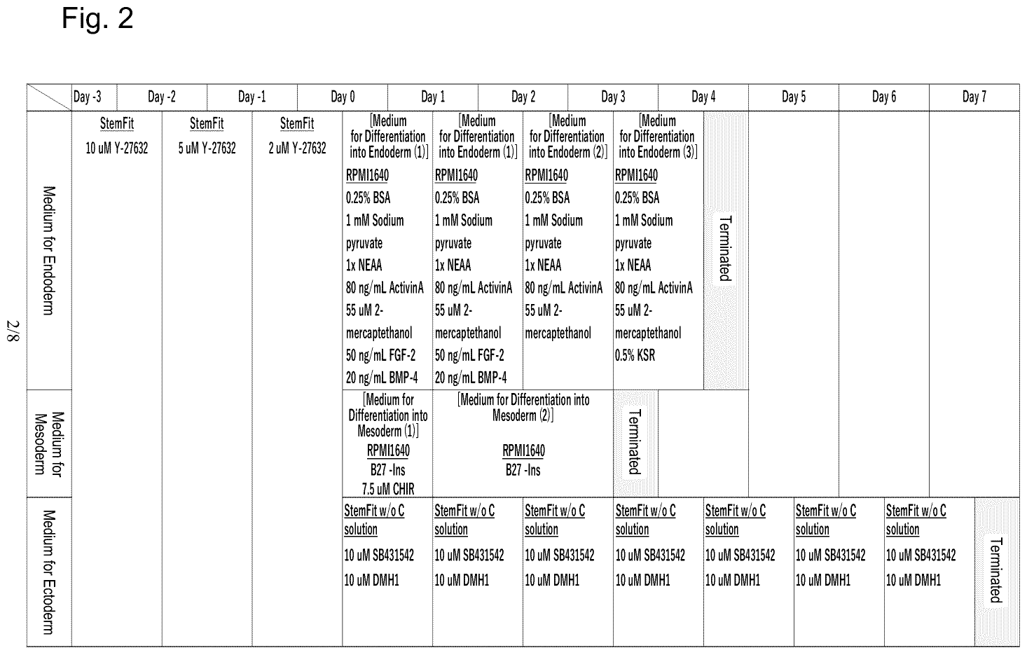

A cell stock produced in each of Examples 1, 2, and 3 was thawed, and then seeded in a 6-well plate at 2.0×10⁵ cells/mL in a culture solution in an amount of 4 mL/well, cultured under rotation using a rotary shaker at 90 rpm, and induced to differentiate into three germ layers. The media used were each a medium shown in FIG. 2. The cells were collected at the end of culture respectively shown in FIG. 2. The cell stock produced in each of Examples 1, 2, and 3 was dissolved in a TRIzol™ Reagent (Thermo Fisher Scientific Inc.) to prepare a DNA for quantitative real-time PCR in the same manner as the method described in Evaluation Example 3, on which DNA, a quantitative real-time PCR analysis was performed.

The base sequences of the primers used for quantitative real-time PCR analysis are shown below.

ACTB (Forward):
(SEQ ID NO: 1)
5′-CCTCATGAAGATCCTCACCGA-3′
ACTB (Reverse):
(SEQ ID NO: 2)
5′-TTGCCAATGGTGATGACCTGG-3′
PAX6 (Forward):
(SEQ ID NO: 3)
5′-AGGAATGGACTTGAAACAAGG-3′
PAX6 (Reverse):
(SEQ ID NO: 4)
5′-GCAAAGCTTGTTGATCATGG-3′
PDGFRα (Forward):
(SEQ ID NO: 9)
5′-GCTGAGCCTAATCCTCTGCC-3′
PDGFRα (Reverse):
(SEQ ID NO: 10)
5′-ACTGCTCACTTCCAAGACCG-3′
SOX17 (Forward):
(SEQ ID NO: 7)
5′-ATCTGCACTTCGTGTGCAAG-3′
SOX17 (Reverse):
(SEQ ID NO: 8)
5′-GAGTCTGAGGATTTCCTTAGCTC-3′

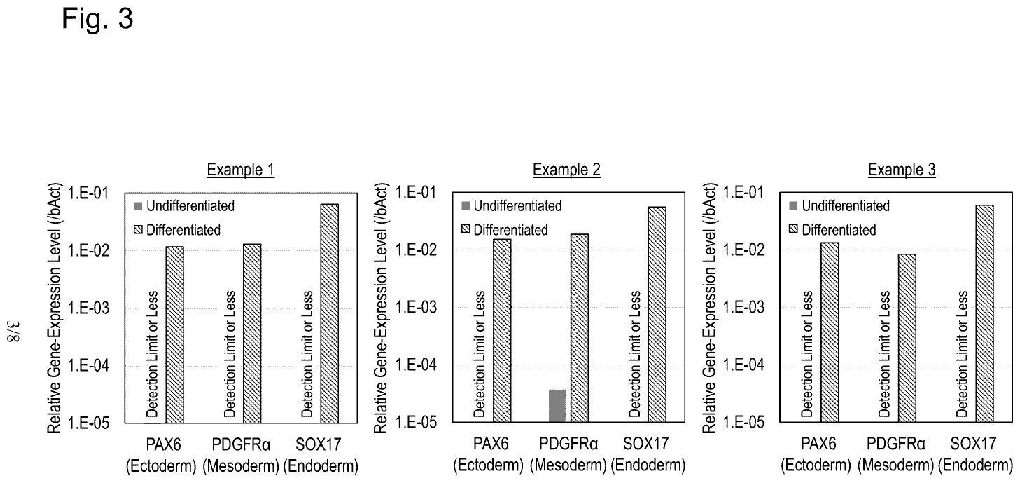

FIG. 3 shows the results of gene expression measurement. As shown in FIG. 3, all the cell stocks produced by the method according to the present invention in Examples 1, 2, and 3 respectively expressed three germ layer markers noticeably after being induced to differentiate, successfully confirming that the iPS cell stock produced by the method according to the present invention retains the three-germ-layer differentiation potency.

### Evaluation Example 5: Confirmation of Specific Growth Rate Immediately Before Production of Suspension Culture Stock

The cell aggregates at hour 48 of culture in Production Examples 3, 4, and 5 were collected and treated with Accutase (Innovative Cell Technologies, Inc.) for 10 minutes, and unicellularized by pipetting. The cells were suspended in StemFit (registered trademark) AK02N (AJINOMOTO CO., INC.) containing Y-27632 at a final concentration of 10 μM, and the viable cell number was measured using NC-200. From the results and the number of cells at the end of the culture in each of Production Examples 3, 4, and 5 as measured in Evaluation Example 1, a specific growth rate of the cells in suspension culture immediately before production of a stock was calculated. Table 7 shows the results.

TABLE 7

| | Specific Growth Rate Immediately |
|---|---|
| | before Production of Stock (day⁻¹) |
| Production Example 3 | 0.94 |
| Production Example 4 | 0.87 |
| Production Example 5 | 0.91 |

As shown in Table 7, it is understood that the cells in the suspension culture in each Example had a very fast specific growth rate immediately before being produced into a stock. Usually, the specific growth rate is slow immediately before terminating culture in a passage period and sufficiently grown cells being collected, regardless of whether the culture is adherent culture or suspension culture. However, in the method according to the present invention, changes in the culture environment are grasped and managed, and thus, the cells can be cultured and used for a cell stock with the specific growth rate not decreased until the end of culture, that is, with the cells maintained in the best state. This enables a high-quality cell stock that is also mass-producible, which is conventionally difficult.

### Comparative Example 1: Adherent Culture and Stock Production of Human iPS Cell Line Ff-I14s04

Human iPS cells of the Ff-I14s04 cell line (The Center for iPS Cell Research and Application, Kyoto University), in a frozen state, were thawed, then seeded at 6000 cells/cm² in a 25 cm² culture flask coated with iMatrix-511 MG (Matrixome, Inc.) at 0.5 g/cm², and subjected to adherent culture at 37° C. in a 5% CO₂ atmosphere. StemFit (registered trademark) AK03N (AJINOMOTO CO., INC.) was used as a culture medium. Given that the day on which the cells were seeded was day 0 of culture, the whole amount of the culture medium was changed on day 1 of culture, day 3 of culture, and day 5 of culture. The amount of the medium was 5 mL. Y-27632 (FUJIFILM Wako Pure Chemical Corporation) was added to the medium at a final concentration of 10 μM only at the time of cell seeding. On day 6 of culture, the cells were treated for a passage for 5 minutes with TrypLE™ Select Enzyme (Life Technologies Japan Ltd.), and the cells were detached from the culture surface using a cell scraper, and dispersed as single cells. The cells were suspended and collected in StemFit (registered trademark) AK03N (AJINOMOTO CO., INC.) containing Y-27632 at a final concentration of 10 μM. Then, the cells were suspended in STEM-CELLBANKER (Zenogen Pharma Co., Ltd.) in such a manner that the cell density was 1.0×10⁶ cells/mL. Then, 1 mL of STEM-CELLBANKER (hereinafter referred to as a stock solution) having cells suspended therein was aliquoted in each of the NUNC Cryo Tubes (Thermo Fisher Scientific Inc.), and frozen using a program freezer at a cooling rate of 1° C./min to produce a cell stock by a conventional adherent culture method.

### Evaluation Example 6

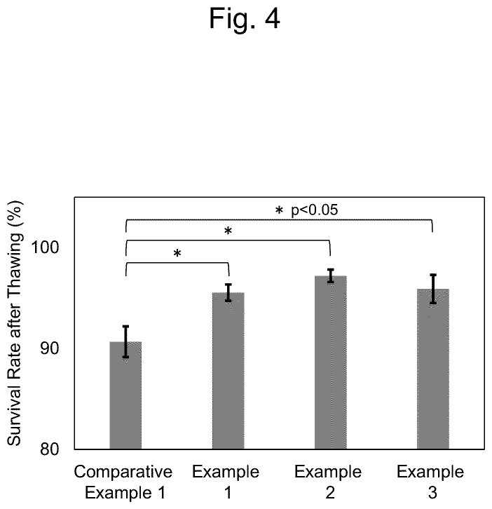

The cell stocks produced in Examples 1, 2, and 3 and the cell stock produced in Comparative Example 1 were each thawed, and then, the cell survival rate was measured using NC-200. Table 8 and FIG. 4 show the results.

TABLE 8

| | Survival Rate after |
|---|---|
| | Thawing of Stock (%) |
| Comparative Example 1 | 91 |
| Example 1 | 96 |
| Example 2 | 97 |
| Example 3 | 96 |

As shown in Table 8 and FIG. 4, it has been revealed that the stocks produced by the method according to the present invention in Examples 1, 2, and 3 had a higher survival rate after thawing than the stock produced by a conventional method in Comparative Example 1, and that the method according to the present invention can produce a high-quality cell stock.

### Evaluation Example 7: Adhesion Rate of Cell Stock to Adherent Culture

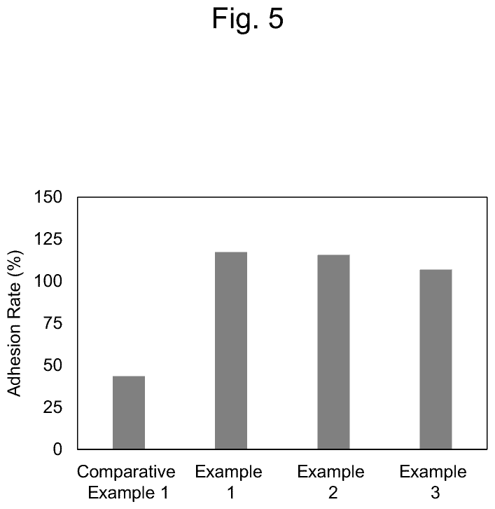

The cell stocks produced in Examples 1, 2, and 3 and the cell stocks produced in Comparative Example 1 were each thawed, then seeded at 6000 cells/cm² in a 25 cm² culture flask coated with iMatrix-511 (Matrixome, Inc.) at 0.5 μg/cm², and subjected to adherent culture at 37° C. in a 5% CO₂ atmosphere. As the medium, 5 mL of StemFit (registered trademark) AK02N (AJINOMOTO CO., INC.) supplemented with Y-27632 (FUJIFILM Wako Pure Chemical Corporation) at a final concentration of 10 μM was used. The day when the cells were seeded was regarded as day 0 of culture. The culture flask was observed on day 1 of culture, and the number of cells adhering was counted in the field of view, and converted into the number of cells adhering in the whole culture vessel. Then, the adhesion rate (a proportion of cells adhering at hour 24 of culture with respect to the number of cells seeded) was calculated. Table 9 and FIG. 5 show the results.

TABLE 9

| | Adhesion Rate (%) |
|---|---|
| Comparative Example 1 | 44 |
| Example 1 | 117 |
| Example 2 | 116 |
| Example 3 | 107 |

As shown in Table 9 and FIG. 5, it has been revealed that the cell stocks produced by the method according to the present invention in Examples 1, 2, and 3 had a noticeably high adhesion rate and high quality, compared with the cell stock produced by a conventional method in Comparative Example 1. In addition, the adhesion rate was surprisingly more than 100%, thus suggesting that the cells seeded already started growing immediately after seeding. Producing a cell stock by the method according to the present invention can achieve both reduction in the number of days of culture and higher efficiency when differentiated cells are produced using a stock.

### Production Example 6: Adherent Culture of Human iPS Cell Line Ff-I14s04

Human iPS cells of the Ff-I14s04 cell line (The Center for iPS Cell Research and Application, Kyoto University), in a frozen state, were thawed, then seeded at 1000 cells/cm² in a 150 cm² culture flask coated with iMatrix-511 MG (Matrixome, Inc.) at 0.5 μg/cm², and subjected to adherent culture at 37° C. in a 5% CO₂ atmosphere. StemFit (registered trademark) AK03N (AJINOMOTO CO., INC.) was used as a medium. Given that the day on which the cells were seeded was day 0 of culture, the whole amount of the medium was changed on day 1 of culture, day 4 of culture, day 6 of culture, day 8 of culture, day 9 of culture, and day 10 of culture. The amount of the medium was 30 mL. Y-27632 (FUJIFILM Wako Pure Chemical Corporation) was added to the medium at a final concentration of 10 μM only at the time of cell seeding. On day 11 of culture, the cells were treated for a passage for 5 minutes with TrypLE™ Select Enzyme (Life Technologies Japan Ltd.), and the cells were detached from the culture surface using a cell scraper, and dispersed as single cells. The cells were suspended and collected in StemFit (registered trademark) AK03N (AJINOMOTO CO., INC.) containing Y-27632 at a final concentration of 10 μM.

### Evaluation Example 8: Cell Yield in Adherent Culture Using Raw Material Cell

The number of the viable cells of the cells collected cultured in Production Example 2 and Production Example 6 was measured using NC-200. Table 10 shows the results.

TABLE 10

| | Number of Viable Cells |
|---|---|
| | Collected (cells) |
| Production Example 2 | 4.12 × 10⁷ |
| Production Example 6 | 2.12 × 10⁷ |

As shown in Table 10, the number of cells obtained was larger in Production Example 2 than in Production Example 6, in which, in Production Example 2, raw material cells were seeded and cultured at a high seeding density in a small-scale adherent culture, passaged to a middle-scale culture, cultured for a total of two passage periods, that is, a total of 10 days, and collected, and in Production Example 6, cells were seeded and cultured at a lower seeding density in a middle-scale adherent culture, and cultured for a total of one passage period, that is, a total of 11 days. In this regard, in both of Production Example 2 and Production Example 6, the coverage rate of the cells to the culture surface was approximately 60% when the cells were collected. That is, it has been indicated that, when a small number of raw material cells are used, seeding the cells more densely in a small vessel, followed by culturing stepwise, tends to enable more efficient culture than seeding the cells less densely in a large vessel, followed by culturing for a long period of time, and affords a sufficiently large number of cells usable in the subsequent suspension culture.

### Evaluation Example 9: Undifferentiated Property of Cell in Adherent Culture Using Raw Material Cell

The cells collected in the culture in Production Example 2 and Production Example 6 were analyzed by flow cytometry in the same manner as the method in Evaluation Example 2. Table 11 shows the results.

TABLE 11

| | Proportion of | Proportion of |
|---|---|---|
| | Cells Positive | Cells Positive |
| | for OCT4 (%) | for NANOG (%) |
| Production Example 2 | 98 | 99 |
| Production Example 6 | 87 | 82 |

As shown in Table 11, the cells in Production Example 6 exhibited a slight decrease in the positive rate for the undifferentiation marker, thus suggesting the possibility that the quality of the cells would decrease before the suspension culture step. This is considered to be because seeding raw material cells at a lower seeding density rendered the state of the cells unstable, and because increasing the number of days of culture in one passage period to sufficiently grow cells caused the cells in each cell colony to be denser, and damaged. These considerations indicate the usefulness of a more preferable method according to the present invention, which involves seeding cells at a high seeding density and performing adherent culture.

### Production Example 7: Suspension Culture of Human iPS Cell Line Ff-I14s04

Human iPS cells of the Ff-I14s04 cell line (Kyoto University, The Center for iPS Cell Research and Application) were subjected to adherent culture in the same manner as in Production Example 1 except that a 150 cm² culture flask was used, that the cells were seeded at 1000 cells/cm², that iMatix-511 was mixed at 0.25 μg/cm² with the medium when the cells were seeded, and that the cells were collected on day 8 of culture. The cells were prepared so as to contain 1×10⁵ cells per 1 mL, using StemFit (registered trademark) AK02N (AJINOMOTO CO., INC.) containing Y-27632 at a final concentration of 10 μM and IWR-1-endo at a final concentration of 20 μM. In each BioBlu 1c Single-Use Vessel (Eppendorf SE), 320 mL of the cell suspension was seeded. The cells seeded were stirred in a reactor at 75 rpm, and subjected to suspension culture at a temperature of 37° C. in a 5% CO₂ gas environment. StemFit (registered trademark) AK02N (AJINOMOTO CO., INC.) was used as a medium. Given that the day on which the cells were seeded was day 0 of culture, the whole amount of the medium was changed on day 1 of culture and day 2 of culture. For medium change on day 1 of culture, StemFit (registered trademark) AK02N (AJINOMOTO CO., INC.) containing Y-27632 at a final concentration of 7 μM, IWR-1-endo at a final concentration of 20 μM, and LY333531 at a final concentration of 1 μM was used. For medium change of day 2 of culture, StemFit (registered trademark) AK02N (AJINOMOTO CO., INC.) containing Y-27632 at a final concentration of 3 μM, IWR-1-endo at a final concentration of 20 μM, and LY333531 at a final concentration of 1 μM was used. On day 3 of culture, the cells were treated for a passage for 5 minutes with TrypLE™ Select Enzyme (Life Technologies Japan Ltd.), and the cell aggregates were dispersed as single cells by pipetting. The cells were suspended and collected in StemFit (registered trademark) AK02N (AJINOMOTO CO., INC.) containing Y-27632 at a final concentration of 10 μM.

### Example 4: Low-Temperature Preparation of Stock Solution

In such a manner that the cell density was 1.0×10⁶ cells/mL, the cells subjected to suspension culture and collected in Production Example 7 were suspended in STEM-CELLBANKER (Zenogen Pharma Co., Ltd.) preliminarily kept in cold storage at a temperature of approximately 4° C. Then, STEM-CELLBANKER (a stock solution) having cells suspended therein was kept cooled on a cooling core (Corning, Inc.). Into a NUNC Cryo Tube vial (Thermo Fisher Scientific Inc.) kept cooled on a cooling core in the same manner, 1 mL of the stock solution was aliquoted. Then, in order to produce a cell stock, the cells, kept cooled on the cooling core, were slowly frozen at a cooling rate of 1° C./m using Mr. Frosty (Thermo Fisher Scientific Inc.) after 10, 30, 60, and 120 minutes from the time point when the cells were suspended in the STEM-CELLBANKER.

### Example 5: Room-Temperature Preparation of Stock Solution

A cell stock was produced in the same manner as in Example 4 except that, for the cells subjected to suspension culture, and collected in Production Example 7, preparation of a stock solution and standby until freezing were performed at room temperature (22° C.).

### Evaluation Example 10: Confirmation of Cell Survival Rate Before and After Production of Cell Stock

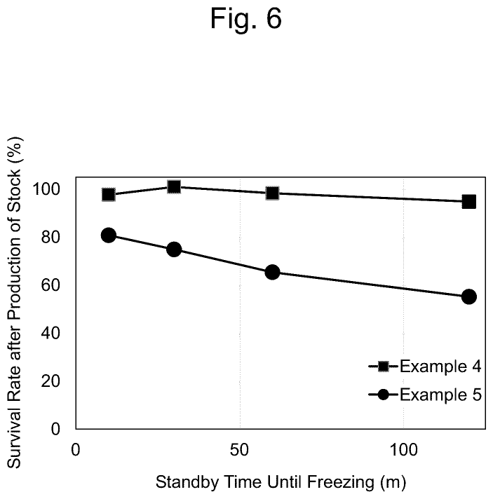

The cell stocks produced in Example 4 and Example 5 were each thawed, and then, the viable cell number was measured using NC-200. From the measurement results, a proportion of the viable cell number after stock production and thawing with respect to the viable cell number after filling was calculated. FIG. 6 shows the results.

As shown in FIG. 6, the results obtained were that, with the cell stock prepared at room temperature in accordance with a conventional method, the number of viable cells contained in a cell stock produced tended to decrease as the standby time until freezing was longer, which is the filling work time required when a large number of cell stocks were produced, but on the other hand, the cell stock prepared in a cold-storage state by a more preferable method according to the present invention did not cause the number of viable cells to decrease with elapse of time. It is considered to be because the cold storage can suppress a decrease in the apoptosis or the like of the cells. That is, a more preferable method according to the present invention makes it possible that, using a high-quality pluripotent stem cell amplified in a culture step, a cell stock is produced without impairing the quality of the stem cell.

### Evaluation Example 11: Aggregate-Forming Rate of Cell Stock in Suspension Culture

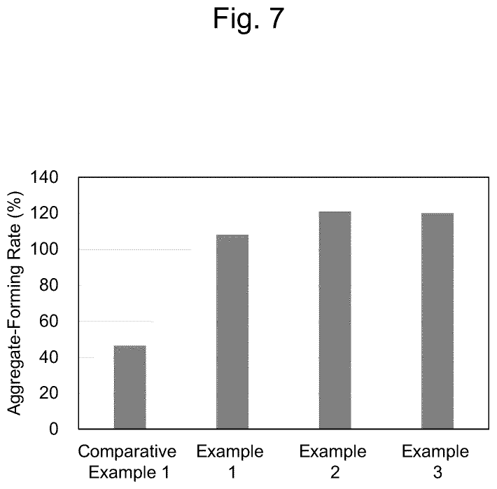

The cell stocks produced in Examples 1, 2, and 3 and the cell stock produced in Comparative Example 1 were each thawed, then seeded at 10,000 cells/mL in a 30 mL reactor (ABLE Corporation), and subjected to suspension culture under stirring at 100 rpm at 37° C. in a 5% CO₂ atmosphere. As the medium, StemFit (registered trademark) AK02N (AJINOMOTO CO., INC.) supplemented with Y-27632 (FUJIFILM Wako Pure Chemical Corporation) at a final concentration of 10 μM was used. The day of cell seeding was set as day 0 of culture. Cell aggregates formed on day 1 of culture were collected, treated with Accutase (Innovative Cell Technologies, Inc.) for 10 minutes, and unicellularized by pipetting. The cells were suspended in StemFit (registered trademark) AK02N (AJINOMOTO CO., INC.) containing Y-27632 at a final concentration of 10 μM, and the viable cell number was measured using NC-200. From the viable cell number measured, the aggregate-forming rate (a proportion, to the number of cells seeded, of the number of cells in the form of cell aggregates at hour 24 of culture) was calculated. Table 12 and FIG. 7 show the results.

TABLE 12

| | Aggregate-forming Rate (%) |
|---|---|
| Comparative Example 1 | 46 |
| Example 1 | 123 |
| Example 2 | 111 |
| Example 3 | 127 |

As shown in Table 12 and FIG. 7, it has been revealed that the cell stocks produced by the method according to the present invention in Examples 1, 2, and 3 had a noticeably high aggregate-forming rate and high quality, compared with the cell stock produced by a conventional method in Comparative Example 1. In addition, the aggregate-forming rate was surprisingly more than 100%, thus suggesting that the cells seeded already started growing immediately after seeding. Producing a cell stock by the method according to the present invention can achieve both reduction in the number of days of culture and higher efficiency when differentiated cells are produced using a stock.

### Evaluation Example 12: Cell Cycle Analysis of Cell Stock

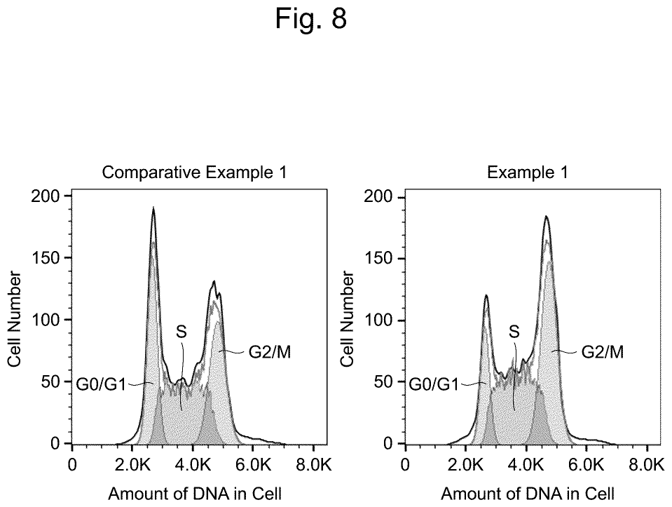

The cell stock produced in Example 1 and the cell stock produced in Comparative Example 1 were each thawed, and then stained with Cell Meter Fluorimetric Fixed Cell Cycle Assay Kit (AAT Bioquest, Inc.). Then, the cell stocks were analyzed with Guava easyCyte 8HT (Luminex Corporation). Cell populations extracted by the FSC/SSC dot plot using Flowjo (Becton, Dickinson and Company) were subjected to cell cycle analysis by the Watson (Pragmatic) method. Table 13 and FIG. 8 show the results. As the amounts of DNA at the boundaries of the cell populations between the G2/M phase and the S phase and between the G0/G phase and the S phase, the amounts of DNA corresponding to the peaks of the overlapped regions of each shaded part in FIG. 8 were used.

TABLE 13

| | G0/G1 Phase | S Phase | G2/M Phase |
|---|---|---|---|
| | (%) | (%) | (%) |
| Comparative Example 1 | 26.8 | 40.4 | 28.8 |
| Example 1 | 15 | 41.6 | 39.8 |

As shown in Table 13, it has been revealed that, compared with the cell stock produced by a conventional method in Comparative Example 1, the cell stock produced by the method according to the present invention in Example 1 was characterized in that a proportion of the cells in the G2/M phase was high, and that the ratio of a proportion in the G2/M phase with respect to a proportion in the G0/G1 phase was high.

All publications, patents, and patent applications cited herein are incorporated herein by reference in their entirety.

### SEQUENCE LISTING

1. A method for producing a pluripotent stem cell stock, comprising:
(a) a step of thawing frozen cells as a raw material for a cell stock to be produced to obtain thawed raw material cells;
(b) a step of adherent culture of the thawed raw material cells;
(c) a step of suspension culture of the cells subjected to the adherent culture;
(d) a step of aliquoting the cells subjected to the suspension culture into a vessel for storing the stock; and
(e) a step of freezing the cells aliquoted into the vessel.

2. The method for producing according to claim 1, wherein
the number of the thawed raw material cells used in the step (b) is 1×10⁶ cells or less, and
the number of the cells upon termination of the culture in the step (c) is 1×10⁸ cells or more.

3. The method for producing according to claim 1, wherein the adhesion rate of the raw material cells is 70% or less.

4. The method for producing according to claim 1, wherein, in the adherent culture in the step (b), the thawed cells are seeded at a density of 3×10³ cells/cm² or more.

5. The method for producing according to claim 1, wherein the step (b) comprises a passage.

6. The method for producing according to claim 5, wherein the passage is a passage into a vessel having a larger area.

7. The method for producing according to claim 1, wherein the suspension culture in the step (c) is performed by a mode of perfusing a medium.

8. The method for producing according to claim 7, wherein the mode of perfusing a medium comprises increasing the amount of medium perfused in accordance with the growth of the cells.

9. The method for producing according to claim 7, comprising controlling, by the mode of perfusing a medium, the amount of medium perfused so as to maintain the pH of a culture solution between 6.5 and 9.0.

10. The method for producing according to claim 1, wherein the suspension culture in the step (c) comprises altering the concentration of carbon dioxide gas supplied in the range of from 10 to 0%, in accordance with the progress of the culture.

11. The method for producing according to claim 1, wherein the suspension culture in the step (c) is suspension stirring culture.

12. The method for producing according to claim 11, wherein the suspension stirring culture comprises decreasing a stirring rate in a culture period.

13. The method for producing according to claim 1, wherein the amount of the culture solution in the suspension culture in the step (c) is 100 mL or more.

14. The method for producing according to claim 1, wherein a specific growth rate of the cell upon termination of the step (c) is 0.70 day⁻¹ or more.

15. The method for producing according to claim 1, wherein the step (c) comprises a step of unicellularizing a cell aggregate.

16. The method for producing according to claim 15, wherein the unicellularization comprises an enzymatic treatment in the presence of a ROCK inhibitor.

17. The method for producing according to claim 1, wherein, in the step (d), at least one of the vessels and a cell suspension is maintained at 10° C. or less.

18. The method for producing according to claim 1, wherein 100 or more of the pluripotent stem cell stock are produced.

19. The method for producing according to claim 1, wherein the aliquoting in the step (d) is performed using a multi-channel pipette.

20. The method for producing according to claim 1, wherein the medium used for the culture in the step (c) comprises a ROCK inhibitor.

21. The method for producing according to claim 20, wherein the ROCK inhibitor is Y-27632.

22. The method for producing according to claim 1, wherein the medium used for the culture in the step (b) and the step (c) comprises at least one selected from the group consisting of L-ascorbic acid, insulin, transferrin, selenium, and sodium hydrogen carbonate.

23. The method for producing according to claim 1, wherein the medium used for the culture in the step (b) and the step (c) comprises FGF2 and/or TGF-β1.

24. The method for producing according to claim 1, wherein a proportion of the cells positive for OCT4 is 90% or more, and a proportion of the cells positive for TRA-1-60 is 90% or more, in the pluripotent stem cells constituting the stock.

25. A method for improving the quality of a pluripotent stem cell, comprising:
(f) a step of adherent culture of a freeze-preserved pluripotent stem cell after being thawed; and
(g) a step of suspension culture of the cell subjected to the adherent culture.

26. A pluripotent stem cell stock comprising a cell, wherein
the survival rate of the cells is 90% or more after thawing, and
when the cells are used for adherent culture after the thawing, the number of the cells adhering at hour 24 of culture is 0.8 times or more to the number of the cells seeded.

27. A pluripotent stem cell stock comprising a cell, wherein the survival rate of the cells is 90% or more after thawing, and wherein, when the cells are used for suspension culture after the thawing, the number of viable cells at hour 24 of culture is 0.8 times or more to the number of the cells seeded.

28. A pluripotent stem cell stock, wherein, in respect of the cell cycle of the cell comprised, a proportion of the cells in the G2/M phase is 1.5 times or more with respect to a proportion of the cells in the G0/G1 phase.
